SEQUENCE LISTING (12 sequences)

SEQ ID NO: 1 - protein, 254 aa, Homo sapiens
MEYASDASLDPEAPWPPAPRARACRVLPWALVAGLLLLLLLAAACAVFLACPWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 2 - protein, 309 aa, Mus musculus
MDQHTLDVEDTADARHPAGTSCPSDAALLRDTGLLADAALLSDTVRPTNAALPTDAAYPAVNVRDREAAWPPALNFCSRHPKLYGLVALVLLLLIAACVPIFTRTEPRPALTITTSPNLGTRENNADQVTPVSHIGCPNTTQQGSPVFAKLLAKNQASLCNTTLNWHSQDGAGSSYLSQGLRYEEDKKELVVDSPGLYYVFLELKLSPTFTNTGHKVQGWVSLVLQAKPQVDDFDNLALTVELFPCSMENKLVDRSWSQLLLLKAGHRLSVGLRAYLHGAQDAYRDWELSYPNTTSFGLFLVKPDNPWE

SEQ ID NO: 3 - RNA, 1619 nt, Homo sapiens
  source 1..1619: mol_type = genomic RNA
gtcatggaatacgcctctgacgcttcactggaccccgaagccccgtggcctcccgcgccccgcgctcgcgcctgccgcgtactgccttgggccctggtcgcggggctgctgctgctgctgctgctcgctgccgcctgcgccgtcttcctcgcctgcccctgggccgtgtccggggctcgcgcctcgcccggctccgcggccagcccgagactccgcgagggtcccgagctttcgcccgacgatcccgccggcctcttggacctgcggcagggcatgtttgcgcagctggtggcccaaaatgttctgctgatcgatgggcccctgagctggtacagtgacccaggcctggcaggcgtgtccctgacggggggcctgagctacaaagaggacacgaaggagctggtggtggccaaggctggagtctactatgtcttctttcaactagagctgcggcgcgtggtggccggcgagggctcaggctccgtttcacttgcgctgcacctgcagccactgcgctctgctgctggggccgccgccctggctttgaccgtggacctgccacccgcctcctccgaggctcggaactcggccttcggtttccagggccgcttgctgcacctgagtgccggccagcgcctgggcgtccatcttcacactgaggccagggcacgccatgcctggcagcttacccagggcgccacagtcttgggactcttccgggtgacccccgaaatcccagccggactcccttcaccgaggtcggaataacgcccagcctgggtgcagcccacctggacagagtccgaatcctactccatccttcatggagacccctggtgctgggtccctgctgctttctctacctcaaggggcttggcaggggtccctgctgctgacctccccttgaggaccctcctcacccactccttccccaagttggaccttgatatttattctgagcctgagctcagataatatattatatatattatatatatatatatatttctatttaaagaggatcctgagtttgtgaatggacttttttagaggagttgttttggggggggggtcttcgacattgccgaggctggtcttgaactcctggacttagacgatcctcctgcctcagcctcccaagcaactgggattcatcctttctattaattcattgtacttatttgcctatttgtgtgtattgagcatctgtaatgtgccagcattgtgcccaggctagggggctatagaaacatctagaaatagactgaaagaaaatctgagttatggtaatacgtgaggaatttaaagactcatccccagcctccacctcctgtgtgatacttgggggctagcttttttctttctttcttttttttgagatggtcttgttctgtcaaccaggctagaatgcagcggtgcaatcatgagtcaatgcagcctccagcctcgacctcccgaggctcaggtgatcctcccatctcagcctctcgagtagctgggaccacagttgtgtgccaccacacttggctaactttttaatttttttgcggagacggtattgctatgttgccaaggttgtttacatgccagtacaatttataataaacactcatttttcc

SEQ ID NO: 4 - RNA, 1239 nt, Mus musculus
  source 1..1239: mol_type = genomic RNA
agcctataaagcacgggcactggcgggagacgtgcactgaccgaccgtggtaatggaccagcacacacttgatgtggaggataccgcggatgccagacatccagcaggtacttcgtgcccctcggatgcggcgctcctcagagataccgggctcctcgcggacgctgcgctcctctcagatactgtgcgccccacaaatgccgcgctccccacggatgctgcctaccctgcggttaatgttcgggatcgcgaggccgcgtggccgcctgcactgaacttctgttcccgccacccaaagctctatggcctagtcgctttggttttgctgcttctgatcgccgcctgtgttcctatcttcacccgcaccgagcctcggccagcgctcacaatcaccacctcgcccaacctgggtacccgagagaataatgcagaccaggtcacccctgtttcccacattggctgccccaacactacacaacagggctctcctgtgttcgccaagctactggctaaaaaccaagcatcgttgtgcaatacaactctgaactggcacagccaagatggagctgggagctcatacctatctcaaggtctgaggtacgaagaagacaaaaaggagttggtggtagacagtcccgggctctactacgtatttttggaactgaagctcagtccaacattcacaaacacaggccacaaggtgcagggctgggtctctcttgttttgcaagcaaagcctcaggtagatgactttgacaacttggccctgacagtggaactgttcccttgctccatggagaacaagttagtggaccgttcctggagtcaactgttgctcctgaaggctggccaccgcctcagtgtgggtctgagggcttatctgcatggagcccaggatgcatacagagactgggagctgtcttatcccaacaccaccagctttggactctttcttgtgaaacccgacaacccatgggaatgagaactatccttcttgtgactcctagttgctaagtcctcaagctgctatgttttatggggtctgagcaggggtcccttccatgactttctcttgtctttaactggacttggtatttattctgagcatagctcagacaagactttatataattcactagatagcattagtaaactgctgggcagctgctagataaaaaaaaatttctaaatcaaagtttatatttatattaatatataaaaataaatgtgtttgtaaat

SEQ ID NO: 5 - protein, 197 aa, Homo sapiens
RNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 6 - protein, 306 aa, Homo sapiens
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 7 - protein, 219 aa, Homo sapiens
MCPARSLLLVATLVLLDHLSLARNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 8 - protein, 328 aa, Homo sapiens
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 9 - protein, 432 aa, Homo sapiens
MERRRITSAARRSYVSSGEMMVGGLAPGRRLGPGTRLSLARMPPPLPTRVDFSLAGALNAGFKETRASERAEMMELNDRFASYIEKVRFLEQQNKALAAELNQLRAKEPTKLADVYQAELRELRLRLDQLTANSARLEVERDNLAQDLATVRQKLQDETNLRLEAENNLAAYRQEADEATLARLDLERKIESLEEEIRFLRKIHEEEVRELQEQLARQQVHVELDVAKPDLTAALKEIRTQYEAMASSNMHEAEEWYRSKFADLTDAAARNAELLRQAKHEANDYRRQLQSLTCDLESLRGTNESLERQMREQEERHVREAASYQEALARLEEEGQSLKDEMARHLQEYQDLLNVKLALDIEIATYRKLLEGEENRITIPVQTFSNLQIRETSLDTKSVSEGHLKRNIVVKTVEMRDGEVIKESKQEHKDVM

SEQ ID NO: 10 - protein, 431 aa, Homo sapiens
MERRRITSAARRSYVSSGEMMVGGLAPGRRLGPGTRLSLARMPPPLPTRVDFSLAGALNAGFKETRASERAEMMELNDRFASYIEKVRFLEQQNKALAAELNQLRAKEPTKLADVYQAELRELRLRLDQLTANSARLEVERDNLAQDLATVRQKLQDETNLRLEAENNLAAYRQEADEATLARLDLERKIESLEEEIRFLRKIHEEEVRELQEQLARQQVHVELDVAKPDLTAALKEIRTQYEAMASSNMHEAEEWYRSKFADLTDAAARNAELLRQAKHEANDYRRQLQSLTCDLESLRGTNESLERQMREQEERHVREAASYQEALARLEEEGQSLKDEMARHLQEYQDLLNVKLALDIEIATYRKLLEGEENRITIPVQTFSNLQIRGGKSTKDGENHKVTRYLKSLTIRVIPIQAHQIVNGTPPARG

SEQ ID NO: 11 - protein, 438 aa, Homo sapiens
MERRRITSAARRSYVSSGEMMVGGLAPGRRLGPGTRLSLARMPPPLPTRVDFSLAGALNAGFKETRASERAEMMELNDRFASYIEKVRFLEQQNKALAAELNQLRAKEPTKLADVYQAELRELRLRLDQLTANSARLEVERDNLAQDLATVRQKLQDETNLRLEAENNLAAYRQEADEATLARLDLERKIESLEEEIRFLRKIHEEEVRELQEQLARQQVHVELDVAKPDLTAALKEIRTQYEAMASSNMHEAEEWYRSKFADLTDAAARNAELLRQAKHEANDYRRQLQSLTCDLESLRGTNESLERQMREQEERHVREAASYQEALARLEEEGQSLKDEMARHLQEYQDLLNVKLALDIEIATYRKLLEGEENRITIPVQTFSNLQIRGQYSRASWEGHWSPAPSSRACRLLQTGTEDQGKGIQLSLGAFVTLQRS

SEQ ID NO: 12 - protein, 472 aa, Homo sapiens
MERRRITSAARRSYVSSGEMMVGGLAPGRRLGPGTRLSLARMPPPLPTRVDFSLAGALNAGFKETRASERAEMMELNDRFASYIEKVRFLEQQNKALAAELNQLRAKEPTKLADVYQAELRELRLRLDQLTANSARLEVERDNLAQDLATVRQKLQDETNLRLEAENNLAAYRQEADEATLARLDLERKIESLEEEIRFLRKIHEEEVRELQEQLARQQVHVELDVAKPDLTAALKEIRTQYEAMASSNMHEAEEWYRSKFADLTDAAARNAELLRQAKHEANDYRRQLQSLTCDLESLRGTNESLERQMREQEERHVREAASYQEALARLEEEGQSLKDEMARHLQEYQDLLNVKLALDIEIATYRKLLEGEENRITIPVQTFSNLQIRGGKSTKDGENHKVTRYLKSLTIRVIPIQAHQIVNGTPPARETSLDTKSVSEGHLKRNIVVKTVEMRDGEVIKESKQEHKDVM

(19) United States
(12) Patent Application Publication
(10) Pub. No.: US 2026/0097096 A1
(43) Pub. Date: Apr. 9, 2026

(54) 4-1BBL AND IL-12 THERAPY FOR TREATMENT OF GLIOBLASTOMA

(71) Applicant: The General Hospital Corporation, Boston, MA (US)

(72) Inventors: Koen Breyne, Reading, MA (US); Xandra O. Breakefield, Newton, MA (US); Thorsten R. Mempel, Chestnut Hill, MA (US)

(21) Appl. No.: 19/115,409

(22) PCT Filed: Sep. 27, 2023
(86) PCT No.: PCT/US2023/075225

§ 371 Date: Mar. 26, 2025

Related U.S. Application Data
(60) Provisional application No. 63/410,744, filed on Sep. 28, 2022.
(60) Provisional application No. 63/524,839, filed on Jul. 3, 2023.
(60) Provisional application No. 63/526,224, filed on Jul. 12, 2023.

(51) Int. Cl. A61K 38/17; A61K 38/20; A61K 48/00; A61P 35/00; C12N 15/86

(52) U.S. Cl. CPC A61K 38/177; A61K 38/208; A61K 48/005; A61P 35/00; C12N 15/86; C12N 2740/15043; C12N 2750/14143

(57) ABSTRACT

Provided herein are methods of treating glioblastoma including administering to a subject having glioblastoma a therapeutically effective amount of a pharmaceutical composition comprising 4-1BBL, optionally in combination with recombinant IL-12. The 4-1BBL can be provided to the subject via an adeno-associated virus, for example AAV-F, and the IL-12 can be provided by intratumoral injection.

### CLAIM OF PRIORITY

This application claims the benefit of U.S. Provisional Application Ser. No. 63/410,744, filed on Sep. 28, 2022, U.S. Provisional Application Ser. No. 63/524,839, filed on Jul. 3, 2023, and U.S. Provisional Application Ser. No. 63/526,224, filed Jul. 12, 2023. The entire contents of each of the foregoing are incorporated herein by reference.

### FEDERALLY SPONSORED RESEARCH OR DEVELOPMENT

This invention was made with Government support under Grant No. CA232103 awarded by the National Institutes of Health. The Government has certain rights in the invention.

### SEQUENCE LISTING

This application contains a Sequence Listing that has been submitted electronically as an XML file named “29539-0709WO1_ST26_SL.XML.” The XML file, created on Sep. 26, 2023, is 17,506 bytes in size. The material in the XML file is hereby incorporated by reference in its entirety.

### TECHNICAL FIELD

This disclosure describes a therapeutic compositions and treatments for glioblastoma.

### BACKGROUND

Glioblastoma (GB) is the most aggressive primary cancer in the central nervous system (CNS) (Molinaro et al., 2019). The standard of care is tumor resection followed by radiotherapy and temozolomide treatment (Stupp et al., 2005), with a median survival of 14.7 months after first diagnosis (van Solinge et al., 2022; Zhu et al., 2017). Compared to other human cancers, GB is generally considered a “cold” tumor with low levels of neoantigens, which restricts the generation of tumor-specific immunity (Segura-Collar et al., 2023). Although the intricacies of GB immunology are still being uncovered, it is believed that even when antitumor immune response is developed, it is blocked by the adaptability of both tumor cells and the tumor microenvironment (TME). Factors at the cellular, molecular, and genetic levels contribute to the immune suppressive nature of GB in patients (Broekman et al., 2018). Numerous clinical trials have tried to reinvigorate antitumor immunity in GB, which has proven effective in non-cranial tumors, by targeting immune checkpoint inhibitors (ICI), such as programmed cell death protein-1 (PD-1) (nivolumab and pembrolizumab), PD-L1 (atezolizumab and durvalumab), and T lymphocyte-associated antigen 4 (CTLA-4) (ipilimumab) (Cloughesy et al., 2019; Filley et al., 2017; Kurz et al., 2018; Nayak et al., 2021; Omuro et al., 2018; Reardon et al., 2020; Schalper et al., 2019). Unfortunately, therapeutic efficacy could not be demonstrated with these ICI strategies in GB (Tomaszewski et al., 2019).

### SUMMARY

Provided herein are methods of treating glioblastoma, the methods including administering to a subject having glioblastoma a therapeutically effective amount of 4-1BBL in combination with interleukin 12 (IL-12), optionally recombinant IL-12 (rIL-12). In some embodiments, the rIL-12 comprises a fusion protein of IL-12 conjugated to Fc. In some embodiments, the 4-1BBL comprises human 4-1BBL (h4-1BBL). In some embodiments, the 4-1BBL is administered one or more times. In some embodiments, the 4-1BBL is administered before, concurrently with, or after the IL-12. In some embodiments, the method of treating glioblastoma comprises administering to the subject a vector encoding 4-1BBL. In some embodiments, the vector is a viral vector. In some embodiments, the viral vector is a lentiviral vector. In some embodiments, the viral vector is an adeno-associated virus (AAV) vector. In some embodiments, the AAV vector is an AAV-F or an AAV-9 capsid. In some embodiments, the viral vector comprises a GFAP promoter. In some embodiments, administering the vector comprises intracranial or intratumoral administration. In some embodiments, the glioblastoma comprises primary glioblastoma or recurrent glioblastoma.

Provided herein is recombinant 4-1BBL and IL-12 composition for use in a method of treating glioblastoma. In some embodiments, the 4-1BBL is administered before, concurrently with, or after the IL-12. In some embodiments, the 4-1BBL is administered one or more times. In some embodiments, the 4-1BBL is administered alone, without IL-12. In some embodiments, the method of treating glioblastoma comprises administering to the subject a vector encoding 4-1BBL. In some embodiments, the vector is a viral vector. In some embodiments, the viral vector is a lentiviral vector. In some embodiments, the viral vector is an adeno-associated virus (AAV) vector. In some embodiments, the AAV vector is an AAV-F capsid or an AAV-9 capsid. In some embodiments, the viral vector comprises a GFAP promoter. In some embodiments, administering the vector comprises intracranial or intratumoral administration. In some embodiments, the glioblastoma comprises primary glioblastoma or recurrent glioblastoma.

Provided here are compositions including an AAV-F vector comprising a GFAP promoter operably linked to a sequence encoding 4-1BBL. In some embodiments, the 4-1BBL is human 4-1BBL. In some embodiments, the 4-1BBL is mouse 4-1BBL.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages will be apparent from the following detailed description and figures, and from the claims.

### DESCRIPTION OF DRAWINGS

FIG. 1A is a schematic illustration of the in vivo experimental set-up. CT-2A-Firefly (Fluc) glioma (100,000) cells were injected intracranially (i.e.) into the left striatum on day 0. Starting on day 7, tumour growth was monitored every 3 to 4 days by IVIS bioluminescence imaging. Based on Fluc levels, mice with a similar tumor sizes were allocated to sham (Fc control) or rIL-12 (recombinant IL-12 conjugated to Fc)-treatment groups (ranging between 5 to 500 ng) on day 10. To ensure intratumoral exposure to the treatment, sham and rIL-12 solutions were administered to the same i.e. injection site used to inoculate the tumor cells.

FIG. 1B is a depiction of regression of tumors in GB-bearing mice with rIL-12 over time. Fluc readings demonstrate that tumors were established in the brains of mice (n=5-11 mice/group) at time of i.e. treatment (sham or rIL-12) on day 10. Different outcomes were observed on day 18 and 22 post tumor-implantation depending on the rIL-12 dose that GB-bearing mice received. The shown images are representative IVIS images of GB-bearing mice from each treatment condition and “+” indicates that animals died prior to the imaging timepoint.

FIG. 1C is a chart documenting the survival benefit of GB-bearing mice with rIL-12 treatment. Kaplan-Meier survival curves shows rIL-12 dose-escalation study in mice (n=5-11 mice/group) with glioma tumors, compared with sham. Arrow indicates time of i.e. injection of sham or rIL-12. The median survival was significantly increased compared to sham if GB-mice were treated with either 50 ng, 200 ng, and 500 ng (27, 38, and 34 days respectively). Some mice in the 200 ng and 500 ng groups stayed healthy over 60 days prior to being sacrificed for neuropathology analysis.

FIG. 1D show tumor sizes in brains of rIL-12 treated GB-bearing mice. Brain sections of mice 22 days post—i.e. implantation with CT-2A-Fluc and treatment with sham or rIL-12 were stained for hematoxylin and eosin (H&E) (4× magnification, scale bar=5 μm). The black dotted line indicates the tumor border.

FIG. 1E shows differences in CT-2A-Fluc bioluminescence levels in GB-bearing mice at day 7 post-tumor implantation, prior to i.e. treatment. No significant differences were observed between groups (50 ng sham, n=7:5 ng rIL-12, n=6:20 ng rIL-12, n=5:50 ng rIL-12, n=11:200 ng rIL-12, n=5, and 500 ng rIL-12, n=6).

FIG. 1F shows classification of rIL-12 treated GB-bearing mice. Based on tumor growth and weight loss, three categories could be distinguished. Non-responders (n=27) had similar results as the sham treatment. Treatment-responders (n=9) performed better than the sham-treated mice, but still died. Survivors (n=4) had visible tumor regression due to treatment. In the left graphs, CT-2AFluc-bearing mice treated rIL-12 or sham were monitored every 3-4 days by IVIS imaging, which is representative of the tumor size in the brain. Dotted line represents the background signal. In the middle graphs, the weight of the mice was tracked over time. TO represents the weight at start of the experiment. The pie graphs on right show the percentage of mice that are allocated to a certain category based on survival.

Data represents at least two independent experiments. Data were analyzed using Log-rank (Mantel-Cox) test using Graph Pad Prism 9.5.1, **p<0.01, ****p<0.0001 for FIG. 1 data.

FIG. 2A shows schematics and expression of IL-12 receptor beta (B) in immune cells populations. Single cell RNA sequencing (scRNAseq) datasets of CD45-sorted cells derived from mouse GB tumor (GL261, n=3), human primary GB tumor (n=7) and human recurrent GB tumor (n=4) were analyzed (left). Distinct cell type subsets were clustered, annotated and visualized with a high-resolution color coded UMAP projection (middle). To visualize IL-12rb1 and IL-12rb2 expression in different datasets, single cell violin plots are used to compare transcript levels in Mo/Mφ cells (TAM, proliferative TAM and monocytes), dendritic cell cells (DC1, DC2, DC3 and DC4), NK/T cell cells (reg T cells, NK cells (A-D), T cells and other cell cluster (B cells, plasma B cells, mast cells). Dataset was acquired from Pombo Antunes et al., 2021 and analyzed with R and Seurat.

FIG. 2B shows CD11b expression in the Mo/Mφ cluster. In the scRNAseq dataset of GB-bearing mice, expression of Ptprc (CD45) was observed in the Mo/Mφ cells cluster, dendritic cells cluster, NK/T cell cluster and other cells cluster while Itgam (CD11b) expression was only visible in the Mo/Mφ cluster.

FIG. 2C shows decoupling Il-12 receptor expressing Mo/Mφ cells from other immune cells in GB-bearing mouse brains. A schematic display shows the sequential method used to fractionate Mo/Mφ and non-Mo/Mφ immune cells with tumor hemisphere (TH) cell fractions enriched for anti-CD11b and anti-CD45 post-enzymatic treatment of mouse brains to generate single cells. Non-immune cells can also be analyzed as they flow through both columns.

FIG. 2D shows eight days after rIL12-injection, Il12rb1/2 expression in each GB-brain fraction. Eight days after rIL12-injection, Il12rb2 was expressed at significantly higher levels in CD11b^{NEG} immune cells compared to CD11b^{POS} and non-immune cells. Il12rb1 was not significantly different between the different fractions. Data represent CT values normalized to β-actin.

FIG. 2E shows CD8 T cells on tumor border express IL-12 receptor. CD8^{POS} CTLs were present at the (CT-2A) tumor border (dotted white line) (top, scale bar=10 μm). CD8^{POS} T cell expressed IL12rb1 (bottom, scale bar=50 μm) 18 days post-tumor implantation.

FIG. 2F shows reactive CTLs analyzed in CD11b^{NEG} fraction of GB-mouse brain. Eight days after rIL12-injection, IFN-γ (middle) was expressed at significantly higher levels in CD11b^{NEG} immune cells compared to CD11b^{POS} and non-immune cells. Data represent Ct values normalized to β-actin.

FIG. 2G shows Cd11b mRNA expression in immune cell subsets. Eight days after rIL12-injection, Cd11b expression was analyzed in specific cell fractions from GB-bearing brains. CD11b was expressed at significantly higher levels in CD11b^{POS} immune cells fractions as compared to CD11b^{NEG} immune cells and non-immune cells in both treatment conditions. Data represents Ct values normalized to β-actin.

FIG. 2H shows expression of interferon gamma (Ifn-g) in immune cells populations. scRNAseq cluster datasets of CD45-sorted cells derived from mouse GB tumor (GL261, n=3), human primary GB tumor (n=7) and human recurrent GB tumor (n=4) were analyzed (left).

FIG. 2I shows distinct cell type subsets were clustered, annotated and visualized for Il12rb1, Il12rb2 and Ifng with a high-resolution UMAP projection in mouse GB, human primary GB and human recurrent GB. Dataset was acquired from Pombo Antunes et al., 2021 and analyzed with R and Seurat.

FIG. 2J shows a percentage of positive cells in each immune cell cluster is shown by bar graphs. Datasets acquired were analyzed for Il12rb1, Il12rb2 and Ifng in mouse GB, human primary GB and human recurrent GB, including Mo/Mφ cells, NK—natural killer cells and DC—dendritic cells.

Data represent at least three independent experiments and are presented as the mean with SEM (error bars), Data were analyzed using one-way ANOVA test using Graph Pad Prism 9.5.1*p<0.05, **p<0.01, ***p<0.001, ****p<0.0001 for FIG. 2 data.

FIG. 3A shows depletion of CD8^{POS} cells at tumor site. Schematic illustration of the T cell depletion strategy (top). At day 0, 100,000 glioma cells (CT-2A-Fluc) were implanted i.e. into the left striatum. Anti-CD8 or IgG control was injected i.v. at day 9 (50 μg). At day 10, mice were injected with 50 ng rIL-12 or sham (Fc) control i.e. at the tumor site and anti-CD8 or IgG control was injected i.v. (100 μg) to deplete endogenous CD8^{POS} T-cells systemically. Validation of successful T cell depletion in brain was shown by the absence of CD8^{POS} cells in representative flow cytometry plots (bottom) after treatment with anti-CD8. (TU-Tumor).

FIG. 3B shows the importance of CD8^{POS} T cell recruitment for survival benefit in anti-GB therapy with rIL12. Kaplan-Meier curves showing survival outcome of tumor-bearing mice injected without CD8-depletion (IgG) and rIL-12, with CD8 depletion (anti-CD8) and rIL-12 without CD8 depletion (IgG) and no treatment, and with CD8-depletion (anti-CD8) and sham (n=6-8 mice/group). IgG control treated with rIL-12 had a median survival of 25 days, whereas other groups had a median survival of 20 days.

FIG. 3C shows an increase of CD8^{POS} T cells at tumor site with intratumoral rIL-12. Representative flow cytometry plots show the gating for live cells based on uptake of the viability dye ZombieBlue staining. Isolation of Thy 1.2^{POS} and CD8^{POS} T cells, pre-gated for CD45^{POS}CD11b^{NEG} in brains of tumor-bearing mice on day 18 post-tumor implantation, comparing rIL-12 treated and sham control (left). The brain tissue was enzymatic digested, depleted of CD11b^{POS} cells and enriched for CD45^{POS} cells. Bar graph represents quantification of CTL numbers in different brain hemispheres of mice (n=3 mice per group) as a percentage of single cells in rIL-12/sham treated mice (right, bar graph).

FIG. 3D shows differentiating stem- and effector-like CTLs at mouse GB tumor site. scRNAseq analysis distinguishes NK cells from T cells based on Klrb1c and Cd3b expression, respectively. Then, helper and regulatory T cells, expressing Cd4 and Foxp3 genes, were discriminated from the CTLs by marked Cd8a b expression. In the CTL cluster, we observed naïve and stem-like CTLs, expressing Tcf7 (encoding TCF-1) and Nsg2 genes, that were different from the CTLs with an effector-like phenotype expressing Havcr2 (encoding TIM-3), Pdcd1 (encoding for PD-1), Gzmb (encoding for cytotoxic granzyme-B) and Tnfrsf9 (encoding for 4-1BB).

FIG. 3E shows increased CTL differentiation upon rIL-12 treatment of GB. Overlaid contour plots of TCF-1 expression against TIM-3^{POS} comparing rIL-12 and Fc control (left). Quantification of the percentage of CTLs comparing TCF-1^{POS} (panel 1), TCF-1^{NEG}TIM-3^{NEG} (panel 2) and TIM-3^{POS} (panel 3) showed a significant increase of TIM-3^{POS} after rIL-12 treatment compared to sham (n=3 mice per condition, right bar graph).

FIG. 3F shows PD-1 differentiation marker is highly expressed in effector-like CTLs. Percent of maximum PD-1 expression within TCF-1^{POS} (panel 1), TCF-1^{NEG}TIM-3^{NEG} (panel 2) and TIM-3^{POS} (panel 3) populations comparing rIL-12 and sham control. Panel numbers correspond to numbers in E. Quantification of flow cytometry PD-1^{POS} comparing rIL-12 and sham within TCF-1^{POS} (panel 1), TCF-1^{NEG}TIM-3^{NEG} (panel 2) and TIM-3^{POS} (panel 3) populations. TIM-3^{POS} cells express significantly more PD-1 compared to TCF-1^{POS} and TIM-3^{NEG}TCF-1^{NEG} cells. In contrast, TIM-3^{NEG}TCF-1^{NEG} CTLs express significantly more PD-1^{POS} cells, compared to TCF-1^{POS} ones.

FIG. 3G shows cytotoxic GZM-B is highly expressed in effector-like CTLs. Quantification of flow cytometry of GZM-B comparing rIL-12 and sham control within TCF-1^{POS} (panel 1), TCF-1^{NEG}TIM-3^{NEG} (panel 2) and TIM-3^{POS} (panel 3) populations.

FIG. 3H shows validation of CD8^{POS} T cell depletion shown by the absence of CD8^{POS} cells in representative flow cytometry plots showing cell fractions enriched for CD11b or CD45, pre-gated for CD45^{POS} cells, after treatment with anti-CD8 compared to IgG control for brain and spleen samples.

FIG. 3I shows gene expression levels in retro-orbital blood samples obtained from CD8-depleted mice show a significant drop in CD8b at day 11 and 18, post CD8-depletion; no significant drop of CD8 was observed at day 7 (prior to CD8-depletion).

FIG. 3J shows tumor growth and weight were measured over time in tumor-bearing mice injected with IgG and rIL-12, anti-CD8 and rIL-12, IgG and Fc control, and anti-CD8 and Fc control. After T-cell depletion, mice had increased tumor sizes. Weights of all mice dropped starting day 14 after tumor cell injection.

FIGS. 3K&L show differentiating stem- and effector-like CTLs at human primary and recurrent GB tumor site. scRNAseq analysis distinguishes NK cells from T cells based on KLRB1 and CD3E expression, respectively. Then, helper and regulatory T cells, expressing Cd4 and Foxp3 genes, were discriminated from the CTLs marked by CD8A/B expression. In the CTL cluster, we observed naïve and stem-like CTLs, expressing TCF7 (encoding TCF-1) and HMP19 genes, that were different from the CTLs with an effector-like phenotype expressing HAVCR2 (encoding TIM-3), PDCD1 (encoding PD-1), GZM-B (encoding cytotoxic granzyme-B) and TNFRSF9 (encoding 4-1BB).

FIG. 3M shows a schematic overview to illustrate the stages of T cell differentiation.

FIG. 3N shows gene expression levels showed that Pdcd1 (the PD-1 gene), Gzmb, and Cd-101 transcript were expressed at significantly higher levels in rIL-12 treated compared to Fc control measured in RNA from total mouse brain, while Tcf7 (the TCF-1 gene) and Havcr2 (encoding TIM-3) markers were not significantly different.

FIG. 3O shows Quantification of flow cytometry PD-1_{POS} and PD-1_{NEG} comparing rIL-12 and sham control within TCF-1_{POS} (panel 1), TCF-1_{NEG}TIM-3_{NEG} (panel 2) and TIM-3_{POS} (panel 3) cell populations. TIM-3_{POS} cells expressed significantly higher PD-1 compared to TCF-1_{POS} and TIM-3_{NEG}TCF-1_{NEG} cells. TIM-3_{POS} cells expressed significantly less PD-1 compared to TCF-1_{POS} and TIM-3_{NEG}TCF-1_{NEG} cells. Data represent two independent experiments and are presented as the mean with SEM (error bars). Data were analyzed using unpaired t test in C, two-way ANOVA in E and F, and Log-rank (Mantel-Cox) test for survival using Graph Pad Prism 9.5.1, *p<0.05, **p<0.01, ***p<0.001, ****p<0.0001 for FIG. 3 data.

FIG. 4A shows IL-12 expressing cells in the TME of GB. High numbers of YFP expressing cells were observed in both the tumor border as well as the tumor itself in IL-12b-YFP reporter mice treated with 50 ng rIL-12. Boxed areas on left are viewed at 10× magnification (scale bar=10 μm) and higher magnification on right (40× magnification, scale bar=50 μm).

FIG. 4B shows IL-12 expressed by myeloid and non-myeloid TME cells of GB. IL-12b was expressed at significantly higher levels in the CD11b^{POS} and CD11b^{NEG}CD45^{POS} immune cells as compared to the non-immune cells, while IL-12a was expressed at similar levels in all subsets.

FIG. 4C shows IL-12 expressed by DC cluster of non-myeloid TME cells of GB. scRNAseq showing expression levels of IL12b expression in mouse GB, primary human GB or recurrent human GB in Mo/Mφ cells and DCs. A lack of IL-12b was seen for both primary and recurrent GB patients compared to the GB mouse model.

FIG. 4D shows lack of IL-12 does not influence survival of GB mice. Kaplan-Meier survival curve shows survival outcomes of tumor-bearing IL-12^{-/-} mice (dashed line) and IL-12^{+/+} mice (solid line) injected intracranially with 50 ng rIL-12 or sham (black) (n=4-6 mice per group. All mice injected with rIL-12 had a median survival of 25 days, whereas sham had 20 days median survival.

FIG. 4E shows IL-12 expression restricted to subset of DCs. UMAP clustering shows expression of IL12b in distinct population of the DC cluster.

FIG. 4F shows CD11c^{POS} IL-12 expressing DCs are recruited to the GB TME. DCs were stained for CD11c and express YFP (10× magnification, scale bar=10 μm; 40× magnification, scale bar=50 μm).

FIG. 4G shows IL-12 expression profile matches CCR-7^{POS}-DCs. IL12b was highly expressed in subcluster CCR-7^{POS}_DCs which had marked expression of Fsn1, Ccr7 and Ccl22.

FIG. 4H shows visualization of CCR-7^{POS}-DCs in scRNAseq dataset. The cells positive in panel E match with the dendritic specific markers Fscn1, Ccr7, and Ccl22.

FIG. 4I shows regulatory factors of interest for CTL differentiation by CCR-7^{POS}_DCs. Heatmap showing co-expression of genes that are generated by CCR7^{POS}-DCs.

FIG. 4J shows 4-1BB+ CTLs are effector-like CTLs. Quantification by flow cytometry of 4-1BB comparing rIL-12 and Fc control within TCF-1^{POS} (panel 1), TCF-1^{NEG}TIM-3^{NEG} (panel 2) and TIM-3^{POS} (panel 3) populations. (n=4-6 mice per group).

FIG. 4K shows 4-1BB CTLs are recruited at the tumor upon IL-12 treatment. Immunohistochemistry shows that 4-1BB was expressed in CD8^{POS} CTLs post-rIL-12 treatment at the tumor border. (40× magnification, scale bar=50 μm).

FIG. 4L shows 4-1BB effector-like CTLs express IL-12R and have GZM-B activity. Overlaid contour plots of 4-1BB expression versus GZM-B in TIM-3^{POS} PD-1^{POS} CTLs comparing rIL-12RB2^{NEG} (black) and rIL-12RB2^{POS} showing: 4-1BB^{NEG}GZM-B^{NEG} (panel 1), GZM-B^{POS} (panel 2), 4-1BB^{POS} (panel 3), 4-1BB^{POS}GZM-B^{POS} (panel 4). There was no significant difference observed in 4-1BB and/or GZM-B expression in Fc treated groups, however the percentage of 4-1BB^{POS}GZM-B^{POS} (panel 4) was significantly increased in TIM-3^{POS} PD-1^{POS} CTLs after rIL-12 treatment. (n=4-6 mice per group).

FIG. 4M shows scRNAseq showing expression levels of IL12a expression in mouse GB, human GB or recurrent human GB in Mo/Mφ, NK/T, DC or other clusters (NK/T—Natural killer/t cells, DC—dendritic cells, and B cells, plasma b cells and mast cells).

FIG. 4N shows bioluminescence imaging showing tumor growth in Il12b−/− mice (dashed line) and Il12b+/+ mice (solid line) injected intracranially with 50 ng rIL-12 or sham. All mice injected with sham or rIL-12 had a tumor of 10e8 bioluminescence signal. Upper line graph is sham treated, whereas bottom line graph is rIL-12 treated.

FIGS. 4O&P show a heatmap showing expression of immune related genes in Mo/Mφ, dendritic cell and NK/T cell clusters in human primary GB and human recurrent GB, respectively.

FIGS. 4Q&R show heatmaps showing co-expression of CCR7POS DC cluster genes identified in Mo/Mφ, dendritic and NK/T cells clusters and other cell clusters in human primary GB and human recurrent GB.

FIG. 4S shows overlaid counter plots of 4-1BB expression within the PD-1^{POS} TIM-3^{POS} and PD-1^{POS} TIM-3^{NEG} populations showed the increased presence of 4-1BB receptor in the hemisphere ipsilateral to the tumor compared to the contralateral hemisphere and the spleen.

FIG. 4T shows a bar graph shows MFI plots of Il12rb expression in Tim3^{POS} and PD-1^{POS} CTLs comparing sham and rIL-12 treatment showing: 4-1BB^{NEG} GZM-B^{NEG} GZM-B^{POS}, 4-1BB^{POS}, 4-1BB^{POS}, GZM-B^{POS} cell clusters. There was a significant difference observed in negative vs 4-1BB^{POS} GZM-B^{POS} cells in sham treated group, and Il12rb2 expression was significantly increased in 41BB^{POS} and 4-1BB^{POS} GZM-B^{POS} CTLs populations after rIL-12 treatment.

Data represents two independent experiments and are presented as the mean with SEM (error bars). Data were analyzed using two-way ANOVA, Log Rank (Mantel-Cox) test for survival using Graph Pad Prism 9.5.1, *p<0.05, **p<0.01, ***p<0.001, ****p<0.0001 for FIG. 4 data.

FIG. 5A shows GB tumors in mice express high levels of PD-L1. Immune (CD11b^{POS}, CD11b^{NEG}) and non-immune cell populations were isolated from tumor-bearing region of brains using CD11b beads and CD45 beads. Cd274 (the PD-L1 gene) was expressed at significantly higher levels in non-immune cells. No differences were observed between Fc control and rIL12 treatment. Gene expression levels were normalized to β-actin. (n=4 mice per group).

FIG. 5B shows lentiviral m4-1BBL constructs. Schematic display of m4-1BBL construct containing mCherry labelled m4-1BBL and 3×FLAG-tag (CT-2A-Fluc-m4-1BBL) and the control construct lacking m4-1BBL (CT-2A-Fluc-null), both driven by a GFAP promotor.

FIG. 5C shows m4-1BBL expression in GB mouse cells. CT-2A transduced with the 4-1BBL LVV showed significant enhanced gene expression levels of TNfsf9 (encoding for 4-1BBL) compared to non-transduced WT CT-2A and the control construct, normalized to β-Actin. (n=3 biological replicates).

FIG. 5D shows m4-1BBL protein expression in GB mouse cells. 3×FLAG-tag protein levels (37.5 kDa) were only present in CT-2A cells transfected with the CT-2A-Fluc-m4-1BBL construct normalized to β-Actin. 3×FLAG-tag detection enabled detection of transgene m4-1BBL and not endogenous 4-1BBL.

FIG. 5E shows homogenous m4-1BBL expression in transduced GB mouse cell line. Immunohistochemistry images of m4-1BBL overexpressing CT-2A cells (CT-2A-Fluc-m4-1BBL) in culture stained for DAPI 3×FLAG-tag, mCherry and 4-1BBL with a merged image (scale bar=50 μm).

FIG. 5F shows experimental outline to test local expression of m4-1BBL and rIL-12 treatment. The in vivo approach is schematically displayed; CT-2A-Fluc-null or CT-2A-Fluc-m4-1BBL were implanted i.e., mice were treated with rIL-12 or sham (PBS or Fc-control) 10 days after tumor injection.

FIG. 5G shows survival benefit of local 4-1BBL expression post-rIL-12 treatment. Kaplan-Meier curves showing survival outcomes following treatment of CT-2A-Fluc-control with rIL-12 (solid grey) or sham (dashed grey), and CT-2A-Fluc-m4-1BBL treated with rIL-12 or sham (n=5-11 mice/group). Tumor cells expressing the control vector, lacking m4-1BBL, showed poor survival outcome, and survival was significantly improved after treatment of tumor cells expressing m4-1BBL with rIL-12 (50 ng). Mice injected with CT-2A-Fluc-m4-1BBL tumor cells treated with rIL-12 showed significantly increased survival compared to sham or IL-12 treatment only.

FIG. 5H shows confirmation of m4-1BBL transgene expression at tumor site. Immunohistochemistry of CT-2A-Fluc-m4-1BBL tumor-bearing mouse brains confirmed transgene expression (mCherry-positive cells) co-localized with 3×FLAG-tag and m4-1BBL. (40× magnification, scale bar=50 μm).

FIG. 5I shows GB mouse survival upon m4-1BBL and rIL-12 combination treatment is not dependent on endogenous IL-12. Kaplan-Meier curves of Il12^{-/-} and I/12^{+/+} mice showing survival outcome of CT-2A-Fluc-m4-1BBL tumor-bearing mice injected intratumorally with rIL-12, or the sham. Mice (n=9-12 mice per genotype) treated with rIL-12 stayed alive for more than 31 days, while sham treated mice had a median overall survival of 25 days.

FIG. 5J shows experimental outline to test CD8 T cell dependency of m4-1BBL and rIL-12 combination treatment. Schematic display shows i.v. injection with or without CD8 T cell depletion (anti-CD8 or IgG control, respectively) at day 9 and day 10 (50 μg and 100 μg at day 9 and 10, respectively) post-CT-2A-Fluc-m4-1BBL intracranial tumor implantation. Mice were injected i.e. with rIL-12 (50 ng) on day 10.

FIG. 5K shows GB mouse survival benefit of m4-1BBL and rIL-12 combination treatment is CD8 T cell dependent. Kaplan-Meier curves of a total of 12 Il12b^{+/+} mice showing survival outcomes of CT-2A-Fluc-m4-1BBL tumor-bearing mice all treated with rIL-12, after treatment with anti-CD8 or IgG control. Mice (n=5-6 mice per group) treated with IgG control had a median overall survival of 23.5 days, compared to 20 days for mice treated with anti-CD8.

FIG. 5L shows GB mouse survival benefit due to CD8 T cell recruitment of m4-1BBL, and rIL-12 combination treatment is not dependent on endogenous IL-12. Kaplan-Meier curves of a total of 12 Il12b^{-/-} mice showing survival outcomes of CT-2A-Fluc-m4-1BBL tumor-bearing mice all treated with rIL-12, after treatment with anti-CD8 or IgG control. Mice (n=5-6 mice per group) treated with IgG control had a median overall survival of 35 days, compared to 21 days for mice treated with anti-CD8.

FIG. 5M shows weight (left) and tumor growth (right) were measured over time in WT mice injected with CT-2A-Fluc-control or CT-2A-Fluc-m4-1BBL tumor cells comparing rIL-12 (solid) to sham control (dashed) treatment (n=5-11 mice per group).

FIG. 5N shows weight (left) and tumor growth (right) were measured over time in 1112b−/− mice injected with CT-2A-Fluc or CT-2A-Fluc-m4-1BBL tumor cells and treated with rIL-12 or sham. Mice treated with Fc control showed a weight drop at day 22, while rIL-12 treated mice maintained their weight over 50 days. Mice treated with rIL-12 showed a decrease in tumor size starting at day 22, with increasing size in Fc treated mice to day 28-time of death (n=9-12 mice per group).

FIG. 5O shows weight (left) and tumor growth were measured over time in tumor-bearing Il12b+/+ mice injected with IgG and rIL-12 or anti-CD8 and rIL-12. After T-cell depletion, weights of mice dropped at day 21 and these mice had significantly increased tumor sizes compared to IgG control. (n=5-6 mice per group).

FIG. 5P shows weight (left) and tumor growth (right) were measured over time in tumor-bearing Il12b−/− mice injected with IgG and rIL-12 or anti-CD8 and rIL-12. After T-cell depletion, weights of mice dropped at day 14 and these mice had significantly increased tumor sizes compared to IgG control. (n=5-6 mice per group). Data represent at least two independent experiments and are presented as the mean with #SEM (error bars). Data were analyzed using one-way ANOVA and Log rank (Mantel-Cox) test for survival using Graph Pad Prism 9.5.1. *p<0.05, **p<0.01, ***p<0.001, ****p<0.0001 for FIG. 5 data.

FIG. 6A shows AAVF vector constructs to deliver m4-1BBL to tumor site. Schematic representation of the 4-1BBL AAVF (AAVF-GFAP-m4-1BBL) and control AAVF (AAVF-GFAP-null) constructs. The m4-1BBL-3×FLAG-tag and m4-1BBL are under a GFAP promotor with a poly(A) signal after the coding sequence. In the control AAVF-GFAP-null, the GFAP promotor and poly(A) signal were connected without the presence of intervening sequences.

FIG. 6B shows m4-1BBL protein expression at tumor site. 3×FLAG-tag protein was only detected in brains injected with AAVF-GFAP-m4-1BBL-3×FLAG-tag (37.5 kDa) as normalized to β-Actin (42 kDa) by western blot analysis. No fragmentation of the transgenic product was observed.

FIG. 6C shows graphic depiction of the treatment scheme of AAVF-GFAP-m4-1BBL experiments. m4-1BBL-coding or control AAVF vectors were injected intratumorally at three time points; one day prior to tumor implantation, at the time of tumor implantation, and 1-day post-tumor implantation. rIL-12 or sham were injected intracranially at day 10 post-implantation at the tumor site, and mice were followed by IVIS every 4 days.

FIG. 6D shows survival benefit with AAV-mediated delivery of m4-1BBL in rIL-12 treated GB-bearing mice. Kaplan-Meier curves displaying the percentage of survival of CT-2A-Fluc-bearing mice (12,500 cells at the time of injection) comparing AAVF-GFAP-m4-1BBL and AAVF-GFAP-null (black) vectors both treated with rIL-12 (n=4-6 mice per group). AAVF-GFAP-m4-1BBL rIL-12 treated had a median survival of 33.5 days compared to sham with a median survival of 19 days.

FIG. 6E shows m4-1BBL transgene expression in GFAP^{POS} cells at the tumor site after AAV-mediated delivery. Immunohistochemistry images of tumor-bearing mouse brains treated with AAVF-GFAP-m4-1BBL (top) or AAVF-null (bottom) vectors, and 50 ng rIL-12, stained for DAPI, GFAP and 4-1BBL. The white dotted line represents the tumor border. (40× magnification, scale bar=50 μm).

FIG. 6F shows endogenous GFAP expression in different mouse GB cell lines and astrocytes. RT-qPCR analysis measuring Gfap expression levels for glioma cell lines, CT-2A and 005, and primary brain-derived astrocytes. (n=6 per condition)

FIG. 6G shows GFAP^{POS} cell association in tumor is dependent on brain-implanted mouse GB cell line. Immunohistochemistry showing GFAP^{POS} astrocytes at the CT-2A tumor border (left) and 005-Fluc tumor border (right), 18 days post-implantation. With 005 cells the GFAP^{POS} cells were retrieved in the brain tumor cell mass. (4× magnification, scale bar=5 μm).

FIG. 6H shows survival of rIL-12 treated 005-Fluc-bearing mice. Kaplan-Meier curves displaying the percentage of survival of 005-Fluc-bearing mice (100,000 cells at the time of injection) with treatment at day 10 post-tumor comparing i.e. injection of 50 ng rIL-12 to sham control (Fc-black) (n=5-6 mice/group).

FIG. 6I shows survival benefit is reduced by delayed treatment of 005-Fluc-bearing mice with rIL-12. Kaplan-Meier curves displaying the percentage of survival of 005-Fluc-bearing mice (100,000 cells at the time of injection) with treatment at day 20 post-tumor implantation, comparing i.e. injection of 50 ng rIL-12 to sham control (black) (n=4-5 mice/group). No significant difference was observed for rIL-12 treated mice with a median survival of 35 days compared to sham with a median survival of 38 days.

FIG. 6J shows recovery of survival benefit with AAV-mediated delivery of m4-1BBL into delayed rIL-12 treatment of 005-Fluc-bearing mice. Kaplan-Meier curves displaying the percentage of survival of 005-Fluc-bearing mice (50,000 cells at the time of injection) comparing AAVF-GFAP-m4-1BBL and AAVF-GFAP-null (black) vectors both treated with rIL-12 on day 20 post-tumor implantation (n=5 mice/group). AAVF-GFAP-m4-1BBL rIL-12 treated had a median survival of >60 days compared to sham with a median survival of 33 days.

FIG. 6K shows both primary derived astrocytes CT-2A cells and 005 cells were transduced with AAVF-GFAP-m4-1BBL or AAVF-GFAP-null control and maintained for 7 days in culture. mRNA levels showed increased GFAP expression in astrocytes compared to CT-2A and 005 cells; all three cell types showed increased levels of the m4-1BBL transgene only after incubating with AAVF-GFAP-m4-1BBL, and not with the AAVF-GFAP-null control, compared to PBS control. Data are plotted as CT values normalized to β-actin.

FIG. 6L shows gene expression levels shown for Gfap, Il12a, Il12b, Il12rb1, Il12rb2 41bb and 41bbl mRNA measured in primary mouse astrocytes. Data are plotted as CT values normalized to β-actin and displayed as a heatmap.

FIG. 6M shows immunohistochemistry of brain sections from mice implanted with CT-2A tumor cells and i.e. injected with AAVF-GFP backbone vector showed successful targeting of GFAP astrocytes after 14 days post-injection in the tumor vicinity. (10× magnification, scale bar=10 μm, left; 40× magnification, scale bar=50 μm, right).

FIG. 6N Kaplan-Meier curves displaying the percentage of survival of CT-2A-Fluc-bearing mice (100,000 cells injected) comparing AAVF-GFAP-null+sham (dashed black); AAVF-GFAPnull+rIL-12; (solid black); AAVF-GFAP-m4-1BBL+sham; AAVF-GFAPm4-1BBL+rIL-12 (n=5 mice in each group). AAVF-GFAP-null and AAVFGFAP-m4-1BBL were injected i.e. at the time of CT-2A tumor cell implantation. AAVFGFAP-m4-1BBL sham and rIL-12 treated had a median survival of 28 and 38 days, respectively, whereas AAVF-GFAP-null, sham and rIL-12 treated had a median survival of 25 and 37 days, respectively. Weight loss and average bioluminescence for tumor-bearing mice was measured over time comparing AAVF-GFAP-null sham (dashed black); AAVF-GFAPnull+rIL-12; AAVF-GFAP-m4-1BBL sham; AAVF-GFAP-m4-1BBL+rIL-12 (n=5 mice each group).

FIG. 6O shows immunohistochemistry of brain sections from mice implanted with CT-2A tumor and injected i.e. with AAVF-GFAP null and AAVF-GFAP-m-4-1BBL vector showed successful targeting of GFAP astrocytes and 3×FLAG-tag in TME in the tumor vicinity. The white dotted line represents the tumor border. (20× magnification, scale bar=50 μm).

FIG. 6P shows Kaplan-Meier curves displaying the percentage of survival of CT-2A-Fluc-bearing mice (12,500 cells at the time of injection) comparing AAVF-GFAP-null sham (dashed black); AAVF-GFAP-nullm+ rIL-12; (solid black); AAVF-GFAP-m4-1BBL+rIL-12 (n=4-6 mice in each group). AAVF-GFAP-null and AAVF-GFAP-m4-1BBL were injected i.e. three times, one day prior to tumor cell implantation, one at the time of tumor cell implantation, and one a day later after tumor cell implantation.

AAVF-GFAP-m4-1BBL+rIL-12 treatment had a median survival of 33.5 days as compared to AAVF-GFAP-null sham+rIL-12 treatment which had 24- and 19-days median survival, respectively. Weight loss and average bioluminescence for tumor-bearing mice was measured over time comparing AAVF-GFAPnull sham (dashed black); AAVF-GFAP-null+rIL-12; (solid black); AAVF-GFAP-m4-1BBL+rIL-12 (n=4-6 mice each group).

FIG. 6Q shows immunohistochemistry of brain sections from mice implanted with CT-2A tumor (12,500 cells) and i.e. injected with AAVF-GFAP null and m-4-1BBL vector (three times) showed successful targeting of GFAP astrocytes and 3×FLAG-tag in TME in the tumor vicinity. The white dotted line represents the tumor border. (40× magnification, scale bar=50 μm).

FIG. 6R shows Kaplan-Meier curves displaying the percentage survival of 005-Fluc-bearing mice (50,000 cells implanted) comparing AAVF-GFAP-null sham (dashed black); AAVF-GFAP-null+rIL12; (solid black); AAVF-GFAP-m4-1BBL sham, AAVF-GFAP-m4-1BBL+rIL-12 (n=4-5 mice in each group). AAVF-GFAP-null and AAVF-GFAP-m4-1BBL were intracranially injected three times, one day prior to tumor cell implantation, one at the time of tumor cell implantation, and one a day after tumor cell implantation. AAVF-GFAPm4-1BBL sham and rIL-12 treated had a median survival of 64.5 and 76 days, respectively, as compared to AAVF-GFAP-null sham and rIL-12 treated which had 42- and 35-days median survival, respectively. Weight loss and average bioluminescence for tumor-bearing mice was measured over time comparing AAVF-GFAP-null sham (dashed black); AAVF-GFAP-null+rIL-12; AAVF-GFAP-m4-1BBL sham AAVF-GFAP-m4-1BBL+rIL-12 (n=4-5 mice each group).

FIG. 6S shows GFAP^{POS} cells co-colocalize with 005-GFP tumor cells. Immunohistochemistry showing GFAP^{POS} astrocytes at the 005-GFP tumor border 18 days post-implantation. With 005 cells the GFAP^{POS} cells were retrieved in the brain tumor cell mass and co-localize with GFP. The white dotted line represents the tumor border. (4× magnification, scale bar=5 μm).

FIG. 6T shows immunohistochemistry of brain sections from mice implanted with 005-GFP tumor (12,500 cells) and i.e. injected with AAVF-GFAP null and m-4-1BBL vector (three times) showed successful targeting of GFAP astrocytes and 3×FLAG-tag (white) in TME in the tumor vicinity. The white dotted line represents the tumor border. (40× magnification, scale bar=50 μm). Data represent at least two independent experiments and are presented as the mean with ±SEM (error bars). Data were analyzed using one-way ANOVA and Log rank (Mante-Cox) test for survival using Graph Pad Prism 9.5.1. *p value=<0.05, **p<0.01, ***p<0.001 for FIG. 6 data.

### DETAILED DESCRIPTION

### Definitions

Unless specifically defined otherwise, all technical and scientific terms used herein shall be taken to have the same meaning as commonly understood by one of ordinary skill in the art (e.g., in cell culture, molecular genetics, and biochemistry).

As used herein, the singular forms “a,” “an,” and “the” include the plural reference unless the context clearly dictates otherwise. Thus, for example, a reference to “a disease,” “a disease state”, or “a nucleic acid” is a reference to one or more such embodiments, and includes equivalents thereof known to those skilled in the art and so forth.

As used herein, the term “about” or “approximately” in the context of a numerical value or range means±10% of the numerical value or range recited or claimed, unless the context requires a more limited range.

As used herein, “effective” when referring to an amount of a therapeutic compound refers to the quantity of the compound that is sufficient to yield a desired therapeutic response without undue adverse side effects (such as toxicity, irritation, or allergic response) commensurate with a reasonable benefit/risk ratio when used in the manner of this disclosure.

By “reference” is meant a standard or control condition.

The terms “subject,” “patient,” “individual,” and the like as used herein are not intended to be limiting and can be generally interchanged. The subject is a mammal, e.g., a human, a primate, a mouse, a rat, a dog, a cat, a horse, as well as livestock or animals grown for food consumption, e.g., cattle, sheep, pigs, chickens, and goats. In some embodiments, the mammal is a human. The term “subject” as used herein includes a subject diagnosed with glioblastoma.

As used herein, a “symptom” associated with a disorder includes any clinical or laboratory manifestation associated with the disorder, and is not limited to what the subject can feel or observe.

As used herein, the term “therapeutically effective amount” refers to an amount of a therapeutic protein which confers a therapeutic effect on the treated subject, at a reasonable benefit/risk ratio applicable to any medical treatment. The therapeutic effect may be objective (i.e., measurable by some test or marker) or subjective (i.e., subject gives an indication of or feels an effect). In particular, the “therapeutically effective amount” refers to an amount of a therapeutic protein or composition effective to treat, ameliorate, or prevent a desired disease or condition, or to exhibit a detectable therapeutic or preventative effect, such as by ameliorating symptoms associated with the disease, preventing or delaying the onset of the disease, and/or also lessening the severity or frequency of symptoms of the disease. A therapeutically effective amount is commonly administered in a dosing regimen that may comprise multiple unit doses. For any particular therapeutic protein, a therapeutically effective amount (and/or an appropriate unit dose within an effective dosing regimen) may vary, for example, depending on route of administration, on combination with other pharmaceutical agents. Also, the specific therapeutically effective amount (and/or unit dose) for any particular patient may depend upon a variety of factors including the disorder being treated and the severity of the disorder: the activity of the specific pharmaceutical agent employed; the specific composition employed; the age, body weight, general health, sex and diet of the patient; the time of administration, route of administration, and/or rate of excretion or metabolism of the specific fusion protein employed; the duration of the treatment; and like factors as is well known in the medical arts.

As used herein, “treating” encompasses, e.g., inhibition, regression, or stasis of the progression of a disorder. Treating also encompasses the amelioration of a symptom or symptoms of the disorder. As used herein, “inhibition” of disease progression or a disease complication in a subject means preventing or reducing the rate, frequency, or risk of disease progression and/or disease complications in the subject. The terms “preventing” and “prevention” refer to the administration of a therapeutic protocol to a clinically asymptomatic individual who is susceptible or predisposed to a particular adverse condition, disorder, or disease, and thus relates to reducing the risk of the occurrence of symptoms and/or their underlying cause.

The transitional term “comprising,” which is synonymous with “including,” “containing,” or “characterized by,” is inclusive or open-ended and does not exclude additional, unrecited elements or method steps. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention.

### Glioblastoma

Glioblastoma multiforme (“glioblastoma”, “GB”, or “GBM”) is the most common and most aggressive malignant primary brain tumor in humans. GB is highly lethal and characterized by extensive necrosis as well as a high rate of angiogenesis. Treatment typically involves resection, chemotherapy, or radiation. Median survival with no treatment is 4.5 months. Glioblastomas typically contain zones of tissue that are hypoxic, which are highly resistant to radiotherapy, and therefore post-treatment recurrence rates are high.

Glioblastoma is associated with a variety of symptoms. Common symptoms of the disease include seizure, nausea and vomiting, headache, memory loss, and hemiparesis, and progressive memory, personality, or neurological deficit due to temporal and frontal lobe involvement. In some cases, the tumor can start producing symptoms quickly, but occasionally the tumor will grow to be quite large before symptoms appear.

The uncurable nature of GB and poor prognosis result from the resistance that almost all GB patients develop to the standard treatment, such as surgical resection followed by temozolomide chemotherapy and adjuvant radiotherapy. From an immunotherapeutic intervention standpoint, GB tumors are unique compared to their non-cranial tumor counterparts due to the inaccessibility of the tumor and the immune-suppressive nature of the tumor microenvironment (TME) or peritumoral environment. The former prevents drugs from reaching their target effectively through barriers, such as the blood-brain barrier (BBB) and the blood cerebrospinal fluid barrier, while the latter enables brain tumors to protect themselves from immune attack through GB-associated brain cells, such as microglia and astrocytes. These factors and others nullify systemic delivery of large molecules, such as antibody-based technologies e.g., anti-PD1/PD1L, or require high doses of systemic cytokine administration to generate an effective anti-tumor dose at the tumor site. Moreover, the TME is very flexible in the treatment response due to its large variety of immunosuppressive cells in the peritumoral region, enabling activation of treatment-specific resistance programs. Additionally, glioma cells also have a low neoantigen burden, preventing a strong influx of tumor antigen (Ag)-specific immune cells at the tumor site, such as cytotoxic T cells (CTLs), which are crucial to evoking an anti-immune reaction with immunotherapy.

In contrast to tumoricidal drugs that act on tumor cells directly, immunotherapeutics can be at or in the vicinity of the tumor as they work indirectly (Binnewies et al., 2018). Transgenes can be functionally delivered and re-administered through AAVs across species and specifically targeting the nervous system (Chen et al., 2023). Recently, a successful AAV therapy approach has been deployed targeting endothelial cells in the glioma vasculature showing reduced T cell hypofunctionality and promotion of CD8^{POS} T cells (Ramachandran et al., 2023). Thus, described herein are compositions and methods for treating glioblastoma that include AAV-mediated delivery to express 4-1BBL mainly in reactive astrocytes at the TME as a therapeutic reservoir to avoid transgene dilution due to tumor cell proliferation. In some embodiments, AAV vector is packaged in an astrocyte-tropic AAV-F capsid and an GFAP promoter is used to drive the transgene, which is highly active in reactive astrocytes associated with the tumor (Beharry et al., 2022; Hanlon et al., 2019; Yao et al., 2022). In some embodiments, vectors as described herein are injected at the tumor site within three consecutive days to avoid immune inhibition of AAV transduction. The methods described herein provide an elevated 4-1BBL expression at the tumor border prior to rIL-12 treatment. This boosted rIL-12 therapy efficacy and prolonged overall survival.

### Methods of Treatment

The methods described herein include methods for the treatment of glioblastoma. As non-limiting examples, the present methods include administering a treatment comprising any of the compositions described herein, including a nucleic acid encoding for 4-1BBL (optionally in an expression vector), to a subject having glioblastoma. The methods can optionally include administering the 4-1BBL in combination with IL-12. In some embodiments, the 4-1BBL is administered alone without IL-12. In some embodiments, the IL-12 is administered as a recombinant polypeptide, optionally formulated for pharmaceutical use. In some embodiments, the IL-12 is administered as a nucleic acid encoding an IL-12 fusion protein comprising IL-12 and Fc. The IL-12 nucleic acid can be administered in a separate vector, or in the same vector as the 4-1BBL.

The subject to be treated with the present methods can be any mammal e.g., a human or non-human mammal (e.g., a veterinary or zoological subject). Without wishing to be bound by theory, the objective of such therapy is, among other things, is to increase IL-12 stimuli to drive CD8^{POS} T cell recruitment from the blood to the brain, as well as differentiation towards a more effector-like CTL state.

Treating glioblastoma includes treating a subject diagnosed with existing glioblastoma, as well as preventing the recurrence of glioblastoma. In some embodiments, the amount of 4-1BBL or 4-1BBL and IL-12 administered to a subject (either in a single dose or over multiple doses) is effective in one or more of inhibiting growth of glioblastoma cells, inhibiting metastasis of glioblastoma cells, killing glioblastoma cells, reducing tumor size, and reducing severity or incidence of symptoms associated with the presence of glioblastoma cells. The degree of one or more of these therapeutic effects may be about or more than about 10%, 20%, 30%, 40%, 50%, 60%, 70%, 80%, 90%, 95%, or more. In some embodiments, therapeutic efficacy is measured by an increased time in disease progression, such as between the appearance of one or more first symptoms, and the appearance of one or more second symptoms, or delay between two or more occurrences of the same symptoms. Delay may be about or more than about days, weeks, months, or years (e.g., 1, 2, 3, 4, 5, 6, 7, or more days; 1, 2, 3, 4, 5, 6, 7, 8, or more weeks; 1, 2, 3, 4, 5, 6, or more months; or 1, 2, 3, 4, 5, or more years). In the case of prevention, the subject may be an individual at risk of developing glioblastoma, such as a subject in remission, having a family history, and/or having some other predisposition. The degree of therapeutic efficacy may be with respect to a starting condition of the subject (e.g., the size of a tumor, rate of growth, rate of metastasis, severity or incidence of one or more symptoms), or with respect to a reference population (e.g., an untreated population, or a population treated with a different agent).

### 4-1BB Ligand (4-1BBL)

4-1BB (also referred to in the art as “CD137”, tumor necrosis factor ligand superfamily member 9 “TNFRSF9”, etc.) is a receptor belonging to the tumor necrosis factor receptor (TNFR) superfamily. 4-1BB is a co-stimulatory molecule generally expressed in activated T lymphocytes and involved in immunity and autoimmune diseases (Kwon et al. PNAS 84:2896, 1987; Kwon et al. PNAS (1989) 86:1963; Son et al. Journal of Immunological Methods (2004) 286(1-2):187-201, each of which is herein incorporated by reference in its entirety). 4-1BB is expressed on the cell surface in monomer (30 kDa) and dimer (55 kDa) forms and likely trimerizes with 4-1BB ligand to signal.

As used herein, the term “4-1BBL” (4-1BB ligand) means a mammalian polypeptide capable of binding to 4-1BB. It is a type II extracellular membrane polypeptide which has a transmembrane site following this domain and has an extracellular (receptor-binding) domain at the C-terminus of the polypeptide. When the 4-1BB ligand binds to 4-1BB, it initiates the transmission of biological signals in cells bearing the receptor. The recent studies show that 4-1BB and 4-1BBL induces activation, differentiation, and proliferation of T cells, and the 4-1BBL is known to act as an activator of dendritic cells.

“4-1BBL” can include full length 4-1BBL nucleotides and proteins; fragments or variants thereof having biological activity can also be used. Soluble polypeptides, including the extracellular domain of 4-1BBL or receptor binding fragments thereof, are also within the scope of the 4-1BB and 4-1BBL polypeptides as long as they have biological activity. Detailed descriptions of the 4-1BB and 4-1BBL polypeptides are provided in U.S. Pat. Nos. 5,674,704, 7,211,259, and in Alderson et al. Eur. J. Immunol. 24:2219-2227, 1994, and Kim A M J, Nemeth M R and Lim S-O, (2022) 4-1BB: A promising target for cancer immunotherapy. Front. Oncol. 12:968360. The contents of which are incorporated herein by reference.

An exemplary human amino acid sequence of 4-1BBL that can be used in the methods and compositions described herein includes GenBank Accession No. NP_003802.1. An exemplary nucleotide sequence encoding human 4-1BBL that can be used in the present methods and compositions can include nucleotide sequences encoding the amino acid sequence of the 4-1BBL, for example the nucleotide sequence corresponding to the CDS (coding sequence) of the sequence described in GenBank Accession No. NM_003811.4.

### IL-12

Interleukin-12 is a heterodimeric cytokine with multiple biological effects on the immune system. It is composed of two subunits, p35 and p40, both of which are required for the secretion of the active form of IL-12, p70. Interleukin-12 acts on dendritic cells (DC), leading to increased maturation and antigen presentation, which can allow for the initiation of a T cell response to tumor specific antigens. It also drives the secretion of IL-12 by DCs, creating a positive feedback mechanism to amplify the response. Once a response is initiated, IL-12 plays a fundamental role in directing the immune system towards a Th1 cytokine profile, inducing CD4⁺ T cells to secrete interferon-gamma (IFN-γ) and leading to a CD8⁺ cytotoxic T cell response.⁴ However, IL-12 is also a strong pro-inflammatory cytokine that leads to the secretion of other cytokines including tumor necrosis factor-alpha (TNF-α) which, combined with IFN-γ, is a prerequisite for the development of CD4+ cytotoxic T lymphocytes (CTL). Furthermore, IL-12 can promote the activation of innate immune cells such as macrophages and eosinophils through its induction of IFN-γ and other cytokines. This activation then leads to IL-12 secretion by these cells and further amplification of both the innate and acquired responses. However, high levels of IL-12, and consequently IFN-γ, have also been associated with induction of antagonistic molecules such as IL-10 and the depletion of signaling molecules downstream of IL-12, such as STAT4.

Recombinant viral vectors can include IL-12 coding nucleotide sequences in expressible forms to secrete IL-12. In some embodiments, the polynucleotide comprises the sequence of both IL-12 subunits, p35 and p40, separated by an RES sequence which permits expression of multiple transgenes from a single transcript. In other embodiments, the polynucleotide directs expression of an IL-12 fusion polypeptide that retains IL-12 activity, e.g., an IL-12-Fc fusion peptide. Detailed descriptions of IL-12 polypeptides are provided in Jia Z, et al., IL12 immune therapy clinical trial review: Novel strategies for avoiding CRS-associated cytokines. Front Immunol. 2022 Sep. 20:13:952231; Strauss J, et al. First-in-Human Phase I Trial of a Tumor-Targeted Cytokine (NHS-IL12) in Subjects with Metastatic Solid Tumors. Clin Cancer Res 1 Jan. 2019:25 (1): 99-109; Gutierrez, E, et al. An optimized IL-12-Fc expands its therapeutic window, achieving strong activity against mouse tumors as tolerable drug doses, Med 4, 326-340, May 12, 2023.

In some embodiments, IL-12 is provided as a recombinant fusion polypeptide directly to the subject. The IL-12 polypeptide can be an IL-12 polypeptide conjugated to Fc. In some embodiments, the IL-12 polypeptide that has at least 85%, 86%, 87%, 88%, 89%, 90%, 91%, 92%, 93%, 94%, 95%, 96%, 97%, 98%, or 99% or more to one or both of the IL-12 subunits p35 and p40, as identified by SEQ ID NOs: 5, 6, 7 or 8 and retains IL-12 activity. IL-12 activity is determined for example by assessing activation of the IL-12 receptor in a cell-based assay.

### Vectors

Nucleic acids encoding an 4-1BBL and/or IL-12 polypeptide or a therapeutically active fragment thereof can be incorporated into a gene construct to be used as a part of a gene therapy protocol. For example, described herein are targeted expression vectors for in vivo delivery and expression of a polynucleotide that encodes a 4-1BB and/or IL-12 polypeptide or active fragment thereof in particular cell types. Expression constructs can include such components as promoters and can be administered in any effective carrier, e.g., any formulation or composition capable of effectively delivering the component gene to cells in vivo. Approaches include insertion of the gene in viral vectors, preferably adeno-associated virus. Viral vectors typically transduce cells directly.

Viral vectors capable of highly efficient transduction may be employed, including any serotypes of rAAV (e.g., AAV1-AAV12, AAV-9, and AAV-F) vectors, recombinant or chimeric AAV vectors, as well as lentivirus or other suitable viral vectors. A typical approach for in vivo introduction of nucleic acid into a cell is by use of a viral vector containing nucleic acid, e.g., a cDNA encoding 4-1BB or 4-1BB and IL-12. Among other things, infection of cells with a viral vector has the advantage that a large proportion of the targeted cells can receive the nucleic acid. Additionally, molecules encoded within the viral vector, e.g., by a cDNA contained in the viral vector, are expressed efficiently in cells that have taken up viral vector nucleic acid.

A viral vector system particularly useful for delivery of nucleic acids is the adeno-associated virus (AAV). Adeno-associated virus is a naturally occurring defective virus that requires another virus, such as an adenovirus or a herpes virus, as a helper virus for efficient replication and a productive life cycle. (For a review see Muzyczka et al., Curr. Topics in Micro and Immunol. 158:97-129 (1992)). AAV vectors efficiently transduce various cell types and can produce long-term expression of transgenes in vivo. Although AAV vector genomes can persist within cells as episomes, vector integration has been observed (see for example Deyle and Russell, Curr Opin Mol Ther. 2009 August; 11(4): 442-447; Asokan et al., Mol Ther. 2012 April; 20(4): 699-708; Flotte et al., Am. J. Respir. Cell. Mol. Biol. 7:349-356 (1992); Samulski et al., J. Virol. 63:3822-3828 (1989); and Mclaughlin et al., J. Virol. 62:1963-1973 (1989)). AAV vectors, such as AAV2, have been extensively used for gene augmentation or replacement and have shown therapeutic efficacy in a range of animal models as well as in the clinic; see, e.g., Mingozzi and High, Nature Reviews Genetics 12, 341-355 (2011); Deyle and Russell, Curr Opin Mol Ther. 2009 August; 11(4): 442-447; Asokan et al., Mol Ther. 2012 April; 20(4): 699-708. AAV vectors containing as little as 300 base pairs of AAV can be packaged and can produce recombinant protein expression. Protocols for producing recombinant retroviruses and for infecting cells in vitro or in vivo with such viruses are known in the art, e.g., can be found in Ausubel, et al., eds., Current Protocols in Molecular Biology, Greene Publishing Associates, (1989), Sections 9.10-9.14, and other standard laboratory manuals. The use of AAV vectors to deliver constructs for expression in the brain has been described, e.g., in Iwata et al., Sci Rep. 2013; 3:1472; Hester et al., Curr Gene Ther. 2009 October; 9(5):428-33; Doll et al., Gene Therapy 1996, 3(5):437-447; and Foley et al., J Control Release. 2014 Dec. 28; 196:71-8.

Adenoviruses include over 50 serotypes (see, e.g., WO 95/27071, which is herein incorporated by reference). Adenoviruses are tractable through the application of techniques of molecular biology and may not require integration into the host cell genome. Recombinant Ad-derived vectors, including vectors that reduce the potential for recombination and generation of wild-type virus, have been constructed (see, e.g., international patent publications WO 95/00655 and WO 95/11984, which are herein incorporated by reference). In some instances, the AAV vector is selected from the group consisting of AAV1, AAV2, AAV3, AAV4, AAV5, AAV6, AAV7, AAV8, AAV9, AAVrh10, AAV11, and AAV12. In some embodiments, the AAV vector is AAV-9, AAV9-PHP.B, AAV-S, or AAV-F, see WO2020198737A1 herein incorporated by reference in its entirety. In some embodiments, the AAV vector is AAV-9 or AAV-F (see Beharry A, et al. The AAV9 Variant Capsid AAV-F Mediates Widespread Transgene Expression in Nonhuman Primate Spinal Cord After Intrathecal Administration. Hum Gene Ther. 2022 January; 33(1-2):61-75; see also Hanlon K S, et al., Selection of an Efficient AAV Vector for Robust CNS Transgene Expression, Molecular Therapy—Methods & Clinical Development, Volume 15, 2019, Pages 320-332). In some instances, a particular AAV serotype vector may be selected based upon the intended use, e.g., based upon the intended route of administration.

A vector as described herein can be a pseudotyped vector. Pseudotyping provides a mechanism for modulating a vector's target cell population. Pseudotyped vectors are those that contain the genome of one vector, e.g., the genome of one AAV serotype, in the capsid of a second vector, e.g., a second AAV serotype. Methods of pseudotyping are well known in the art. For instance, a vector may be pseudotyped with envelope glycoproteins derived from Rhabdovirus vesicular stomatitis virus (VSV) serotypes (Indiana and Chandipura strains), rabies virus (e.g., various Evelyn-Rokitnicki-Abelseth ERA strains and challenge virus standard (CVS)), Lyssavirus Mokola virus, a rabies-related virus, vesicular stomatitis virus (VSV), Mokola virus (MV), lymphocytic choriomeningitis virus (LCMV), rabies virus glycoprotein (RV-G), glycoprotein B type (FuG-B), a variant of FuG-B (FuG-B2) or Moloney murine leukemia virus (MuLV). A virus may be pseudotyped for transduction of one or more groups of cells.

Without limitation, illustrative examples of pseudotyped vectors include recombinant AAV2/1, AAV2/2, AAV2/5, AAV2/6, AAV2/7, AAV2/8, AAV9, AAVrh10, AAV11, and AAV12 serotype vectors. It is known in the art that such vectors may be engineered to include a transgene encoding a human protein or other protein. In particular instances, the present disclosures can include a pseudotyped AAV9 or AAVrh10 viral vector including a nucleic acid as disclosed herein. See Viral Vectors for Gene Therapy: Methods and Protocols, ed. Machida, Humana Press, 2003.

Various methods for application of AAV vector constructs in gene therapy are known in the art, including methods of modification, purification, and preparation for administration to human subjects (see, e.g., Viral Vectors for Gene Therapy: Methods and Protocols, ed. Machida, Humana Press, 2003). In addition, AAV based gene therapy targeted to cells of the CNS has been described (see, e.g., U.S. Pat. Nos. 6,180,613 and 6,503,888). High titer AAV preparations can be produced using techniques known in the art, e.g., as described in U.S. Pat. No. 5,658,776

A vector construct refers to a polynucleotide molecule including all or a portion of a viral genome and a transgene. In some instances, gene transfer can be mediated by a DNA viral vector, such as an adenovirus (Ad) or adeno-associated virus (AAV). Other vectors useful in methods of gene therapy are known in the art. For example, a construct as disclosed herein can include an alphavirus, herpesvirus, retrovirus, lentivirus, or vaccinia virus.

Non-native regulatory sequences, gene control sequences, promoters, non-coding sequences, introns, or coding sequences can be included in a nucleic acid as disclosed herein. The inclusion of nucleic acid tags or signaling sequences, or nucleic acids encoding protein tags or protein signaling sequences, is further contemplated herein. Typically, the coding region is operably linked with one or more regulatory nucleic acid components.

A promoter included in a nucleic acid as disclosed herein can be a tissue- or cell type-specific promoter, a promoter specific to multiple tissues or cell types, an organ-specific promoter, a promoter specific to multiple organs, a systemic or ubiquitous promoter, or a nearly systemic or ubiquitous promoter. A promoter can include any of the above characteristics or other promoter characteristics known in the art. In some embodiments, a polynucleotide encoding 4-1BB or 4-1BB and IL-12 is operably linked to a promoter suitable for expression in glioblastoma cells, such as glial fibrillary acidic protein (GFAP), described in SEQ ID NOs: 9, 10, 11, and/or 12. Other exemplary promoters include, but are not limited to, human Synapsinl (hSyn1), mMeCP2 promoter (MeCP2), NR2E1, GfABC1D, Aldh111, mMBP, MAG, ICAM-2, CLDN5, Tie-2, vWF, FLT1, TRE, c-FOS, eSARE, ubiquitin C, PGK, cytomegalovirus (CMV) CMV early enhancer/chicken β-actin (CAG), and MND.

In clinical settings, the gene delivery systems for the therapeutic gene can be introduced into a subject by any of a number of methods, each of which is known in the art. For instance, a pharmaceutical preparation of the gene delivery system can be introduced systemically, e.g., by intravenous injection, and specific transduction of the protein in the target cells will occur predominantly from specificity of transfection, provided by the gene delivery vehicle, cell-type or tissue-type expression due to the transcriptional regulatory sequences controlling expression of the receptor gene, or a combination thereof. In other embodiments, initial delivery of the recombinant gene is more limited, with introduction into the subject being quite localized. For example, the gene delivery vehicle can be introduced by intrathecal injection, by catheter or by stereotactic injection.

The pharmaceutical preparation of the gene therapy construct can consist essentially of the gene delivery system in an acceptable diluent, or can comprise a slow release matrix in which the gene delivery vehicle is embedded. Alternatively, where the complete gene delivery system can be produced intact from recombinant cells, e.g., retroviral vectors, the pharmaceutical preparation can comprise one or more cells, which produce the gene delivery system.

### Pharmaceutical Compositions and Methods of Administration

Compositions described herein include an AAV vector comprising a sequence encoding 4-1BBL. In some embodiments, the composition includes any of the AAV serotypes disclosed herein. In some embodiments, the 4-1BBL is driven by a promoter. In some embodiments, the promoter is any of the promoters disclosed herein, including any of the GFAP promoters disclosed in SEQ ID NOs: 9-12. In some embodiments, the composition includes an AAV-F vector comprising a GFAP promoter operably linked to a sequence encoding 4-1BBL. In some embodiments, the 4-1BBL is human 4-1BBL. In some embodiments, the 4-1BBL is mouse 4-1BBL.

In some embodiments, the 4-1BBL composition is administered alone without IL-12. In some embodiments, the composition can include 4-1BBL delivered by via an AAV, administered in conjunction with IL-12. The IL-12 can be administered before, in conjunction with, or after the 4-1BBL, and can be administered via a different route from the 4-1BBL. In some embodiments, the IL-12 is administered intratumorally and/or intracranially.

The methods described herein include pharmaceutical compositions comprising or consisting of a 4-1BBL, optionally with IL-12, as an active ingredient, and methods of use thereof. Pharmaceutical compositions typically include a pharmaceutically acceptable carrier. As used herein the language “pharmaceutically acceptable carrier” includes saline, solvents, dispersion media, coatings, isotonic and absorption delaying agents, and the like, compatible with pharmaceutical administration.

Pharmaceutical compositions are typically formulated to be compatible with its intended route of administration. In some embodiments, the pharmaceutical compositions are administered systemically. Examples of routes of administration include parenteral, e.g., intratumoral, intravenous, intradermal, subcutaneous, or intraperitoneal administration.

Methods of formulating suitable pharmaceutical compositions are known in the art, see, e.g., Remington: The Science and Practice of Pharmacy, 21st ed., 2005; and the books in the series Drugs and the Pharmaceutical Sciences: a Series of Textbooks and Monographs (Dekker, NY). For example, solutions or suspensions used for parenteral, intradermal, or subcutaneous application can include the following components: a sterile diluent such as water for injection, saline solution, fixed oils, polyethylene glycols, glycerin, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use can include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, NJ) or phosphate buffered saline (PBS). In all cases, the composition must be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, or sodium chloride in the composition. Prolonged absorption of the injectable compositions can be brought about by including in the composition an agent that delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, the preferred methods of preparation are vacuum drying and freeze-drying, which yield a powder of the active ingredient plus any additional desired ingredient from a previously sterile-filtered solution thereof.

In some embodiments, the therapeutic compounds are prepared with carriers that will protect the therapeutic compounds against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Such formulations can be prepared using standard techniques, or obtained commercially, e.g., from Alza Corporation and Nova Pharmaceuticals, Inc. Liposomal suspensions (including liposomes targeted to selected cells with monoclonal antibodies to cellular antigens) can also be used as pharmaceutically acceptable carriers. Nanoparticles (1 to 1,000 nm) and microparticles (1 to 1,000 μm), e.g., nanospheres and microspheres and nanocapsules and microcapsules, can also be used. These can be prepared according to methods known to those skilled in the art.

The pharmaceutical compositions can be included in a kit, container, pack, or dispenser together with instructions for administration in a method described herein.

### Exemplary Sequences and Constructs

In some embodiments, the sequence of a protein or nucleic acid used in a composition or method described herein is at least 80%, 85%, 90%, 95%, 97%, 98%, or 99% identical to a reference sequence set forth herein. To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In a preferred embodiment, the length of a reference sequence aligned for comparison purposes is at least 80% of the length of the reference sequence, and in some embodiments is at least 90% or 100%. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. For example, the percent identity between two amino acid sequences can be determined using the Needleman and Wunsch ((1970) J. Mol. Biol. 48:444-453) algorithm which has been incorporated into the GAP program in the GCG software package (available on the world wide web at gcg.com), using the default parameters, e.g., a Blossum 62 scoring matrix with a gap penalty of 12, a gap extend penalty of 4, and a frameshift gap penalty of 5.

human 4-1BBL amino acid
>AAA53134.1 4-1BB ligand [Homo sapiens]
SEQ ID NO: 1
MEYASDASLDPEAPWPPAPRARACRVLPWALVAGLLLLLLLAAACAVFLAC
PWAVSGARASPGSAASPRLREGPELSPDDPAGLLDLRQGMFAQLVAQNVLLI
DGPLSWYSDPGLAGVSLTGGLSYKEDTKELVVAKAGVYYVFFQLELRRVVA
GEGSGSVSLALHLQPLRSAAGAAALALTVDLPPASSEARNSAFGFQGRLLHLS
AGQRLGVHLHTEARARHAWQLTQGATVLGLFRVTPEIPAGLPSPRSE
mouse 4-1BBL amino acid
>AAA39435.1 4-1BB ligand [Mus musculus]
SEQ ID NO: 2
MDQHTLDVEDTADARHPAGTSCPSDAALLRDTGLLADAALLSDTVRPTNAA
LPTDAAYPAVNVRDREAAWPPALNFCSRHPKLYGLVALVLLLLIAACVPIFTR
TEPRPALTITTSPNLGTRENNADQVTPVSHIGCPNTTQQGSPVFAKLLAKNQA
SLCNTTLNWHSQDGAGSSYLSQGLRYEEDKKELVVDSPGLYYVFLELKLSPT
FTNTGHKVQGWVSLVLQAKPQVDDFDNLALTVELFPCSMENKLVDRSWSQL
LLLKAGHRLSVGLRAYLHGAQDAYRDWELSYPNTTSFGLFLVKPDNPWE
human 4-1BBL mRNA, complete cds
>U03398.1 Human receptor 4-1BB ligand mRNA, complete cds
SEQ ID NO: 3
GTCATGGAATACGCCTCTGACGCTTCACTGGACCCCGAAGCCCCGTGGCC
TCCCGCGCCCCGCGCTCGCGCCTGCCGCGTACTGCCTTGGGCCCTGGTCGC
GGGGCTGCTGCTGCTGCTGCTGCTCGCTGCCGCCTGCGCCGTCTTCCTCGC
CTGCCCCTGGGCCGTGTCCGGGGCTCGCGCCTCGCCCGGCTCCGCGGCCA
GCCCGAGACTCCGCGAGGGTCCCGAGCTTTCGCCCGACGATCCCGCCGGC
CTCTTGGACCTGCGGCAGGGCATGTTTGCGCAGCTGGTGGCCCAAAATGT
TCTGCTGATCGATGGGCCCCTGAGCTGGTACAGTGACCCAGGCCTGGCAG
GCGTGTCCCTGACGGGGGGCCTGAGCTACAAAGAGGACACGAAGGAGCT
GGTGGTGGCCAAGGCTGGAGTCTACTATGTCTTCTTTCAACTAGAGCTGCG
GCGCGTGGTGGCCGGCGAGGGCTCAGGCTCCGTTTCACTTGCGCTGCACC
TGCAGCCACTGCGCTCTGCTGCTGGGGCCGCCGCCCTGGCTTTGACCGTGG
ACCTGCCACCCGCCTCCTCCGAGGCTCGGAACTCGGCCTTCGGTTTCCAGG
GCCGCTTGCTGCACCTGAGTGCCGGCCAGCGCCTGGGCGTCCATCTTCAC
ACTGAGGCCAGGGCACGCCATGCCTGGCAGCTTACCCAGGGCGCCACAGT
CTTGGGACTCTTCCGGGTGACCCCCGAAATCCCAGCCGGACTCCCTTCACC
GAGGTCGGAATAACGCCCAGCCTGGGTGCAGCCCACCTGGACAGAGTCCG
AATCCTACTCCATCCTTCATGGAGACCCCTGGTGCTGGGTCCCTGCTGCTT
TCTCTACCTCAAGGGGCTTGGCAGGGGTCCCTGCTGCTGACCTCCCCTTGA
GGACCCTCCTCACCCACTCCTTCCCCAAGTTGGACCTTGATATTTATTCTG
AGCCTGAGCTCAGATAATATATTATATATATTATATATATATATATATTTC
TATTTAAAGAGGATCCTGAGTTTGTGAATGGACTTTTTTAGAGGAGTTGTT
TTGGGGGGGGGGTCTTCGACATTGCCGAGGCTGGTCTTGAACTCCTGGAC
TTAGACGATCCTCCTGCCTCAGCCTCCCAAGCAACTGGGATTCATCCTTTC
TATTAATTCATTGTACTTATTTGCCTATTTGTGTGTATTGAGCATCTGTAAT
GTGCCAGCATTGTGCCCAGGCTAGGGGGCTATAGAAACATCTAGAAATAG
ACTGAAAGAAAATCTGAGTTATGGTAATACGTGAGGAATTTAAAGACTCA
TCCCCAGCCTCCACCTCCTGTGTGATACTTGGGGGCTAGCTTTTTTCTTTCT
TTCTTTTTTTTGAGATGGTCTTGTTCTGTCAACCAGGCTAGAATGCAGCGG
TGCAATCATGAGTCAATGCAGCCTCCAGCCTCGACCTCCCGAGGCTCAGG
TGATCCTCCCATCTCAGCCTCTCGAGTAGCTGGGACCACAGTTGTGTGCCA
CCACACTTGGCTAACTTTTTAATTTTTTTGCGGAGACGGTATTGCTATGTTG
CCAAGGTTGTTTACATGCCAGTACAATTTATAATAAACACTCATTTTTCC
mouse 4-1BBL mRNA, complete cds
>L15435.1 Mus musculus 4-1BB ligand mRNA, complete cds
SEQ ID NO: 4
AGCCTATAAAGCACGGGCACTGGCGGGAGACGTGCACTGACCGACCGTG
GTAATGGACCAGCACACACTTGATGTGGAGGATACCGCGGATGCCAGACA
TCCAGCAGGTACTTCGTGCCCCTCGGATGCGGCGCTCCTCAGAGATACCG
GGCTCCTCGCGGACGCTGCGCTCCTCTCAGATACTGTGCGCCCCACAAAT
GCCGCGCTCCCCACGGATGCTGCCTACCCTGCGGTTAATGTTCGGGATCGC
GAGGCCGCGTGGCCGCCTGCACTGAACTTCTGTTCCCGCCACCCAAAGCT
CTATGGCCTAGTCGCTTTGGTTTTGCTGCTTCTGATCGCCGCCTGTGTTCCT
ATCTTCACCCGCACCGAGCCTCGGCCAGCGCTCACAATCACCACCTCGCC
CAACCTGGGTACCCGAGAGAATAATGCAGACCAGGTCACCCCTGTTTCCC
ACATTGGCTGCCCCAACACTACACAACAGGGCTCTCCTGTGTTCGCCAAG
CTACTGGCTAAAAACCAAGCATCGTTGTGCAATACAACTCTGAACTGGCA
CAGCCAAGATGGAGCTGGGAGCTCATACCTATCTCAAGGTCTGAGGTACG
AAGAAGACAAAAAGGAGTTGGTGGTAGACAGTCCCGGGCTCTACTACGT
ATTTTTGGAACTGAAGCTCAGTCCAACATTCACAAACACAGGCCACAAGG
TGCAGGGCTGGGTCTCTCTTGTTTTGCAAGCAAAGCCTCAGGTAGATGACT
TTGACAACTTGGCCCTGACAGTGGAACTGTTCCCTTGCTCCATGGAGAAC
AAGTTAGTGGACCGTTCCTGGAGTCAACTGTTGCTCCTGAAGGCTGGCCA
CCGCCTCAGTGTGGGTCTGAGGGCTTATCTGCATGGAGCCCAGGATGCAT
ACAGAGACTGGGAGCTGTCTTATCCCAACACCACCAGCTTTGGACTCTTTC
TTGTGAAACCCGACAACCCATGGGAATGAGAACTATCCTTCTTGTGACTC
CTAGTTGCTAAGTCCTCAAGCTGCTATGTTTTATGGGGTCTGAGCAGGGGT
CCCTTCCATGACTTTCTCTTGTCTTTAACTGGACTTGGTATTTATTCTGAGC
ATAGCTCAGACAAGACTTTATATAATTCACTAGATAGCATTAGTAAACTG
CTGGGCAGCTGCTAGATAAAAAAAAATTTCTAAATCAAAGTTTATATTTAT
ATTAATATATAAAAATAAATGTGTTTGTAAAT
human IL-12, p35 subunit, amino acid
SEQ ID NO: 5
RNLPVATPDPGMFPCLHHSQNLLRAVSNMLQKARQTLEFYPCTSEEIDHEDIT
KDKTSTVEACLPLELTKNESCLNSRETSFITNGSCLASRKTSFMMALCLSSIYE
DLKMYQVEFKTMNAKLLMDPKRQIFLDQNMLAVIDELMQALNFNSETVPQK
SSLEEPDFYKTKIKLCILLHAFRIRAVTIDRVMSYLNAS
human IL-12, p40 subunit, amino acid
SEQ ID NO: 6
IWELKKDVYVVELDWYPDAPGEMVVLTCDTPEEDGITWTLDQSSEVLGSGK
TLTIQVKEFGDAGQYTCHKGGEVLSHSLLLLHKKEDGIWSTDILKDQKEPKN
KTFLRCEAKNYSGRFTCWWLTTISTDLTFSVKSSRGSSDPQGVTCGAATLSAE
RVRGDNKEYEYSVECQEDSACPAAEESLPIEVMVDAVHKLKYENYTSSFFIR
DIIKPDPPKNLQLKPLKNSRQVEVSWEYPDTWSTPHSYFSLTFCVQVQGKSK
REKKDRVFTDKTSATVICRKNASISVRAQDRYYSSSWSEWASVPCS
human IL-12, p35, subunit, amino acid
>sp|P29459.2|IL12A_HUMAN RecName: Full = Interleukin-12
subunit alpha; Short = IL-12A; AltName: Full = Cytotoxic
lymphocyte maturation factor 35 kDa subunit; Short =
CLMF p35; AltName: Full = IL-12 subunit p35; AltName:
Full = NK cell stimulatory factor chain 1; Short = NKSF1;
Flags: Precursor
SEQ ID NO: 7
MCPARSLLLVATLVLLDHLSLARNLPVATPDPGMFPCLHHSQNLLRAVSNML
QKARQTLEFYPCTSEEIDHEDITKDKTSTVEACLPLELTKNESCLNSRETSFITN
GSCLASRKTSFMMALCLSSIYEDLKMYQVEFKTMNAKLLMDPKRQIFLDQN
MLAVIDELMQALNFNSETVPQKSSLEEPDFYKTKIKLCILLHAFRIRAVTIDRV
MSYLNAS
human IL-12, p40 subunit, amino acid
>sp|P29460.1|IL12B_HUMAN RecName: Full = Interleukin-12
subunit beta; Short = IL-12B; AltName: Full-Cytotoxic
lymphocyte maturation factor 40 kDa subunit; Short =
CLMF p40; AltName: Full = IL-12 subunit p40; AltName:
Full = NK cell stimulatory factor chain 2; Short = NKSF2;
Flags: Precursor
SEQ ID NO: 8
MCHQQLVISWFSLVFLASPLVAIWELKKDVYVVELDWYPDAPGEMVVLTCD
TPEEDGITWTLDQSSEVLGSGKTLTIQVKEFGDAGQYTCHKGGEVLSHSLLLL
HKKEDGIWSTDILKDQKEPKNKTFLRCEAKNYSGRFTCWWLTTISTDLTFSV
KSSRGSSDPQGVTCGAATLSAERVRGDNKEYEYSVECQEDSACPAAEESLPIE
VMVDAVHKLKYENYTSSFFIRDIIKPDPPKNLQLKPLKNSRQVEVSWEYPDT
WSTPHSYFSLTFCVQVQGKSKREKKDRVFTDKTSATVICRKNASISVRAQDR
YYSSSWSEWASVPCS
glial fibrillary acidic protein, isoform 1
>NP_002046.1 glial fibrillary acidic protein isoform 1
[Homo sapiens]
SEQ ID NO: 9
MERRRITSAARRSYVSSGEMMVGGLAPGRRLGPGTRLSLARMPPPLPTRVDF
SLAGALNAGFKETRASERAEMMELNDRFASYIEKVRFLEQQNKALAAELNQ
LRAKEPTKLADVYQAELRELRLRLDQLTANSARLEVERDNLAQDLATVRQK
LQDETNLRLEAENNLAAYRQEADEATLARLDLERKIESLEEEIRFLRKIHEEEV
RELQEQLARQQVHVELDVAKPDLTAALKEIRTQYEAMASSNMHEAEEWYRS
KFADLTDAAARNAELLRQAKHEANDYRRQLQSLTCDLESLRGTNESLERQM
REQEERHVREAASYQEALARLEEEGQSLKDEMARHLQEYQDLLNVKLALDIE
IATYRKLLEGEENRITIPVQTFSNLQIRETSLDTKSVSEGHLKRNIVVKTVEMR
DGEVIKESKQEHKDVM
glial fibrillary acidic protein, isoform 2
>NP_001124491.1 glial fibrillary acidic protein isoform 2
[Homo sapiens]
SEQ ID NO: 10
MERRRITSAARRSYVSSGEMMVGGLAPGRRLGPGTRLSLARMPPPLPTRVDF
SLAGALNAGFKETRASERAEMMELNDRFASYIEKVRFLEQQNKALAAELNQ
LRAKEPTKLADVYQAELRELRLRLDQLTANSARLEVERDNLAQDLATVRQK
LQDETNLRLEAENNLAAYRQEADEATLARLDLERKIESLEEEIRFLRKIHEEEV
RELQEQLARQQVHVELDVAKPDLTAALKEIRTQYEAMASSNMHEAEEWYRS
KFADLTDAAARNAELLRQAKHEANDYRRQLQSLTCDLESLRGTNESLERQM
REQEERHVREAASYQEALARLEEEGQSLKDEMARHLQEYQDLLNVKLALDIE
IATYRKLLEGEENRITIPVQTFSNLQIRGGKSTKDGENHKVTRYLKSLTIRVIPI
QAHQIVNGTPPARG
glial fibrillary acidic protein, isoform 3
>NP_001229305.1 glial fibrillary acidic protein isoform 3
[Homo sapiens]
SEQ ID NO: 11
MERRRITSAARRSYVSSGEMMVGGLAPGRRLGPGTRLSLARMPPPLPTRVDF
SLAGALNAGFKETRASERAEMMELNDRFASYIEKVRFLEQQNKALAAELNQ
LRAKEPTKLADVYQAELRELRLRLDQLTANSARLEVERDNLAQDLATVRQK
LQDETNLRLEAENNLAAYRQEADEATLARLDLERKIESLEEEIRFLRKIHEEEV
RELQEQLARQQVHVELDVAKPDLTAALKEIRTQYEAMASSNMHEAEEWYRS
KFADLTDAAARNAELLRQAKHEANDYRRQLQSLTCDLESLRGTNESLERQM
REQEERHVREAASYQEALARLEEEGQSLKDEMARHLQEYQDLLNVKLALDIE
IATYRKLLEGEENRITIPVQTFSNLQIRGQYSRASWEGHWSPAPSSRACRLLQT
GTEDQGKGIQLSLGAFVTLQRS
glial fibrillary acidic protein, isoform 4
>NP_001350775.1 glial fibrillary acidic protein isoform 4
[Homo sapiens]
SEQ ID NO: 12
MERRRITSAARRSYVSSGEMMVGGLAPGRRLGPGTRLSLARMPPPLPTRVDF
SLAGALNAGFKETRASERAEMMELNDRFASYIEKVRFLEQQNKALAAELNQ
LRAKEPTKLADVYQAELRELRLRLDQLTANSARLEVERDNLAQDLATVRQK
LQDETNLRLEAENNLAAYRQEADEATLARLDLERKIESLEEEIRFLRKIHEEEV
RELQEQLARQQVHVELDVAKPDLTAALKEIRTQYEAMASSNMHEAEEWYRS
KFADLTDAAARNAELLRQAKHEANDYRRQLQSLTCDLESLRGTNESLERQM
REQEERHVREAASYQEALARLEEEGQSLKDEMARHLQEYQDLLNVKLALDIE
IATYRKLLEGEENRITIPVQTFSNLQIRGGKSTKDGENHKVTRYLKSLTIRVIPI
QAHQIVNGTPPARETSLDTKSVSEGHLKRNIVVKTVEMRDGEVIKESKQEHK
DVM

### EXAMPLES

The subject matter is further described in the following examples, which do not limit claim scope.

### Example 1: Method Details

Intracranial Tumor Implantation. CD8-Depletion and Retroorbital Blood Collection

Adult mice were anesthetized using 2.5% isoflurane (USP, Baxter Healthcare cooperation) in 100% oxygen via a nose cone and placed on a warm pad to avoid hypothermia. A total of 5×10⁴ CT-2A-Fluc were suspended in 1 uL Opti-MEM (Gibco, Waltham, MA). In total 2 μL of cell suspension was then implanted into the left striatum of C57BL/6J mice, 40 IL-12 p40-YFP or 40 IL-12p40 KO (Il12^{-/-}) mice using a Hamilton syringe (Sigma-Aldrich, Germany) and automatic stereotaxic injector (Stoelting, Wood Dale, IL) with a flow rate of 0.2 μl/min for 10 min. In reference to bregma, three coordinates for stereotactic implantation were chosen: anterior-posterior (AP)=2.0 mm, medial-lateral=0.5 mm, and dorsal-ventral=2.5 mm. Overall survival of the mice was based on 20% weight loss, presence of apparent distress, or actual death. Tumor growth in mice was assessed by measuring bioluminescence using IVIS100 (PerkinElmer, Waltham, MA) every three or four days starting from day 7 after tumor implantation. For CT-2A tumors, ten days after intracranial injection, mice were treated with either (5, 20, 50, 200, or 500 ng) rIL-12-FC or FC (50 ng) sham control by intracranial injections using a Hamilton syringe (Sigma-Aldrich, Germany) and automatic stereotaxic injector (Stoelting, Wood Dale, IL) with a flow rate of 0.2 μl/min for 10 min at the coordinates used for tumor implantations.

To deplete CD8 T cells in C57BL/6J and Il12^{-/-} mice, endogenous CD8 T cells were depleted by i.v. injection of anti-CD8 antibody or IgG control (Lyt 3.2) (Bioxcell) at day 9 (50 μg) and day 10 (100 μg) post-tumor implantation. At day 10, rIL-12 or sham was injected i.e. at the tumor site and mice were sacrificed at day 18 for flow cytometry of dissociated brain cells.

To identify the depletion of CD8 T cells in the blood post-i.v. injection of anti-CD8 antibody, 100 μl retro-orbital blood was collected via capillary in EDTA tubes to avoid coagulation on days 7, 11 and 18 post-tumor implantation. The collected blood was further processed immediately for RNA isolation.

### AAV Plasmid Constructs and Production

The m4-1BBL expression construct was cloned into a GFAP-GFP AAVF vector plasmid (AltaBiotech), using the restriction enzymes NheI-HF and NcoI-HF (New England Biolabs) followed by Gibson assembly with NEBuilder® HiFi DNA Assembly Master Mix (New England Biolabs). Both the AAVF-m41BBL and AAVF-null vector plasmids were then transformed into SURE Electroporation Competent cells (Agilent Technologies) by two pulses at 1700 V. Plasmid DNA was isolated in nuclease-free water (Ambion Life Technologies) using the Qiaprep® Spin miniprep kit (Qiagen) after selection with 1 μg/mL ampicillin (ampicillin sodium salt, Sigma). Both plasmid constructs were fully sequenced with Next Generation Sequencing at the MGH CCIB DNA core and analyzed with Snapgene software version 6.0.2. Upon confirmation of the sequence, the plasmid constructs were isolated at a large scale by AltaBiotech at a concentration of 2 μg/μL. Subsequently, scAAVF vectors were produced by Packgene at a titer of 1.0×10\hat{ }13 genome copies (gc)/mL.

### Western Blots

Total protein was extracted from cultured cells using RIPA lysis buffer (Thermo Scientific). The tissue samples were homoginized in RIPA lysis buffer with a tissue homogenizer. RIPA buffer was supplemented with a protease inhibitor cocktail (Sigma-Aldrich). To remove non-soluble cell debris, samples were sonicated using a probe sonicator (Sonic Dismembrator Model 100, Fisher Scientific) at a setting of 3.0 for 5 sec and centrifuged at 15,000×g for 10 min at 4° C. Protein concentration was determined using the Pierce™ BCA Protein Assay Kit (Thermo Fisher Scientific). Absorbance was measured at 562 nm using the SynergyHI microplate reader (BioTek). Equal amounts of protein (20 μg) mixed with Laemmli SDS-Sample buffer (Boston BioProducts) were loaded and resolved by electrophoresis on NuPage® 4-12% Bis-Tris polyacrylamide gels (Thermo Fisher Scientific) in NuPage® MES SDS Running Buffer (Thermo Fisher Scientific). After transfer onto nitrocellulose membranes using the iBlot 2 (Thermo Fisher Scientific), samples were subsequently incubated for one hour at RT in 5% non-fat dry milk (Labscientific) in Tris-buffered saline (pH=7.4) with 0.05% Tween 20 (TBS-T) and probed with primary antibody mouse 3×FLAG-tag 1:1000 (Merck, F3165) or goat-α-β-actin (Santa Cruz Biotechnology, I-19) overnight at 4° C. After washing three times with TBS-T for 10 min, membranes were incubated for 1 hr at RT with secondary antibodies ECL™ donkey-anti-goat immunoglobulin G (IgG) (Sigma-Aldrich) and ECL™ sheep-anti-mouse IgG (Thermo Fisher Scientific) (1:5000) corresponding to the primary antibodies. Membranes were developed with ECL or Femto staining (Thermo Fisher Scientific) and imaged on an Azure Biosystems C300 gel imager.

### RT-qPCR

Total RNA was extracted using the Direct-Zol RNAmini kit (Zymo-research). RNA concentrations were measured using the Nanodrop Spectrophotometer ND-1000 (Thermo Fisher Scientific). For gene expression analysis using RT-qPCR, cDNA was synthesized from 200 ng total RNA and prepared using the SuperScript® Vilo™ cDNA Synthesis Kit (Thermo Fisher Scientific). cDNA samples were diluted 10-fold with nuclease-free water. Gene expression was determined using the manufacturing protocol of PowerUp™ SYBR™ Green PCR Master Mix (Applied Biosystems). The cycling conditions using the standard protocol were: 2 min at 50° C., 10 min at 95° C., 40 cycles of 95° C. for 15 sec and 60° C. for 1 min, followed by a melt curve from 60 to 95° C. at 0.1° C./sec, with 15 sec hold at 95° C. Twenty-five sets of primers (Supplementary Information Table 1) obtained from Origene (origene.com/) were used to specifically target the genes of interest by RT-qPCR. Gene expression was normalized to the housekeeping mRNA β-Actin.

### Whole Blood Collection and Tissue Digestion

Mice were sacrificed by lethal intraperitoneal injection of 100 μL containing ketamine (5 μL) and xylazine (45 μL) and saline (50 μL) (Patterson Veterinary). Upon ceasing of all reflexes, whole blood was collected directly from the heart in EDTA tubes. The blood was processed immediately by centrifugation at 1500×g for 15 min to pellet the blood cells. The supernatant was carefully centrifuged again at 2500×g to collect the plasma. The plasma was further analyzed at the Pathology core at MGH, with the comprehensive blood toxicology panel.

The mice were exsanguinated further with PBS perfusion. Tumor Tissue Dissociation Kit (MiltenyiBiotec) was used to process the brain into a single-cell suspension. Brains were placed into a GentleMacs C-tube (Miltenyi Biotec) with 2.35 mL RPMI 1640 containing enzymes D (100 μl), R (30 μl) and A (3.5 μl). According to the manufacturer's protocol, the brains were dissociated using the gentle MACS Dissociator (Miltenyi Biotec) on the brain program settings. Samples were run through a 70 μm filter to obtain a single-cell suspension. Myelin removal was achieved using magnetic separation and anti-myelin beads (Miltenyi Biotec). The final cell suspension was resuspended in 1× Dulbecco's (D) PBS without calcium (Ca²⁺) or magnesium (Mg²⁺) (Corning), supplemented with 2 mM EDTA (Thermo Fisher) and 0.5% BSA (Sigma). Samples were then loaded onto a series of LS columns containing microbeads conjugated to anti-mouse CD11b and anti-mouse CD45 (Miltenyi Biotec), respectively, and separated into CD11b^{POS}, CD11b^{NEG}CD45^{POS}, and CD11b^{NEG}CD45^{NEG} (non-immune) cell populations using the MACS multi-stand (Milteny Biotec).

### Antibody Staining and Flow Cytometry

Cell surface proteins were stained for 20 min at 4° C. Intracellular and nuclear proteins were stained for 60 min at RT after permeabilization and fixation (Thermo Fisher Scientific) for 30 min at RT. To investigate T cells, samples were stained with different antibodies. Stained cell samples were re-suspended in 200 μL FACS buffer (Dulbecco's PBS supplemented with 2 mM EDTA and 0.5% FBS) and transferred to FACS tubes (Stellar Scientific). A mixture of isolated lymph nodes derived from the thigh and spleens, were passed through 40 mm cell strainers with red blood cell lysis buffer (Boston Bioproducts) and used as single-stained controls. In all experiments, lymph nodes, spleens and ipsilateral hemispheres implanted with CT-2A cells were mixed together to measure the Fluorescence Minus One (FMOs). For all studies, dead cells were stained using the fixable viability violet dyes-Zombie Red or Zombie Blue (Invitrogen) for 10 min at room temperature, followed by blocking of Fc receptors with TruStain fcX (Biolegend) for 15 min at 4° C. Cells were analyzed on LSRFortessa™ or LSRFortessa X-20 flow cytometers (BD Biosciences) and data was analyzed with FlowJosoftware version 10.8.1.

### Immunohistochemistry

Brain slices of 12 μm were sectioned onto microscope slides (Fisherbrand, Canada) and fixed with 4% paraformaldehyde for 10 min at RT. After fixation slices were rinsed with PBS for 5 min and blocked in blocking buffer (using 5% goat serum and 0.1% Tween-20 in PBS (PBS-T) for 1 hour at RT. Brain slices were then incubated with the primary antibodies (GFP 1:400, Invitrogen Cat #A11120; GFAP 1:400, Invitrogen Cat #13-0300; CD8 1:400, Novus Biologicals Cat #NBP2-29475; IL12Rb1 1:400, Invitrogen Cat #PA5-95976; anti-4-1BB 1:100, Absolute Antibody Cat #Ab01052; 3×FLAG-tag 1:400, Abcam Cat #ab245893: 4-1BBL 1:100, Invitrogen Cat #MA529838; CD11c 1:400, Abcam Cat #ab33483), diluted in blocking buffer at 4° C. overnight. Slices were rinsed three times in PBS-T for 5 min each. Secondary antibodies (goat anti-rabbit 1:400 Invitrogen Cat #A11008; goat anti-rat 1:400 Abcam Cat #ab150157; 1:400 goat anti-mouse Invitrogen Cat #A11001) were diluted in PBS-T and incubated for 1 hr in the dark at RT. Slices were mounted with DAPI (Vectashield, Vector Labs, San Francisco, CA).

For detection of cells undergoing apoptosis the One-step TUNEL In Situ Apoptosis Kit (Green, FITC) (Elabscience) was used following manufacturer's protocol. Fluorescence microscopy images were acquired on the Keyence microscope and were processed using ImageJ 1.49v software.

### Hematoxylin & Eosin Staining

For H&E staining, brain slices were air dried under a fan for 20 min, before fixation in 100% ethanol for 10 min. Brains were rinsed briefly in MilliQ (EMD Millipore), then stained for 10 min at RT with Harris Hematoxylin (Poly Scientific R&D). Slides were washed twice with MilliQ for 2 min, then de-stained in 1% acetic acid (Sigma-Aldrich) for 6 sec, followed by washing twice in MilliQ. Samples were differentiated in 0.05% aqueous lithium carbonate (Poly Scientific R&D) for 30 sec, after which they were washed in warm tap water for 2 min. 1% Eosin Y solution (Electron Microscopy Sciences) was pipetted on top of the sections to counterstain for 4 sec. Next, brains were de-stained in 95% ethanol for 20 sec, followed by further de-staining and dehydration in 100% ethanol for 5 min. Brain sections were cleared in Xylene (Sigma-Aldrich) for 15 min, mounted with Permount (Electron Miscroscopy Sciences) and imaged on a Keyence microscope at 4× magnification.

### Single Cell RNA Sequencing Analysis

Gene-cell count and cell annotation matrices were obtained via the Brain Immune Atlas (brainimmuneatlas.org), containing GL261 tumor-bearing mice, newly diagnosed (primary) GB patients, and patients with recurrent GB samples (Pombo Antunes et al., 2021). The Seurat v4 R package was used to preprocess and analyze the data (Hao et al., 2021). Unless otherwise stated, the Seurat workflow was followed, and quantitative parameters were set to default values. Low-quality cells were excluded from the analysis. The gene expression values were normalized using global-scaling normalization (“LogNormalize” method). The “FindVariableFeature” function was used to detect the top2000 most variable genes using variance stabilizing transformation (“vst”). The expression values for each gene across all cells were scaled using the “scaleData” function. Dimensionality reduction was performed using principal component analysis (PCA). For clustering, “FindNeighbors” and “FindClusters” functions were utilized. Clustering results were visualized using the Uniform Manifold Approximation and Projection (UMAP) Cell types were defined using the original cell annotation matrices (Pombo Antunes et al., 2021). To examine the expression levels of genes of interest, “VlnPlot”, “FeaturePlot” and “AverageExpression” functions from Seurat were used. The proportion of cells per cluster that expressed genes of interest (normalized counts >0) were also calculated.

### Quantification and Statistical Analysis

Bar graphs, heatmaps, and survival plots were made in GraphPad Prism 9.5.1. Error bars show the mean±standard error of the mean (SEM). A one-way ANOVA, two-way ANOVA, multiple t-tests and log rank test were applied to determine if conditions significantly differed. Statistical significance was specified as p<0.05. Sequences and plasmid constructs were analyzed with Snapgene software version 6.0.2.

### Example 2: Intratumoral rIL-12 Prolongs Survival of GB-Bearing Mice

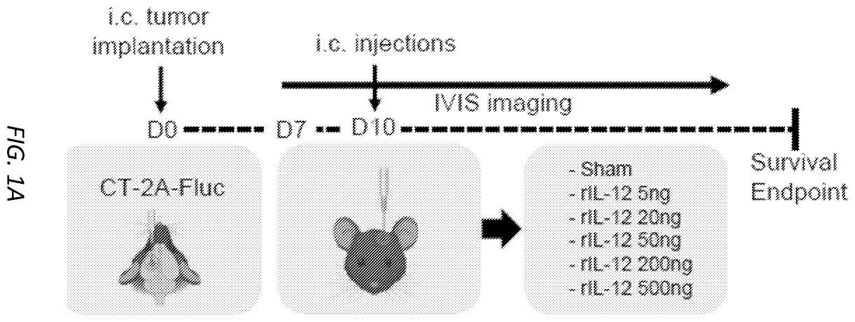

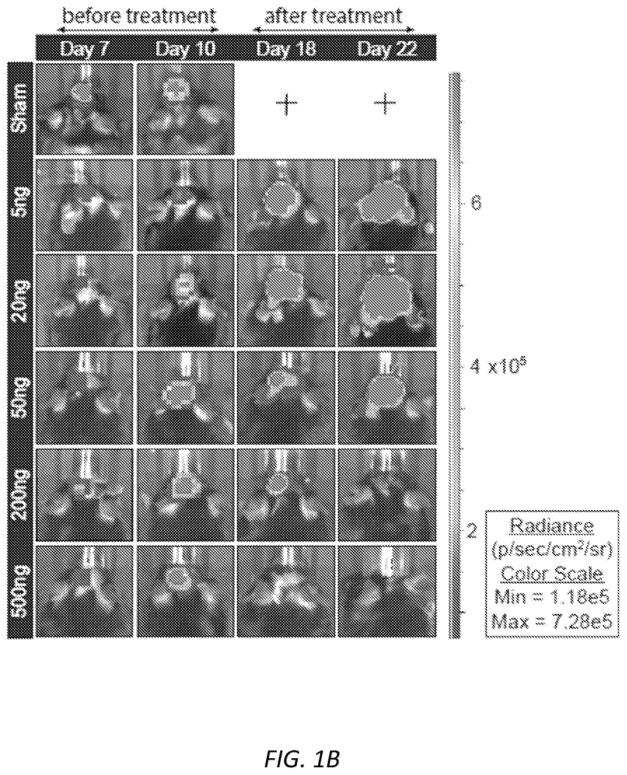

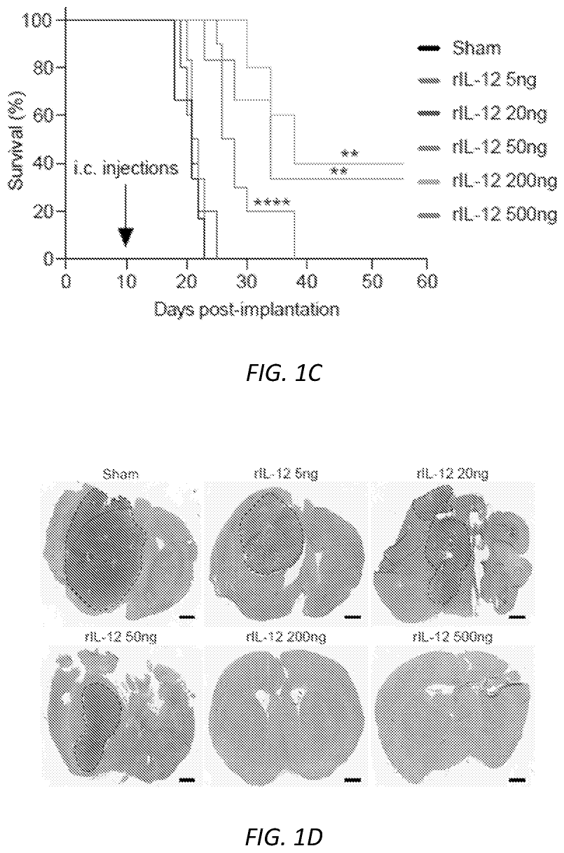

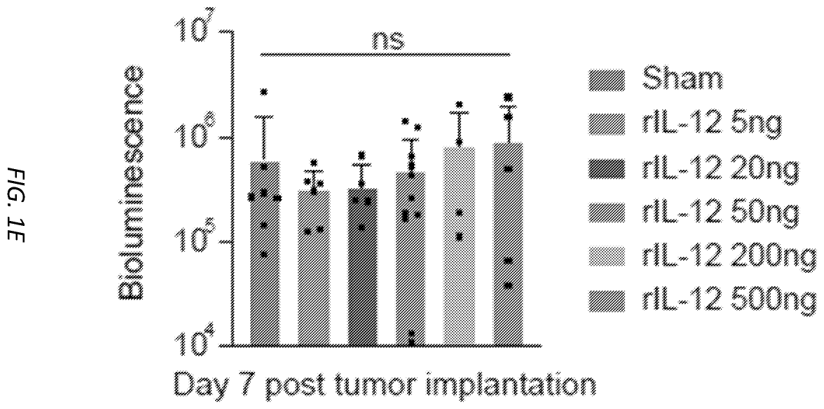

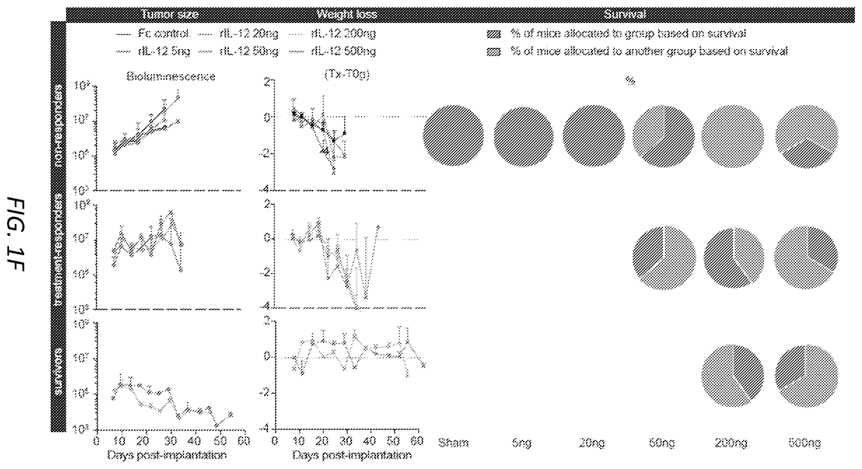

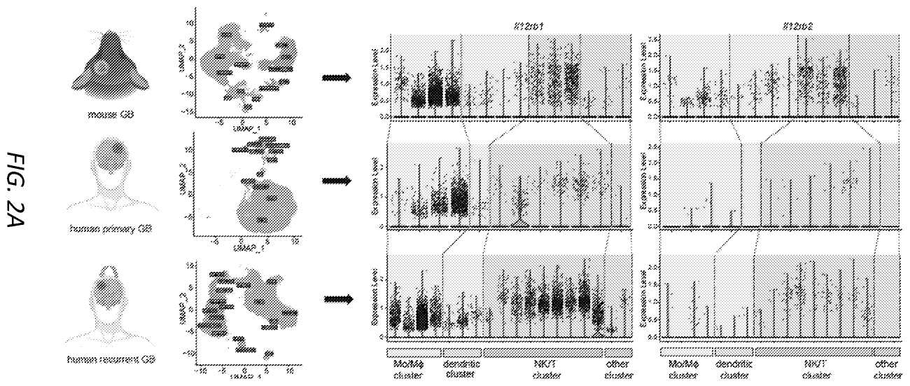

IL-12 is a heterodimeric cytokine composed of p35 (IL-12a) and p40 (IL-12b) subunits, together forming the bioactive IL-12p70 complex (Watford et al., 2003). Proinflammatory cytokines, such as IL-12, are able to reduce the immunosuppressive nature of the TME (Rossari et al., 2023). To evaluate the efficacy of IL-12 in enhancing immunity against GB, we initially utilized a CT-2A glioma cell line, considered a clinically relevant glioma syngeneic mouse model (Liu et al., 2020). Ten days after tumor engraftment we administered murine recombinant IL-12 conjugated to Fc (referred to as rIL-12) directly into the tumor site (FIG. 1A). The intracranially (i.e.) implanted GB cells expressed firefly luciferase (Fluc), allowing us to estimate tumor (CT-2A-Fluc) size in vivo based on the bioluminescence signal post-intraperitoneal injection with the substrate luciferin. At day 7 post-tumor cell implantation all mice displayed similar Fluc levels (FIG. 1E). Subsequently, various concentrations of rIL-12, ranging from 5 ng to 500 ng per mouse, were injected into the tumor and compared to sham control (Fc without the rIL-12 fusion). This approach aided us in monitoring disease progression through assessment of Fluc bioluminescence and body weight changes (FIGS. 1B and 1F). Based on these parameters, we could classify the treated GB-bearing mice into three distinct response patterns: non-responders, treatment-responders, and treatment-survivors (FIG. 1F). Within the non-survivors, mice exhibited similar outcomes to the sham control, characterized by a steady increase in tumor size and a decline in body weight, indicating poor health. This response pattern included all the GB-bearing mice treated with 5 ng and 20 ng rIL-12. Notably, a cohort of the mice, specifically 64% and 33% of the GB mice treated with 50 ng and 500 ng rIL-12, respectively, exhibited a similar response to the sham-treated GB-bearing mice. The treatment-responders showed delayed outgrowth of the tumor cells due to rIL-12 treatment. When compared to the non-responders, a slower increase in bioluminescent signal was observed as well as minimal decrease in body mass. This group contained 36%, 60% and 33% of the 50 ng, 200 ng and 500 ng rIL-12-treated GB-bearing mice, respectively. The treatment-survivors demonstrated favorable outcomes with rIL-12 treatment; 40% and 33% of GB-bearing mice treated with 200 ng and 500 ng rIL-12, respectively, displayed tumor regression concomitant with stable body weight (FIG. 1F). The mice in this response pattern lived at least 60 days with no apparent health concerns.

The varying response patterns of mice to different doses of rIL-12 resulted in discrepancies in terms of overall survival (FIG. 1C). Mice treated with either 50 ng, 200 ng and 500 ng had a significant benefit compared to the sham group, which had a median survival of 21 days post-tumor implantation. The highest rIL-12 dosages tested (200-500 ng) resulted in a median survival of 38 and 34 days, respectively, and mice treated with 50 ng rIL-12 had a median survival of 27 days. The tumor regression in the surviving animals treated with high doses of 200 ng and 500 ng rIL-12 was confirmed with H&E staining and compared to other rIL-12 treatment dosages and sham controls (FIG. 1D). When comparing our findings to the study conducted by Chiocca et al. 2022, a clinical trial using local expression of IL-12 to treat recurrent GB patients, our 50 ng dose conditions closely resemble their results (Chiocca et al., 2022). In their study, none of the patients survived; however, they observed a discernible benefit from the therapy. Hence, the i.e. administration of 50 ng of rIL-12 was deemed the optimal model for the currently achievable therapeutic effect of IL-12 in GB patients and for conducting subsequent experiments in this study.

### Example 3: IL-12 Perception by the GB Tumor Microenvironment

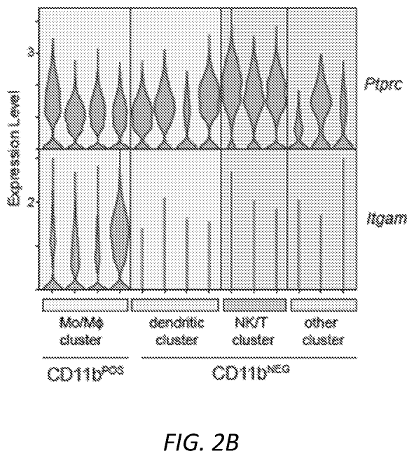

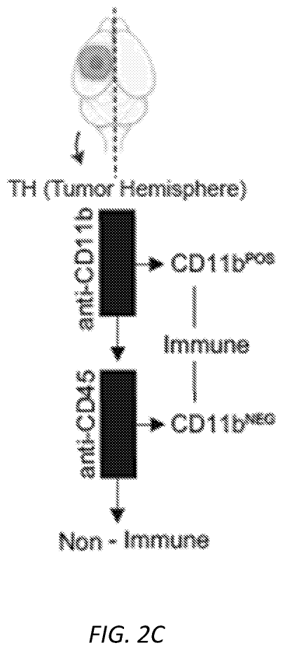

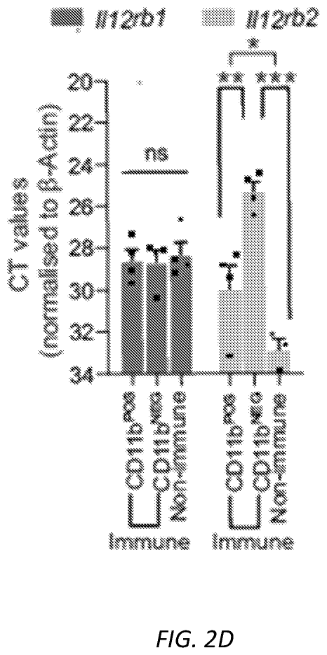

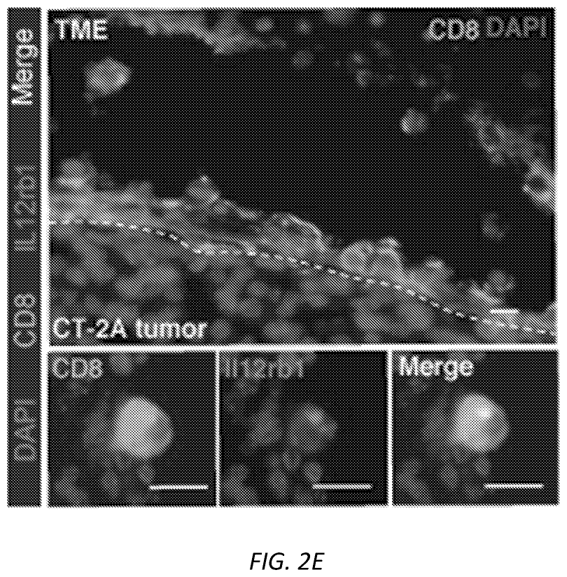

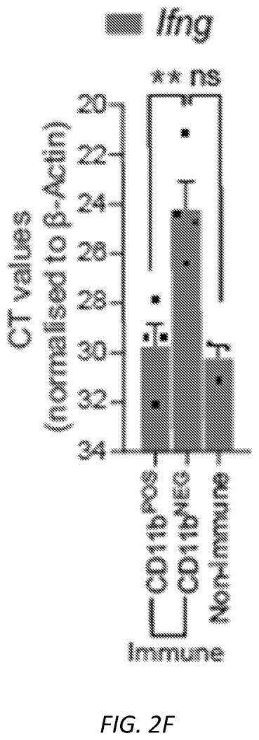

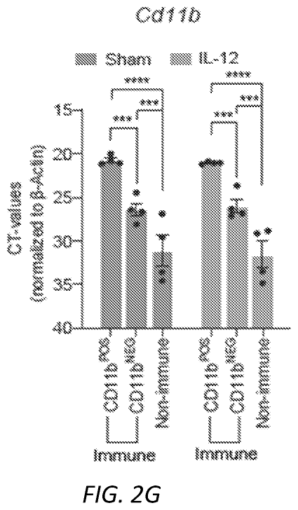

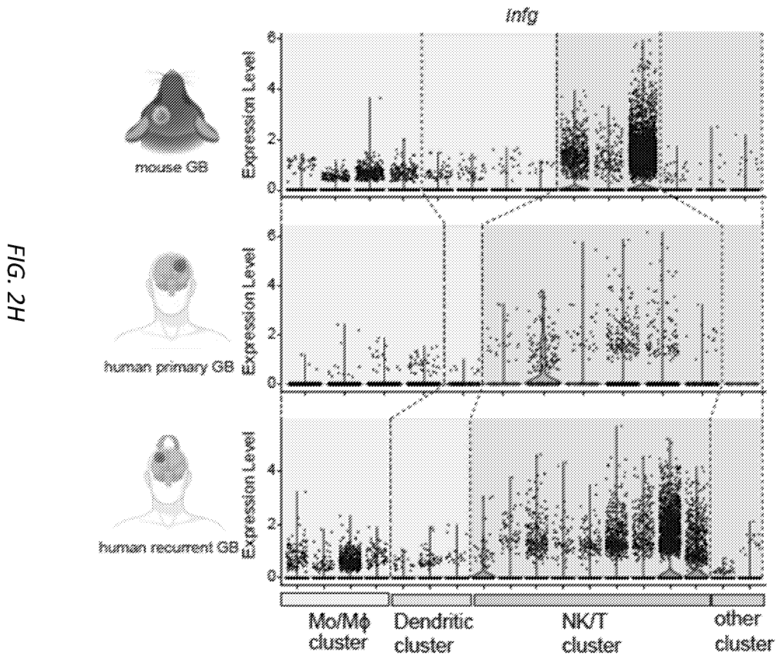

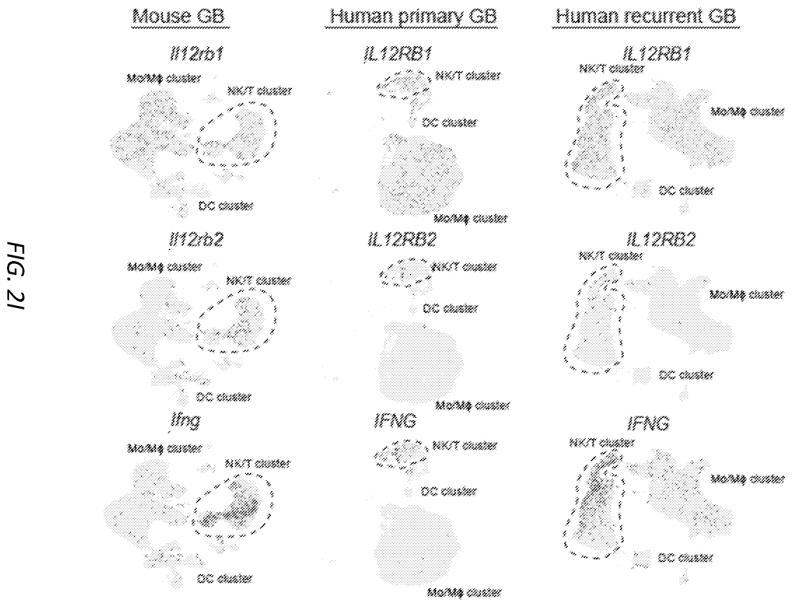

IL-12 triggers pro-inflammatory signaling after binding the dimeric receptor comprised of IL-12 receptor β1 (Il12rb1) and IL-12 receptor β2 (Il12rb2) subunits (Watford et al., 2003). To determine which cells can sense to IL-12 in GB and potentially mediate the IL-12 response in the TME context, we re-analyzed available GB-derived CD45^{POS} single cell RNA sequencing (scRNAseq) datasets (Pombo Antunes et al., 2021) for their expression the Il12rb1 and Il12rb2 genes (FIG. 2A).

To provide a comprehensive overview of Il12rb1/2 transcript expression, we simplified data representation further by reducing the dataset into four classes: monocytes/macrophage (Mo/Mφ) cells, dendritic cells (DCs), natural killer/T cells (NK/T) and other cell clusters, such as B cells and mast cells. Each dot in the graph represents a cell in a certain cell cluster, and the Il12rb1/2 gene expression level per cell is annotated by the y-axis. In mouse GB, transcriptome analysis revealed that the NK/T cluster exhibited the highest expression of Il12rb2 compared to the other clusters (FIG. 2A). Interestingly, when comparing the primary GB patient dataset with the recurrent GB patient dataset, there was an increased presence of IL12RB2-expressing cells observed in the NK/T cell cluster. IL12RB1 was also highly expressed in the NK/T cluster and had a higher number of positive cells compared to the IL12RB2. Notably, the mouse GB dataset exhibited an Il12rb1/2 expression profile that resembled that of recurrent human GBM, particularly when considering the expression levels in the NK/T and DC clusters (FIG. 2A). In each dataset, expression of Il12rb1 was observed in the Mo/Mφ cluster, and to lesser extend in the DC cluster, i.e., both in the mouse model and the human GB samples.

To study IL-12 receptor mediated tumor immunity by the NK/T cluster, we tried to enrich for these IL-12R-rich cells in GB mouse samples (CT-2A model).—Based on scRNAseq analysis, expression of CD45 (Ptprc) was uniform across all immune cell type clusters, whereas CD11b (Itgam) was highly expressed in the Mo/Mφ cluster (FIG. 2B). To validate these in silico findings, we enzymatically dissociated the tumor hemisphere (TH) from CT-2A-Fluc injected mice into single cells and enriched CD11b^{POS} cells with anti-CD11b beads (CD11b^{POS} in FIG. 2C). The resulting CD11b-depleted solution was exposed to anti-CD45 beads-loaded columns to enrich for the remaining immune cells (CD11b^{NEG} in FIG. 2C). We determined the expression of Cd11b in these tumor-brain tissue single cell populations and found that it was significantly higher in CD11b^{POS} populations as compared to CD11b^{NEG} and non-immune cell populations. The expression level of CD11b was consistent in both sham and rIL-12 treatment groups (FIG. 2G). Furthermore, in both CD11b^{POS} and CD11b^{NEG} (CD45^{POS}) enriched GB-brain fractions we also determined Il12rb1/2 expression (FIG. 2D). Il12rb1 transcript levels were expressed at similar levels among these populations. However, Il12rb2 expression was higher in CD11b^{NEG} (CD45^{POS}) immune cells as compared to the CD11b^{POS} cells and the non-immune cells.

We know from the VDX clinical trials, that CD8^{POS} T cells were being activated by IL-12 while CD4^{POS} T cells were not (Chiocca et al., 2019). Therefore, we investigated if CD8 T cells could perceive IL-12 in the GB microenvironment and mediate antitumor immunity. IL-12 receptor expression in tumor-associated immune cells was confirmed with immunohistochemistry which demonstrated that CD8^{POS} T cells express Il12rb1 protein (FIG. 2E). Furthermore, interferon-γ (IFN-γ), a signature for cytotoxic activity in the NK/T cell cluster (FIG. 2H), was verified in each of the isolated CD11b^{POS}, CD11b^{NEG} (CD45^{POS}), and CD45^{NEG} fractions of the tumor (ipsilateral) hemisphere. The levels of Ifng had a comparable profile as Il12rb1/2 and were highest in CD11b^{NEG} immune cells as compared to CD11b^{POS} immune cells and non-immune cells (FIG. 2F).

Taken together, our scRNAseq and RT-qPCR analysis provides evidence that IFN-γ and Il12rb1/2 expression are both elevated in the NK/T cluster, while our immunohistochemistry indicates the potential for CD8^{POS} T cells within that NK/T cluster to have an active role in rIL-12-mediated antitumor immunity (FIG. 2I). Of note, the levels of IFN-γ in the NK/T cluster in a mouse GB tumor were more comparable to recurrent human GB tumor, rather than primary human GB tumor (FIG. 2J).

### Example 4: Intratumoral rIL-12 Supports CD8^{POS} T-Lymphocyte Recruitment and Differentiation Towards an Effector-Like Phenotype

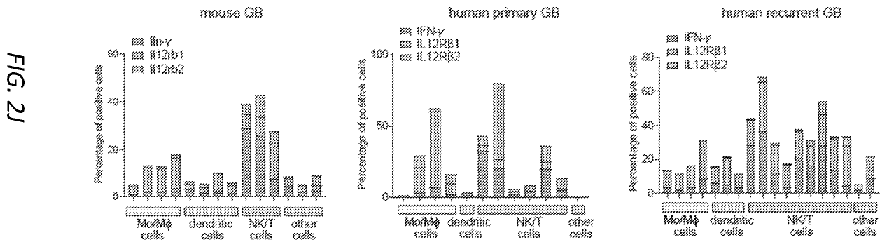

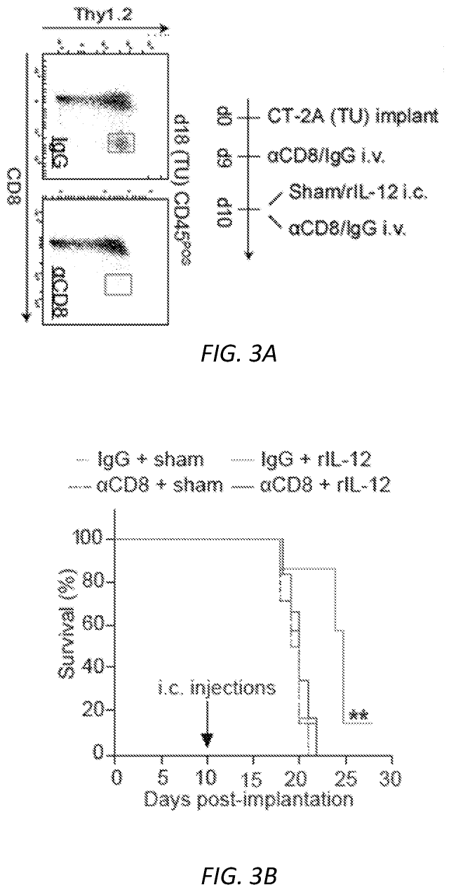

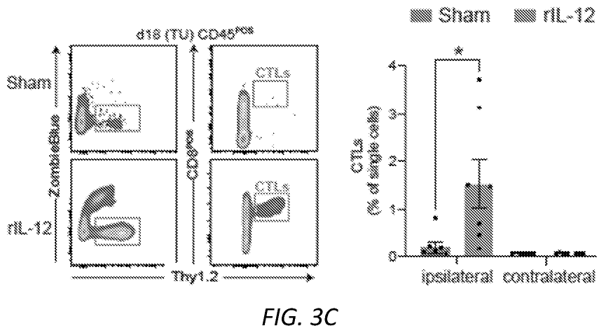

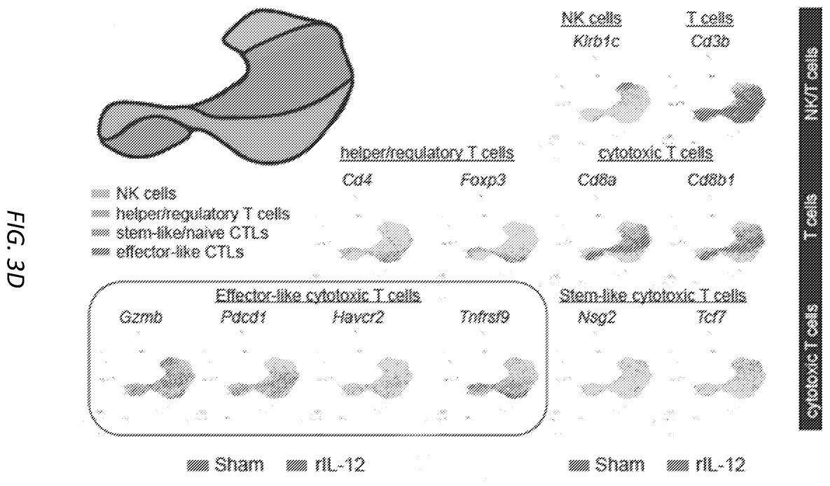

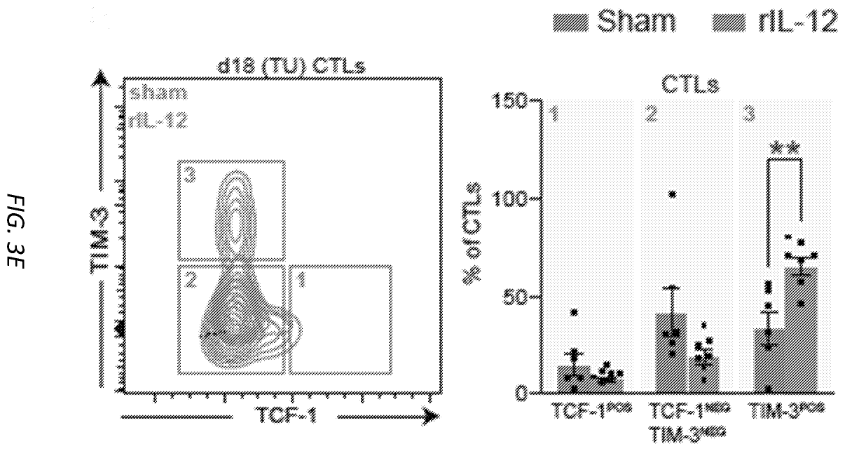

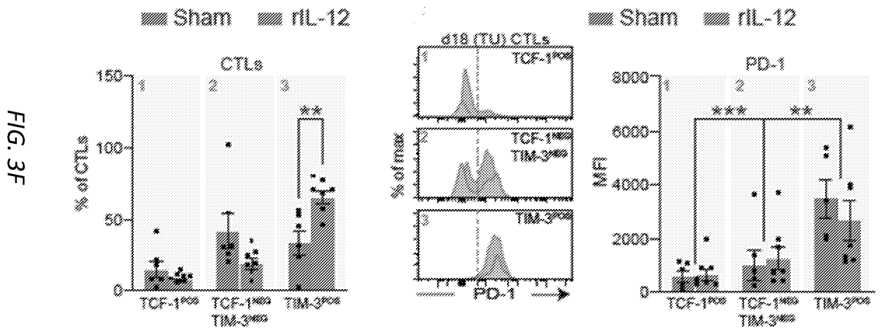

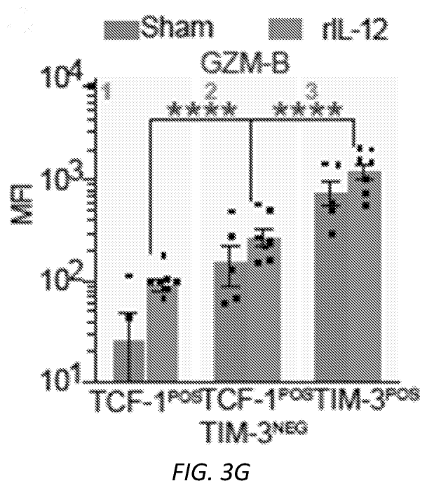

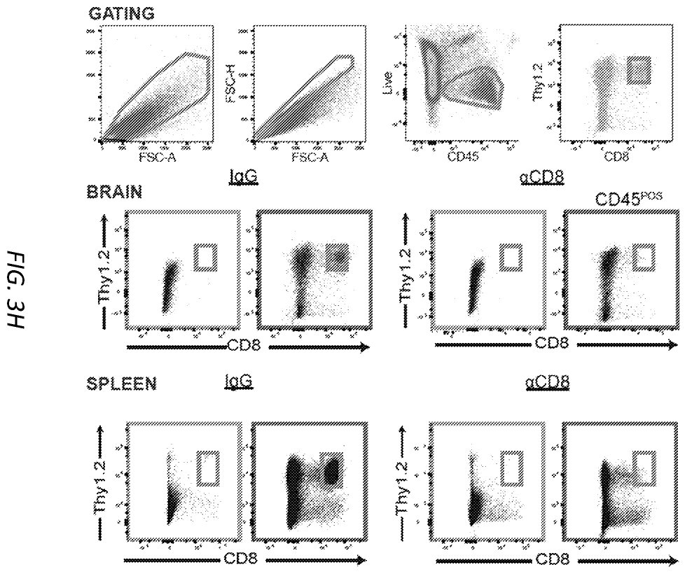

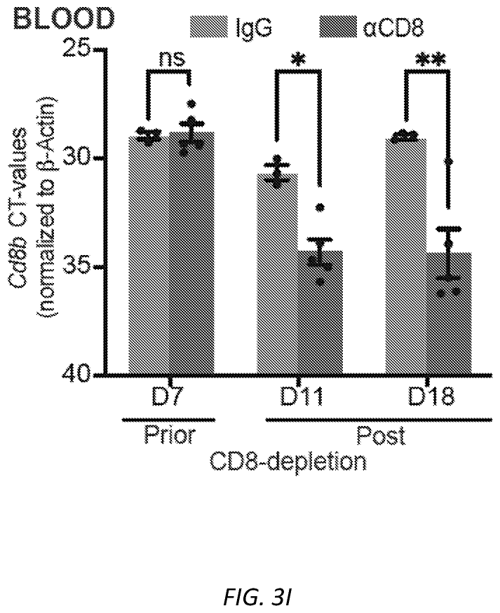

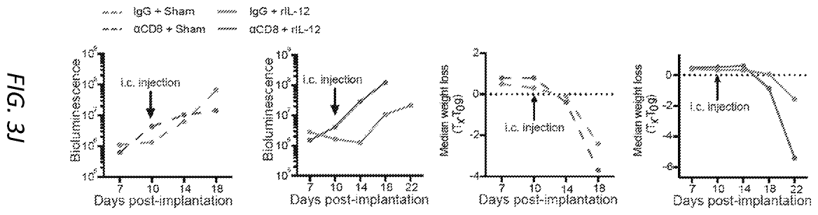

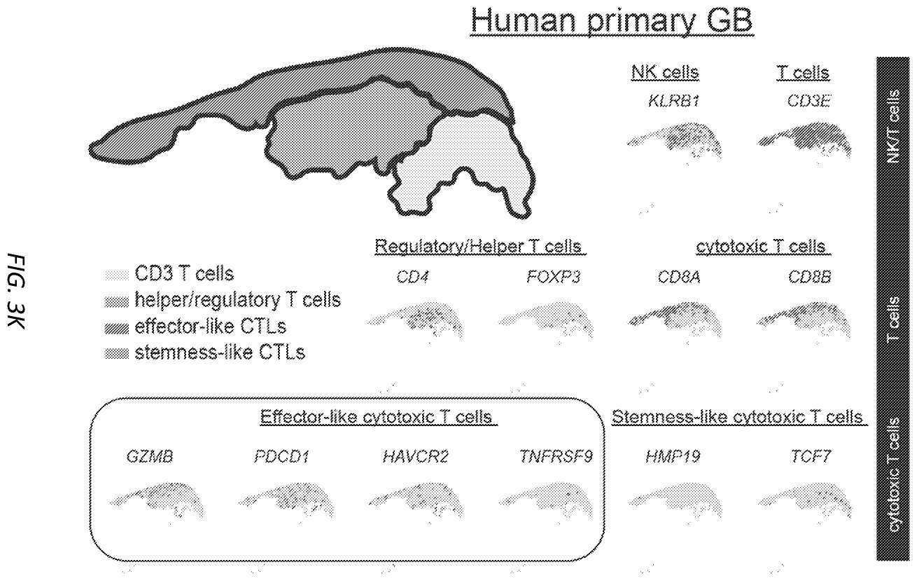

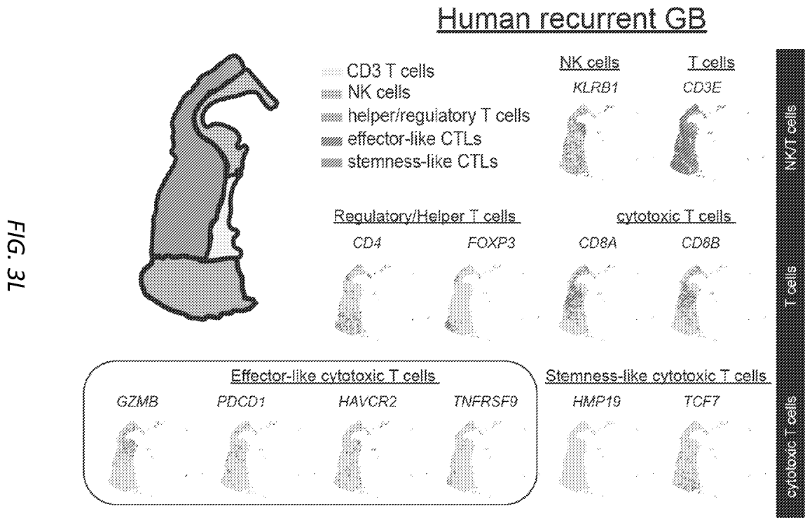

Failure of immunotherapy in GB is in part due to immune suppressive TME-driven T cell dysfunction defined by reduced proliferation capacity, lower production of effector cytokines, and sustained expression of inhibitory receptors (Watowich et al., 2023). Reactivating hypo/dysfunctional T cells has been a major interest in increasing antitumor immunity. Recent studies revealed hypofunctional cytotoxic T lymphocyte (CTL) populations with tumor antigen recognition potential in immunological effector sites, including tumors (Siddiqui et al., 2019). To address whether tumor-reactive CD8^{POS} CTLs in the TME are recruited from the blood facilitating the rIL-12-mediated increase of survival, we depleted CTLs systemically prior to rIL-12 treatment. To that end, we treated our syngeneic GB model with intravenous (i.v.) injections of anti-CD8 antibodies and i.e. injections of rIL-12 on day 10 (FIG. 3A). Compared to control IgG, anti-CD8 injections were able to deplete CTLs in the CD11b^{NEG} (CD45^{POS}) cell fractions isolated from whole brain using anti-CD11b and anti-CD45 beads of an rIL-12 treated CT-2A-Fluc tumor (FIGS. 3A and 3H). As expected, the CD8 T cells were not detected in the CD11b^{POS} cell fractions of the same brains (FIG. 3H). We also demonstrated that CD8 T cell depletion was not merely restricted to the brain but could also be observed in the spleen and blood (FIGS. 3H and 3I). In the latter, we found sustained systemic depletion, as Cd8b transcript levels significantly decrease in retro-orbital blood samples at day 11 and 18 post-tumor implantation after the last i.v. injection with anti-CD8, while this was not the case at day 7 two days prior to the first injection with anti-CD8. To test whether CTL recruitment affects IL-12-induced anti-tumor activity, we measured survival of mice treated i.e. with sham or rIL-12 post-tumor implantation and anti-CD8 administration (FIG. 3B). Remarkably, the CTL depletion nullified the rIL-12-induced increase in GB mice survival to similar levels as sham treated GB mice. This was confirmed by increased tumor growth and a reduction in weight with anti-CD8 as compared to IgG control in rIL-12 treated GB mice (FIG. 3J). No differences were seen based on survival or tumor growth between IgG and anti-CD8 treatments in mice treated with sham control, indicating that a small number of CTLs are recruited into the CT-2A tumor brain without an IL-12 stimulus (FIG. 3J). We further analyzed the CD11b^{NEG} (CD45^{POS}) cell fraction of an rIL-12 stimulated CT-2A tumor by using flow cytometry (FIG. 3C). In sham treated GB mice, a lower number of CTLs were observed in the brain, while a higher number of CTLs could be seen with rIL-12 treatment. The higher number of CTLs with rIL-12 treatment, potentially resulting from increased CTL recruitment or proliferation, was notably restricted to the tumor site, and was not detected in the contralateral tumor hemisphere (FIG. 3C).

Although CTL numbers increased at the GB site, that does not guarantee their anti-tumor activity. CTL responses against cancer are maintained by stem-like memory cells that self-renew and give rise to effector-like cells that harbor cytotoxic functions against tumor cells (Di Pilato et al., 2021). We reassessed the NK/T cluster from our scRNAseq datasets, where we previously identified Il12rb1/2 and Ifnγ expression, to verify whether findings made in other types of tumors also apply to GB (FIGS. 3D and 3K and 3L). First, we distinguished NK cells from T cells based on Klrb1c and Cd3b expression, respectively. Then, helper and regulatory T cells, expressing Cd4 and Foxp3 genes, were discriminated from the CTLs marked by Cd8a b expression. In the CTL cluster, we observed naïve and stem-like CTLs, expressing Tcf7 (encoding for TCF-1) and Nsg2 genes, that were different from the CTLs with an effector-like phenotype expressing Havcr2 (encoding for TIM-3), Pdcd1 (encoding for PD-1), Gzmb (encoding for granzyme-B) and Tnfrsf9 (encoding for 4-1BB) (Hudson et al., 2019; Utzschneider et al., 2016) (FIGS. 3D and 3K and 3L). We used this information to study CTL maturation in GB in response to rIL-12. Eighteen days post-tumor implantation, the CD11b^{NEG} (CD45^{POS}) cell fraction of an rIL-12 or sham treated CT-2A tumor was analyzed with flow cytometry (FIGS. 3E and 3F). We observed eight days post-rIL-12 treatment, a shift from TCF-1^{NEG}TIM-3^{NEG} to TCF-1^{NEG}TIM-3^{POS} CTLs as compared to sham indicating a transition from a stem-like to an effector-like state. We documented this finding by examining the additional activation marker PD-1 on CTLs (Honda et al., 2014). We observed that TIM-3 expression correlated with a marked increase in PD-1 levels and number of PD-1^{POS} cells in the TME (FIGS. 3F and 3O). PD-1 levels were highest in TIM-3^{POS} effector-like CTLs, compared to TIM-3^{NEG}TCF-1^{POS} and TIM-3^{NEG}TCF-1^{NEG} cells. Similar to PD-1, GZM-B gradually increased along the TCF-1-TIM-3 differentiation axis. We observed that TCF-1^{NEG}TIM-3^{POS} CTLs with high levels of PD-1 also express high levels of cytotoxic GZM-B, accentuating cytotoxic activity in differentiated CTLs (FIG. 3G). Overall, rIL-12 treatment mainly influenced activation and late differentiation markers such as Pdcd1, Gzmb, and Cd101, while this was less prominent for Havcr2 and Tcf-7 expression in the CD11b^{NEG} (CD45^{POS}) cell fraction of an rIL-12 or sham treated CT-2A tumor hemisphere (FIG. 3N). Of note, CD-101 is a marker often associated with hypofunctional T cells, indicating CTLs that underwent progressive transition from a stem-like to effector-like status (FIGS. 3L and 3N).

In summary, the above data shows that the rIL-12 antitumoral effect against GB is due to a local accumulation of CTLs. In parallel, CTLs at the tumor site injected with rIL-12 are enriched for more activated, TIM-3^{POS} cells, suggesting enhanced progression of stem-like toward cytotoxic effector-like and eventually hypofunctional cells.

### Example 5: Evaluating IL-12b-Expressing Dendritic Cells in TME for Co-Stimulatory CTL Factors

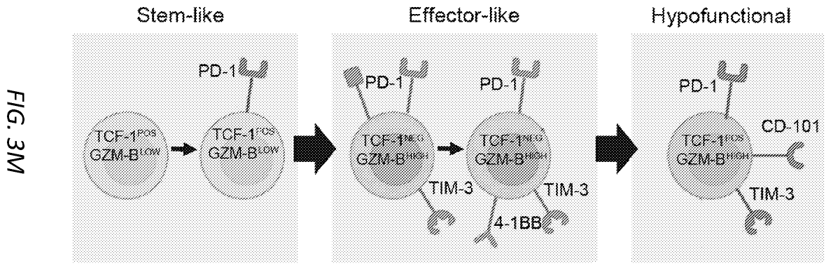

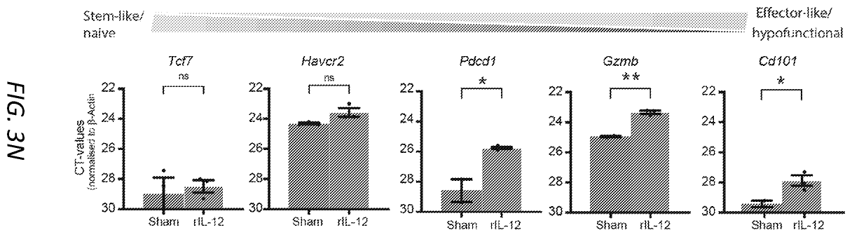

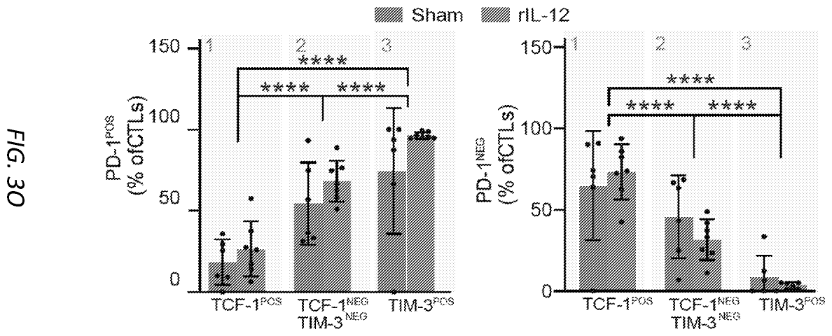

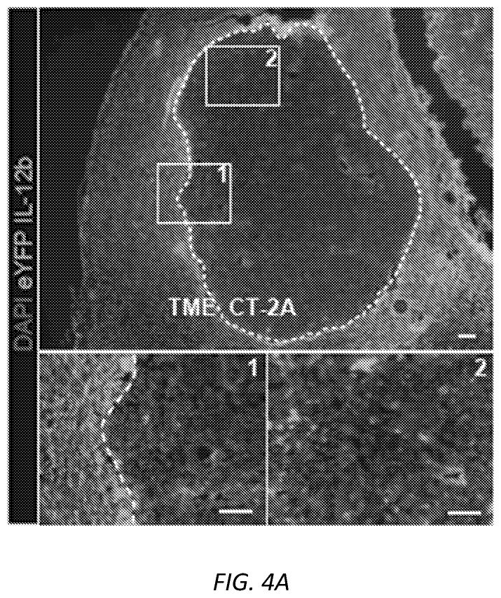

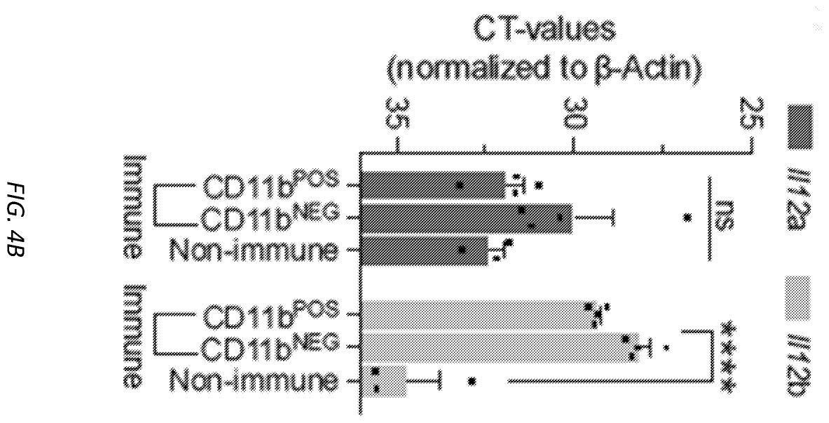

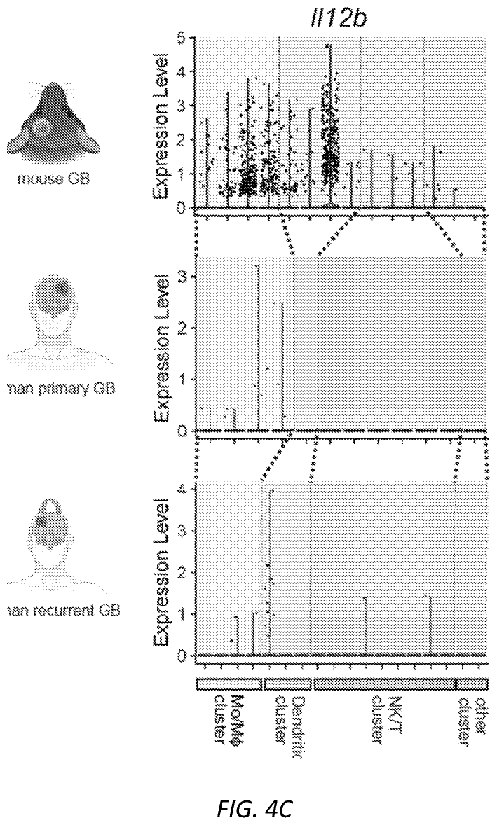

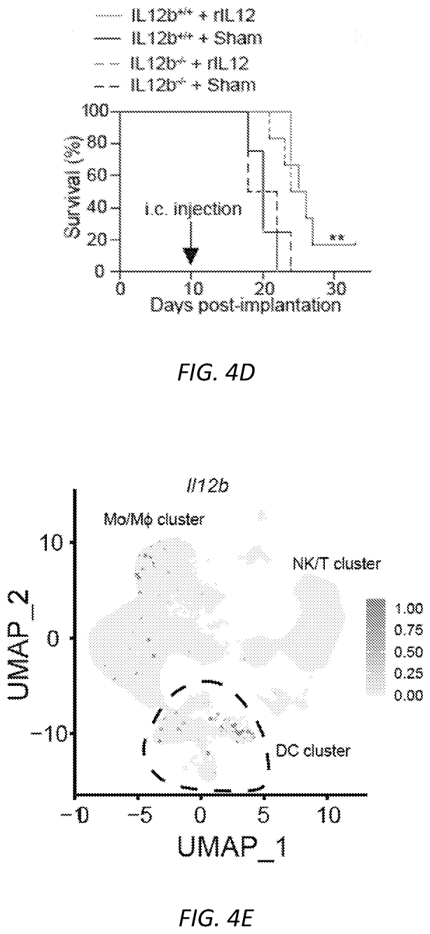

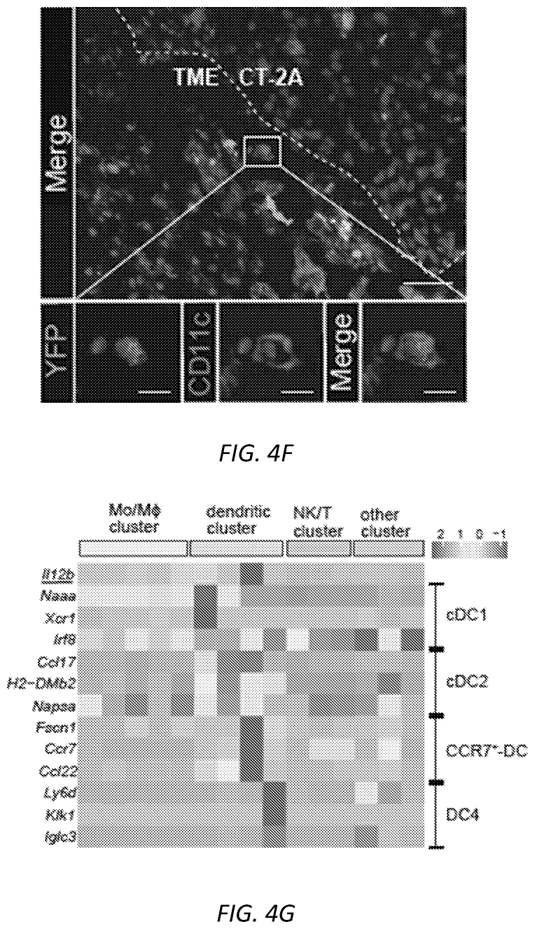

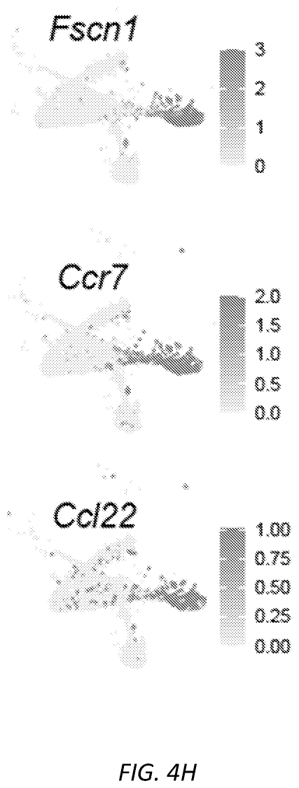

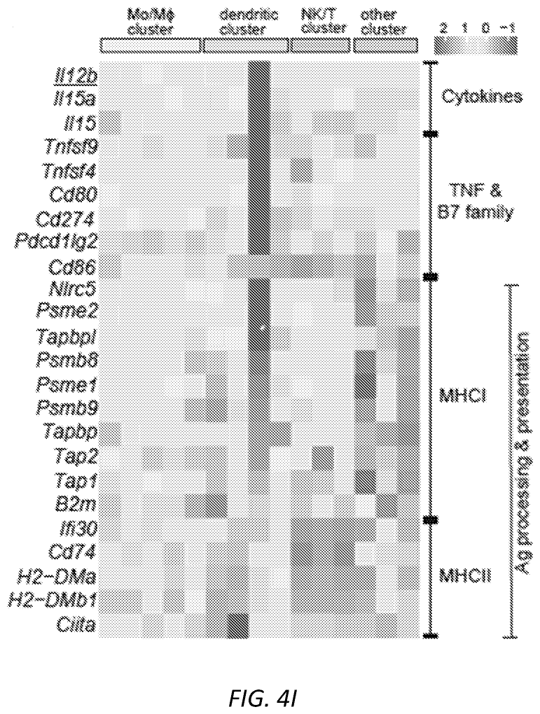

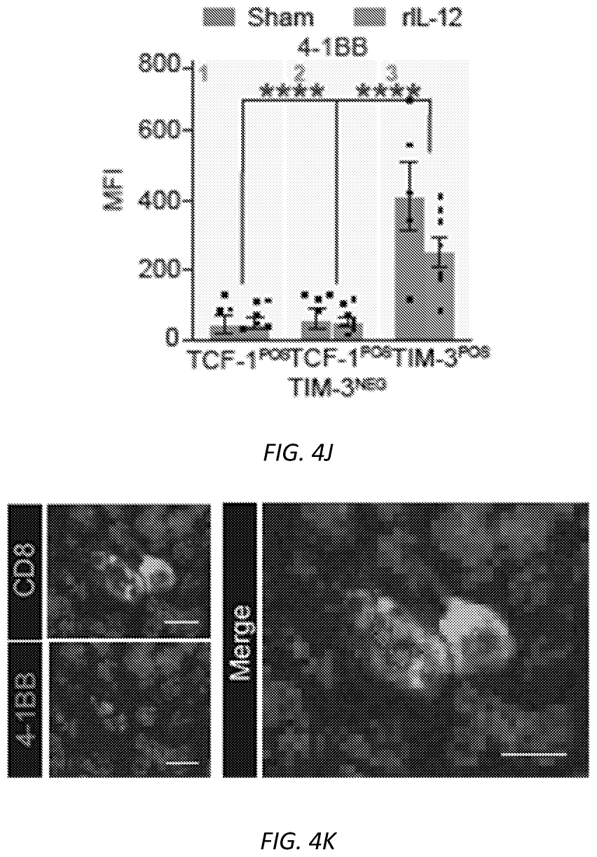

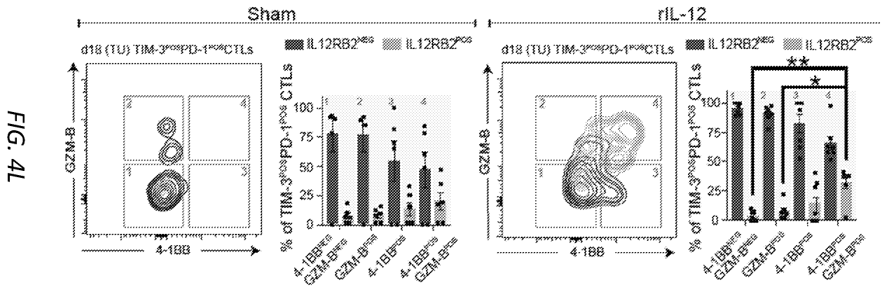

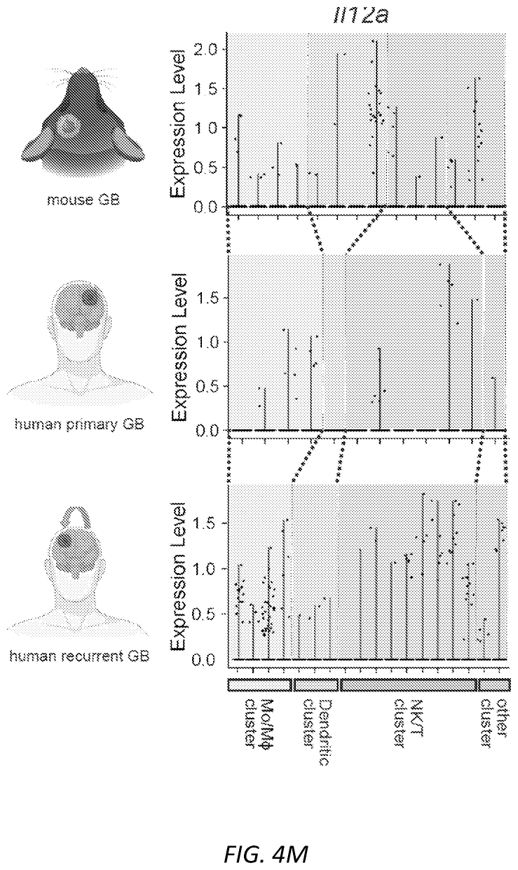

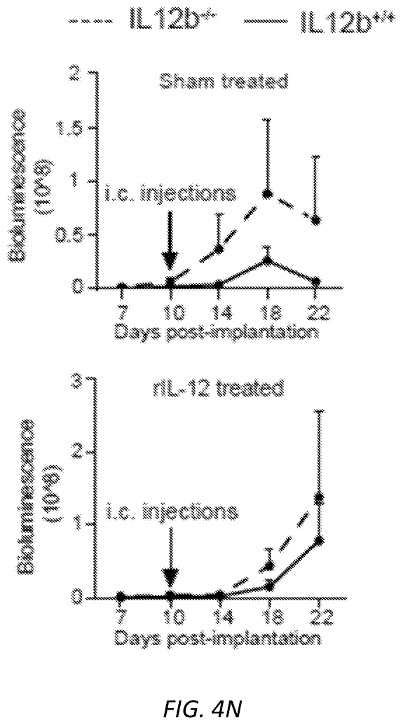

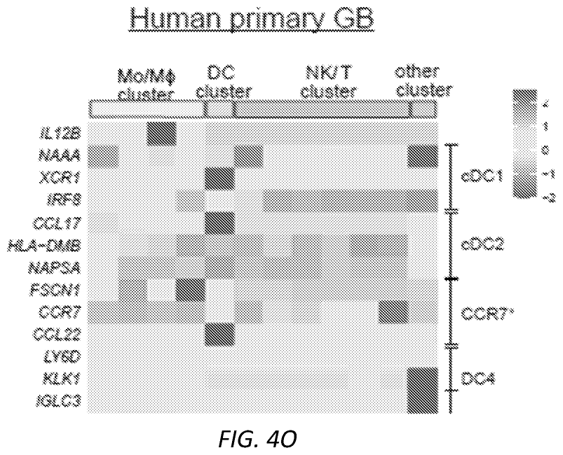

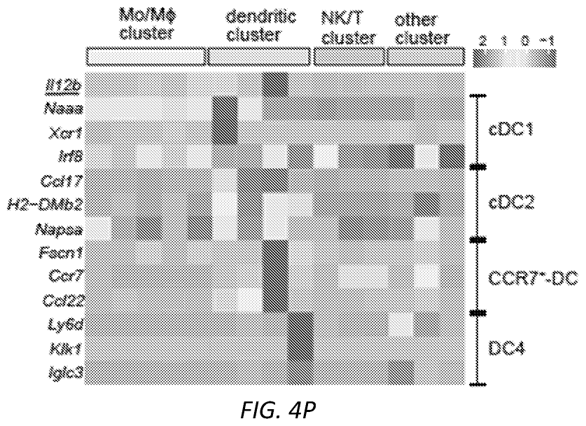

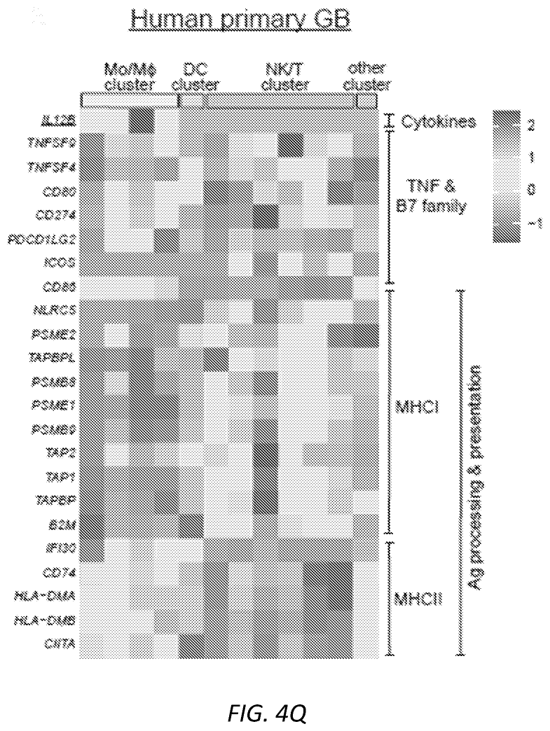

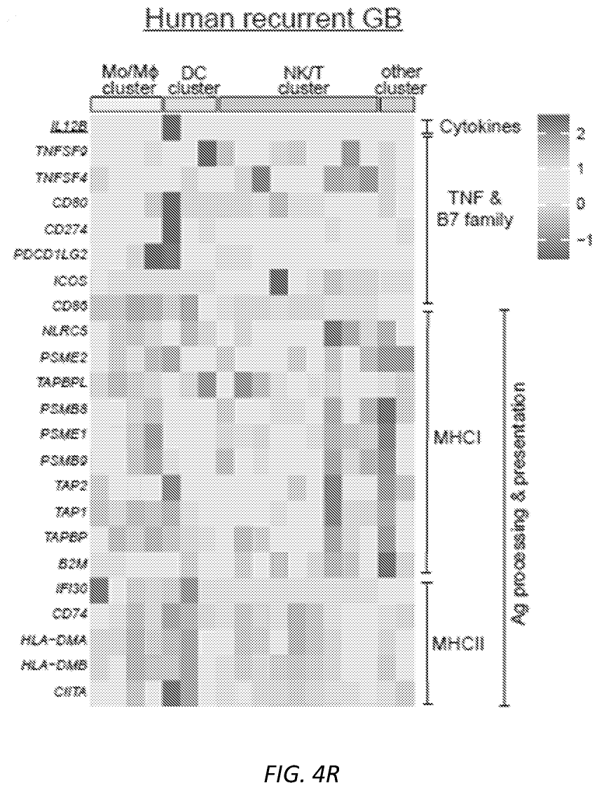

To test whether rIL-12 is actively engaged in immunomodulation at the TME, CT-2A cells were implanted in IL-12b-YFP reporter mice (Reinhardt et al., 2006) (FIG. 4A). YFP^{POS} cells were distributed in the peritumoral area and exhibited a relatively limited infiltration of the tumor parenchyma (white dotted line). The CT-2A tumor was fractionated into CD11b^{POS/NEG} (CD45^{POS}) cells and demonstrating that Il12b transcripts were predominantly expressed in the immune compartment and not in the non-immune cells that comprise tumor cells or host-derived CD45^{NEG} cells (FIG. 4B). Validation of our findings was supported by scRNAseq analysis of mouse GB using the GL261 model, which showed the expression Il12b in Mo/Mφ cluster and the DC cluster (FIG. 4C). The transcription of Il12a did not exhibit significant differences among cell types (FIGS. 4B and 4L).

When comparing the expression levels of Il12b in the scRNAseq dataset obtained from the GB mouse model with datasets from GB patients, significant disparities were evident, particularly with a considerably limited expression observed in patients (FIG. 4C). To assess if our observations are translatable to lower endogenous IL-12 induction observed in GB patients, we treated Il12b-mice (Magram et al., 1996) with rIL-12 (FIGS. 4D and 4M). rIL-12 treatment still significantly improved median overall survival, independent of endogenous IL-12 expression (FIGS. 4D and 4M).

We demonstrated that ectopic IL-12 is aiding survival and potentially overcoming the need for endogenous IL-12b producer cells to induce anti-tumor immunity properly. However, the latter cell type would still be able to aid in retrieving markers that could stimulate IL-12 immunity. In our UMAP projection of mouse GB, we detected Il12b expressing cells in a dense cluster within a DC subcluster (FIG. 4E). DCs play a crucial role in eliciting differentiation and activation of tumor-reactive CTLs (Fu and Jiang, 2018), however in a “cold” tumor, regulatory DCs fail to support T cell activation due to the high expression of inhibitory factors overruling their co-stimulatory factors or due to the small number of DCs or T cells at the GB site (Maier et al., 2020). We identified DCs at the GB TME with the CD11c marker and demonstrated that they co-expressed IL-12, shown by YFP in IL-12b-YFP reporter mice (FIG. 4F). However, the number of CD11c^{POS} IL-12b POS at the GB site was low. Considering this, we asked if IL-12 expressing DCs harbor stimulatory factors that navigate CTLs activation at the GB site. We mapped the top highly expressed genes of each DC subcluster and compared it to the Il12b expression subcluster (FIG. 4G). The profiles of Fscn1, Ccr7, and Ccl22 transcripts defining the CCR7^{POS}-DC subcluster corresponded to highest Il12b expression (FIG. 4H). Similar conclusions were derived from the human recurrent GB dataset, while this was not the case for human primary GB that contained very small numbers of DCs (FIGS. 4N and 4O). We cross-referenced factors produced by CCR7^{POS}-DCs that support CTL proliferation and survival in melanoma models with our GB model (Di Pilato et al., 2021) (FIG. 4I). In our analysis, high levels of the TNF superfamily genes including co-stimulatory CTL factor Tnfsf9 (encoding for 4-1BBL), and genes important for MHCI/II antigen processing and presentation to CTLs were expressed in CCR7^{POS}-DCs in mouse GB. We confirmed that Tnfsf9 is also expressed by DCs in recurrent human tumors, but not in primary GB (FIGS. 4P and 4Q). Of note, high levels of Cd274 (encoding for PD-L1) in mouse and human GB were also observed, indicating that next to co-stimulatory factors, inhibitory factors have the potential to interact with CTLs when forming an immune synapse with CCR7^{POS}-DCs.

Given the limited availability of CCR7^{POS} DCs, leading to minimal IL-12 and 4-1BBL levels at the GB site, and considering the inhibitory influence of factors like PD-L1 on tumor-reactive CTL, our investigation extended to exploring the potential substitution of not only IL-12 but also 4-1BBL. We tested whether a subset of tumor-reactive CTLs in the TME of mouse GB can perceive the co-stimulatory CTL factor 4-1BBL during rIL-12-stimulation. As noted above, treatment with rIL-12 results in an increased number of effector-like CTLs within the TME but also changes the activity of CTLs. We were wondering whether rIL-12 therapy would still retain 4-1BB receptor-expressing effector-like CTLs within a GB tumor (FIG. 3D). We confirmed by flow cytometry that 4-1BB CTLs were only detected in TCF-1^{NEG}TIM-3^{POS} CTLs (FIG. 4J). Immunohistochemistry showed 4-1BB co-localized with CD8^{POS} CTLs at the tumor border upon rIL-12 treatment (FIG. 4K). Tumor site specific expression was confirmed with flow cytometry in the tumor hemisphere, contralateral hemisphere, and spleen (FIG. 4R). Lastly, we examined if rIL-12 and 4-1BBL could act on the same subset of CTLs (FIG. 4L). In sham treated CT-2A tumors, small numbers of PD-1^{POS}TIM-3^{POS} CTLs were found expressing low levels of either IL-12Rb2 and/or 4-1BB. However, upon rIL-12 treatment, we observed a shift in 4-1BB positivity in the TME. This shift was induced by an increase in IL12rb2^{POS} CTLs which were predominantly in 4-1BB^{POS}GZM-B^{NEG} and 4-1BB^{POS}GZM-B^{POS} CTLs and not 4-1BB^{NEG}GZM-B^{POS} CTLs or 4-1BB^{NEG}GZM-B^{NEG} CTLs. IL12rb2^{NEG} CTLs (black) in rIL-12 treated brains were mainly in GZM-B^{POS} alone CTLs or 4-1BB^{NEG}GZM-B^{NEG} CTLs. The levels of IL12rb2 in the different populations corroborated this data (FIG. 4S).

Taken together, our findings propose that CCR7^{POS} DCs could potentially function as a reservoir of endogenous IL-12b and 4-1BBL expression. Augmenting the TME with both immune stimulants could potentially substitute the CCR7^{POS} DC function and enhance the activity or expansion of a CTL subset expressing both IL-12R and 4-1BB. This approach may thereby delay the terminal differentiation and decline of cytotoxic function of CTLs observed with sole rIL-12 treatment.

### Example 6: Anti-Glioma Immunity Activated by Combined 4-1BBL and rIL-12 Immune Stimuli Increased Survival of Tumor-Bearing Mice

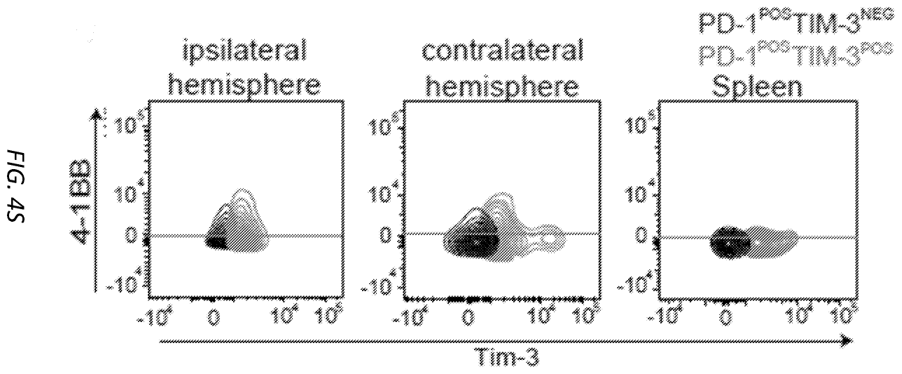

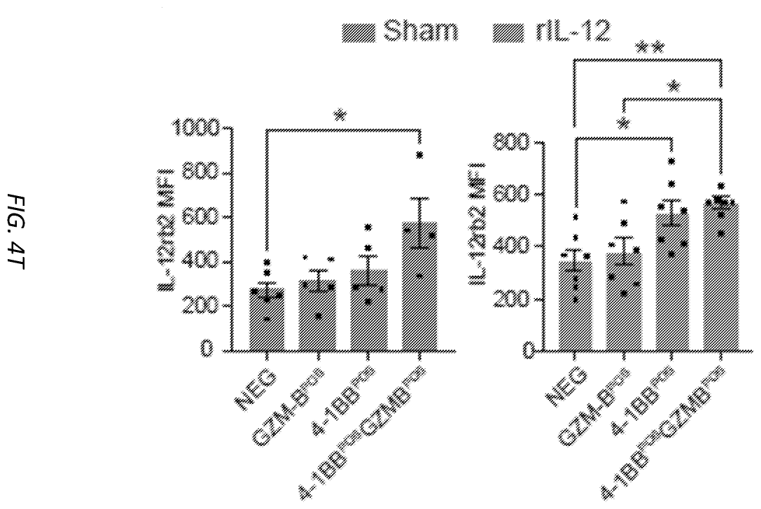

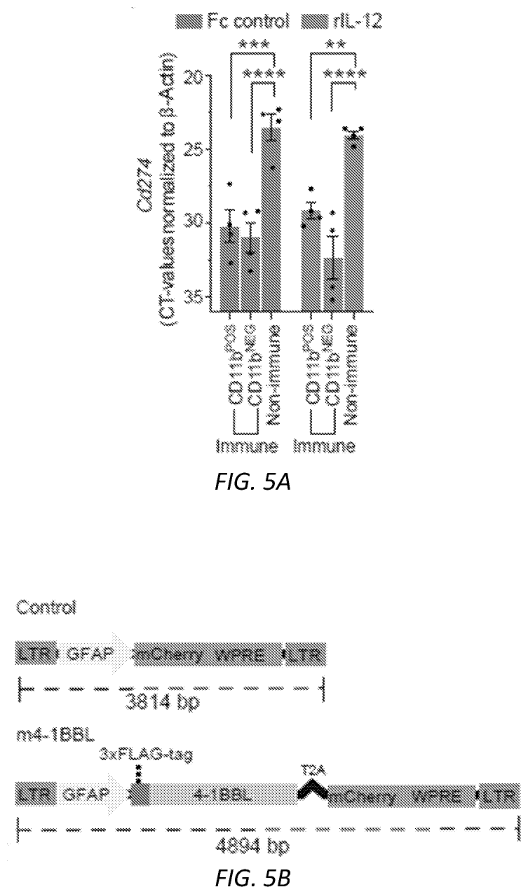

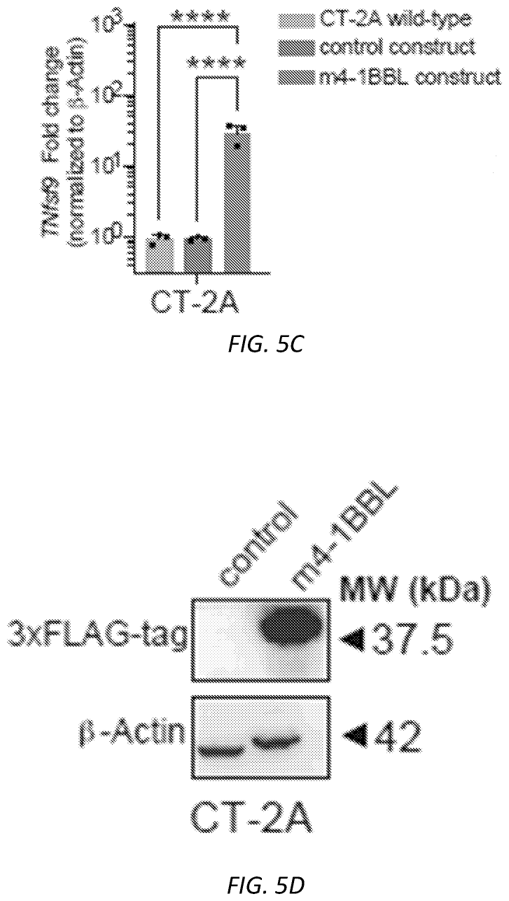

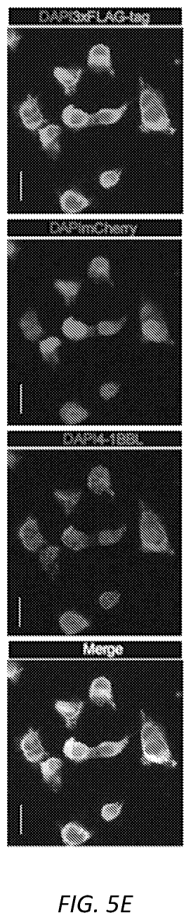

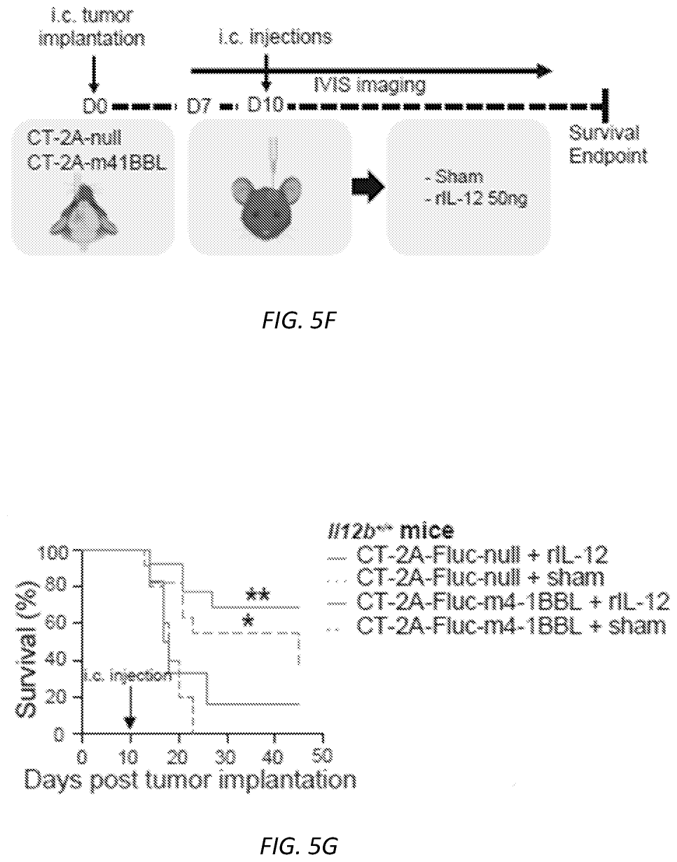

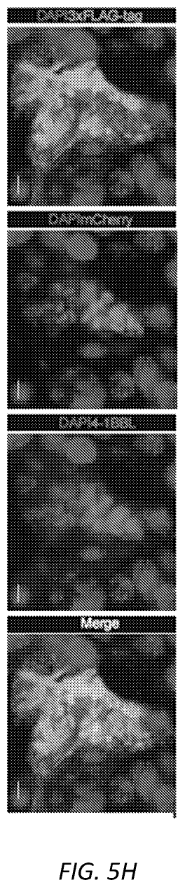

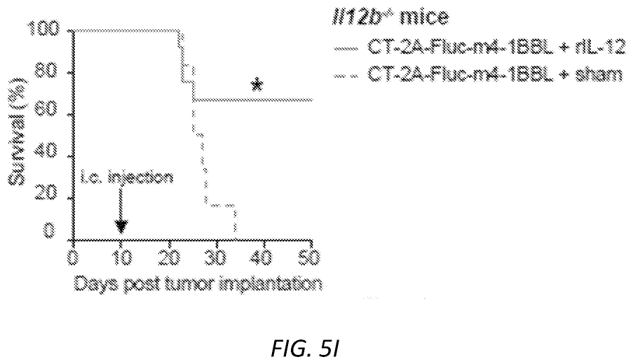

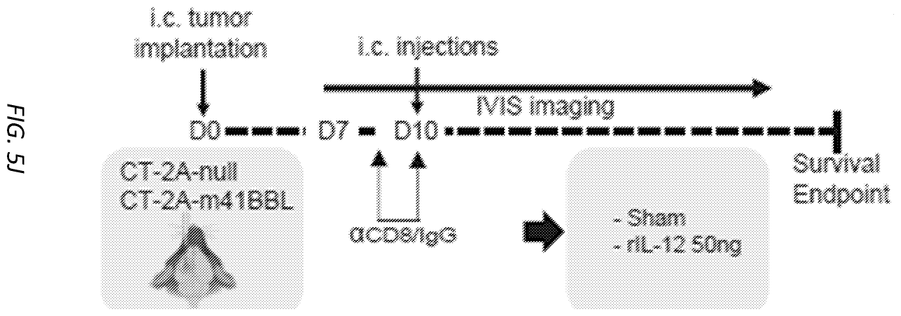

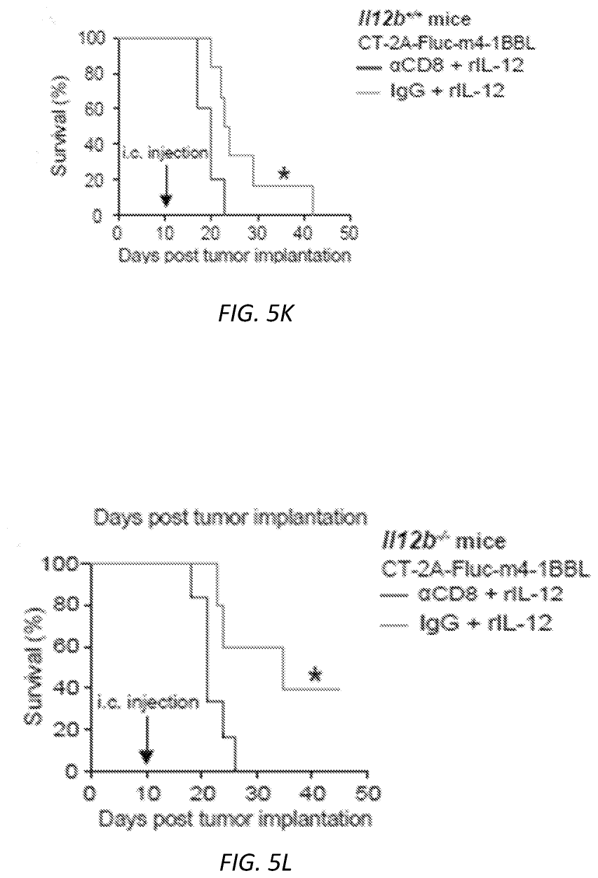

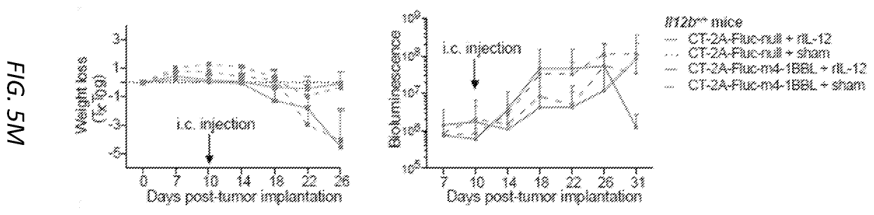

Since rIL-12 treated tumor are associated with tumor-reactive 4-1BB^{POS}CTLs, we tested whether 4-1BBL, the ligand of 4-1BB (Goodwin et al., 1993), could extend CTL functionality at the tumor site. Based on the VDX clinical trials we know that CTL stimulation must overcome the PD-L1-rich GB environment (Chiocca et al., 2019). We screened our rIL-12-stimulated GB mouse model for PD-L1 and confirmed high expression of Cd274 (the PD-L1 gene) in the non-immune cell fraction containing CT-2A tumor cells (FIG. 5A). To express 4-1BBL at the tumor site, we designed a lentiviral vector (LVV) construct that encoded murine 4-1BBL (m4-1BBL) (FIG. 5B). To differentiate between endogenous and recombinant 4-1BBL in our mouse model, we fused m4-1BBL to a 3×FLAG-tag on the intracellular facing side of this single-pass transmembrane protein. A T2A protease cleavage site separating mCherry fluorescent reporter transgene was included to visualize LVV transduction of cells without anti-3×FLAG-tag staining. An inactive mimic LVV encoding mCherry lacking the 3×FLAG-tag and 4-1BBL was generated as control. Post-LVV transduction, a stable CT-2A-Fluc-m4-1BBL cell line was generated, and RT-qPCR analysis confirmed high TNfsf9 (encoding for 4-1BBL) expression compared to wild-type (WT) CT-2A glioma cells and those transduced with the control vector (CT-2A-Fluc-null) (FIG. 5C). 3×FLAG-tag was detected at the protein level in CT-2A-Fluc-m4-1BBL cells by western blot (FIG. 5D). Transduced cells were sorted for mCherry positive cells prior to implantation in mice. We verified homogenous expression CT-2A-Fluc-m4-1BBL transduced cells by immunocytochemistry and fluorescent microscopy (FIG. 5E). mCherry fluorescence, a marker of our LVV-expression, was seen in every cell and colocalized with anti-3×FLAG-tag and anti-m4-1BBL staining (FIG. 5E).

CT-2A-Fluc-null or CT-2A-Fluc-m4-1BBL cells were implanted i.e. in mice and treated with rIL-12 or sham on day 10 post-implantation (FIG. 5F). Compared to CT-2A-Fluc-null engrafted mice, mice implanted with CT-2A-Fluc-m4-1BBL cells had significantly increased survival, indicating that 4-1BBL at the tumor site extended the lifespan. Remarkably, 70% of rIL-12-treated mice with a CT-2A-Fluc-m4-1BBL tumor survived, suggesting that the co-stimulatory factor 4-1BBL is sufficient to overrule or delay immune suppression at the tumor site (FIG. 5G). We also monitored other parameters such as weight and tumor growth but did not observe significant differences within the timeframe that mice of all groups were alive (FIG. 5M). Intratumoral m4-1BBL expression was confirmed on day 18 post-tumor implantation through immunohistochemistry illustrating co-localization of m4-1BBL and 3×FLAG-tag in CT-2A-Fluc-m4-1BBL tumor-bearing mouse brains (FIG. 5H). Of note, although cells were sorted prior implantation, our m4-1BBL signal was not observed in the whole tumor. We presume this is due to transgene instability in tumor cells or overgrowth by small numbers of non-expressing 4-1BBL contaminant GB cells.

Anti-glioma induced immunity due to m4-1BBL/rIL-12 combination treatment was evaluated in a more GB-patient relevant model without endogenous IL-12. CT-2A-Fluc-m4-1BBL cells were implanted in Il12b^{-/-} mice and treated with either rIL-12 or sham. rIL-12 treated Il12b^{-/-} mice showed a significantly improved median survival of 78 days compared to 26 days for the sham control (FIG. 5I). In addition, significant changes in body weight or tumor growth were observed within the lifespan of the rIL-12 treated mice (FIG. 5N).

We next investigated whether recruitment of CD8^{POS} CTLs was needed for the rIL-12 and 4-1BBL combination therapy. Mice were systemically depleted of CD8^{POS} T cells through i.v. injection of anti-CD8 post-tumor implantation (FIG. 5J). An IgG control was injected according to the anti-CD8 regimen. The effect of CD8 depletion was tested in both Il12b^{+/+} (FIG. 5K) and Il12b^{-/-} (FIG. 5L) mice that were implanted i.e. with CT-2A-Fluc-m4-1BBL and treated with rIL-12. Improved survival of 30 and 35 days was observed for non-depleted (IgG control) compared to the T cell-depleted (anti-CD8) in the Il12b^{+/+} mice and in the Il12b^{-/-} mice, respectively. In line with the survival data, a downward trend was visible based on weight loss and increased tumor growth for CD8 T cell-depleted mice versus IgG control mice (FIGS. 5O and 5P).

In an immune-suppressive environment, our data demonstrated an enhanced effect when combining m4-1BBL with rIL-12, compared to using rIL-12 treatment alone. This combination led to improved recruitment and/or enhanced functionality of tumor reactive CTLs against glioma.

### Example 7: Immuno-Gene Therapy Targeting the GB Environment as an Avenue to Combine 4-1BBL and rIL-12 Interventions

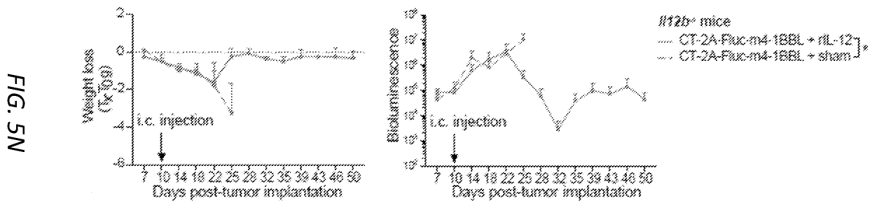

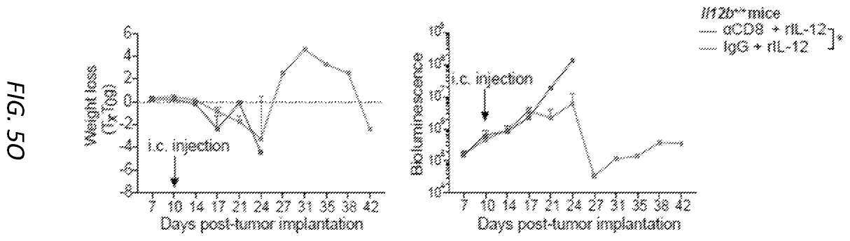

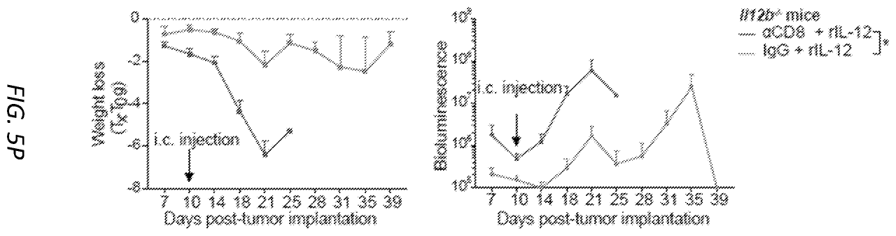

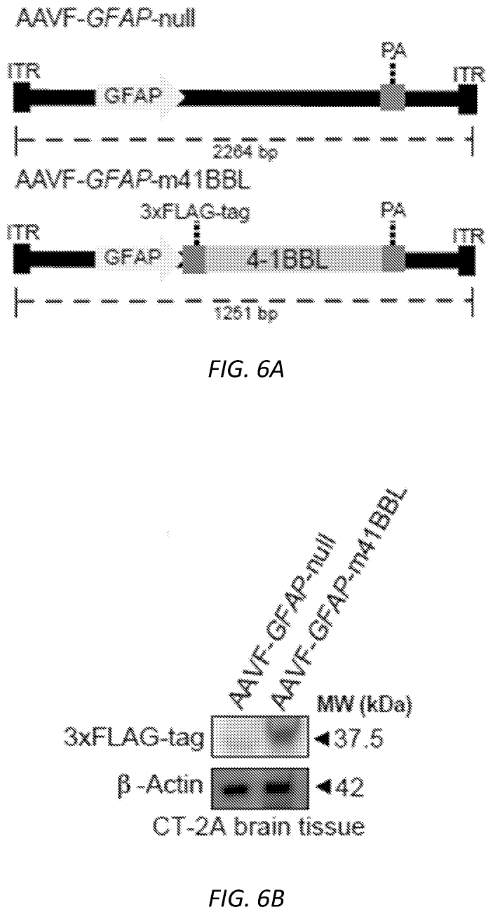

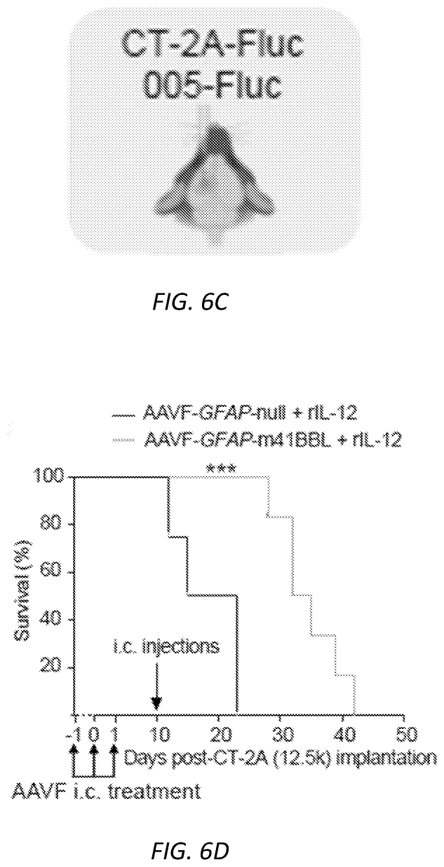

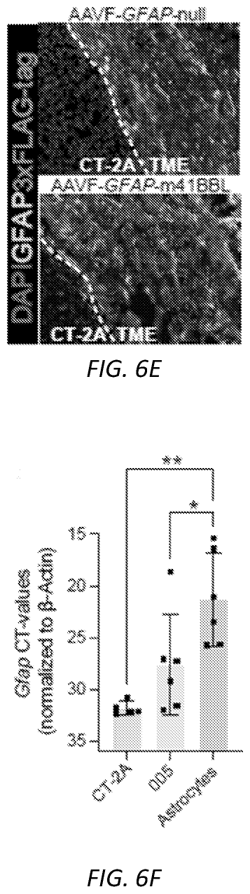

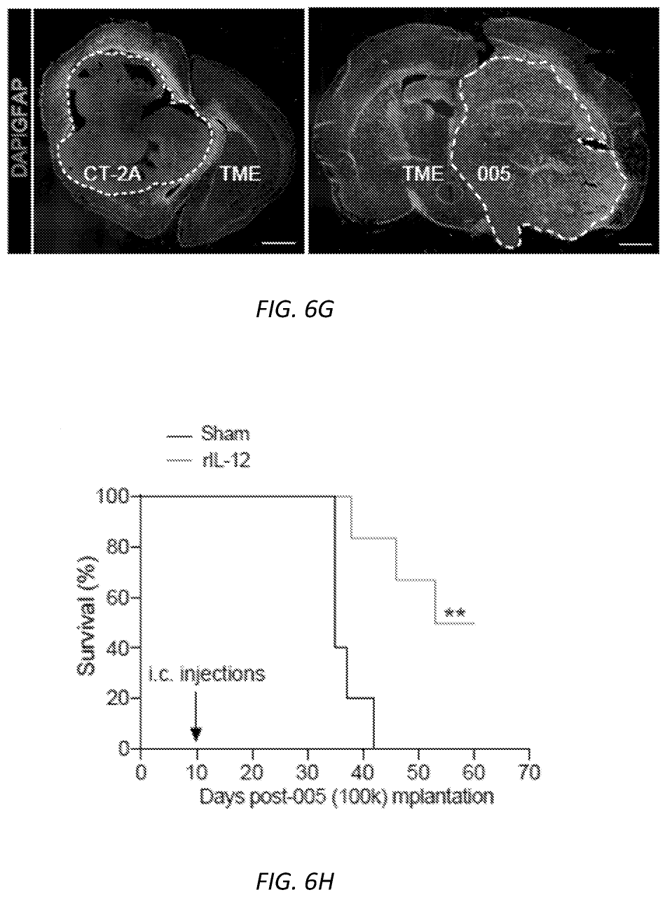

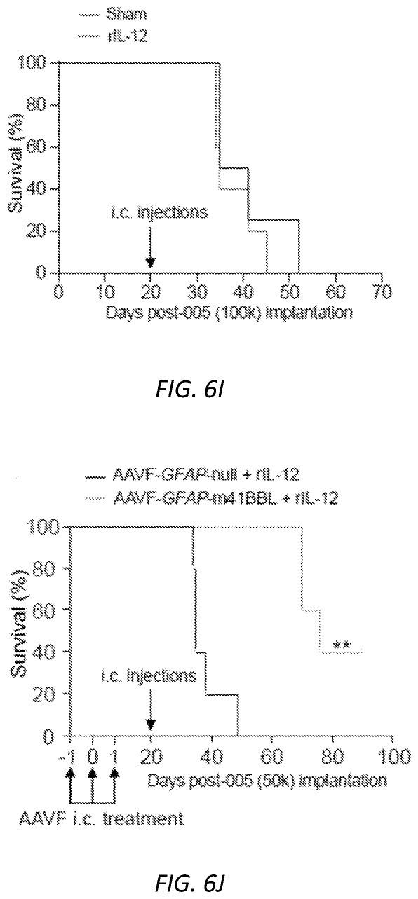

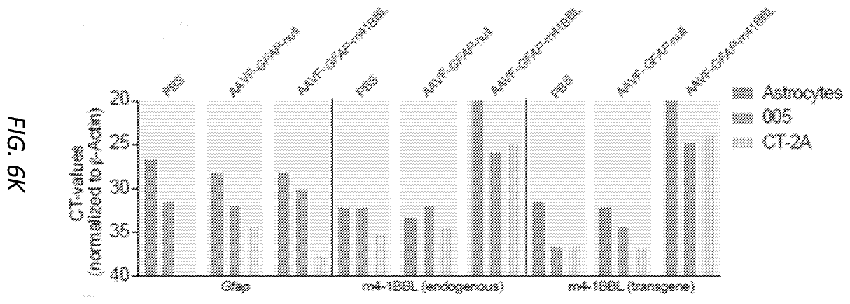

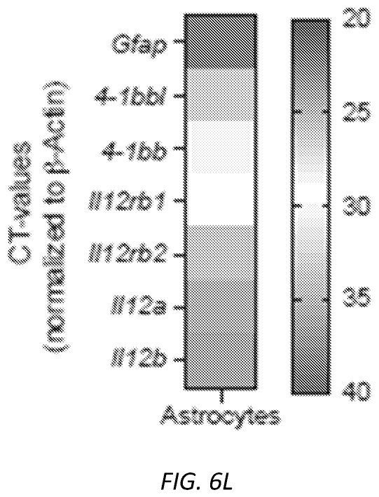

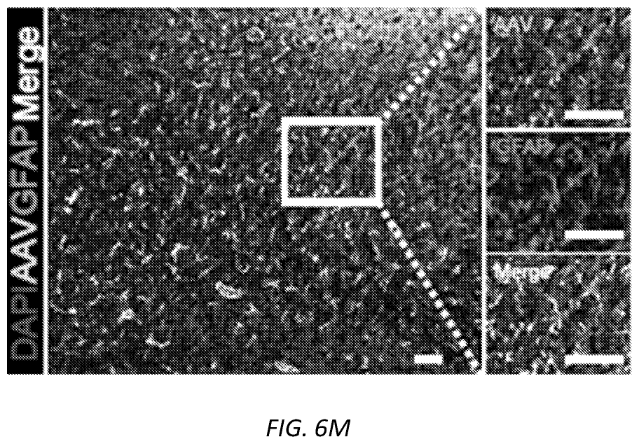

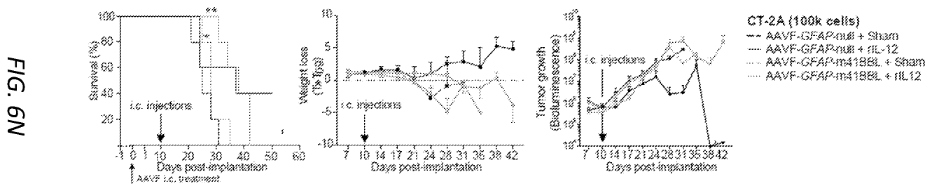

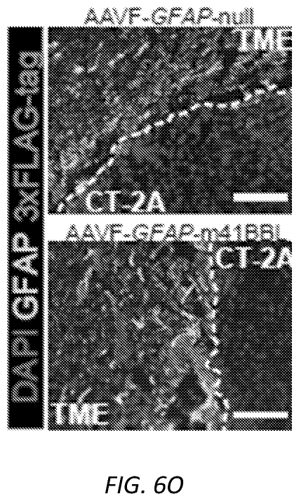

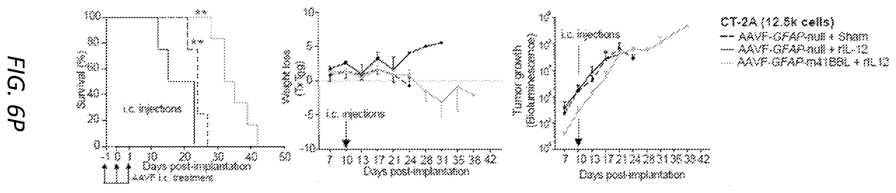

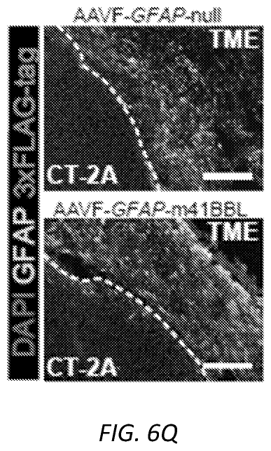

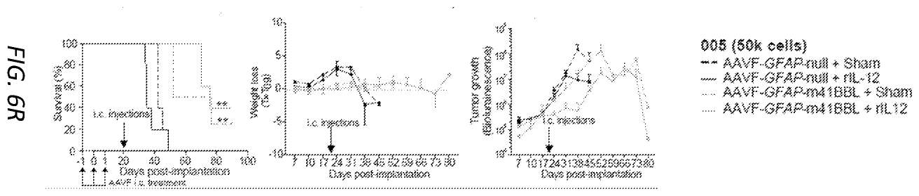

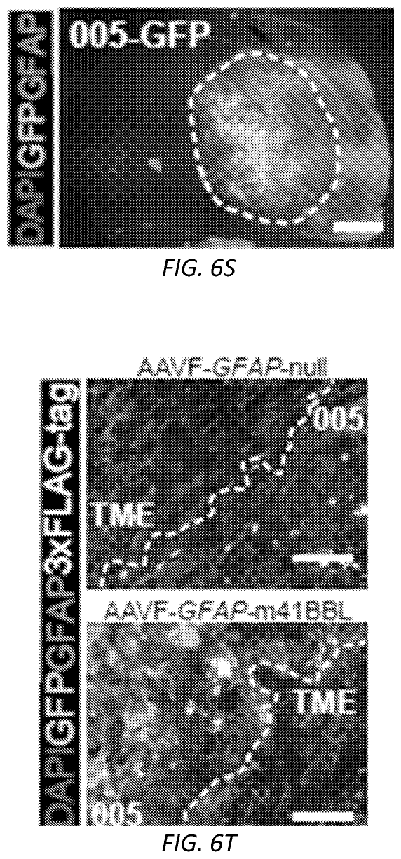

To transform our findings into a translatable therapy for GB patients, we developed an AAV vector expressing m4-1BBL. Immuno-gene therapy offers the advantage of targeting cells in the vicinity of the tumor without the need to transduce all tumor cells to achieve an anti-tumoral effect. This addresses a common issue observed with anti-oncogenic transgenes delivered by non-integrating vectors, which tend to become diluted during tumor cell proliferation and do not reach all tumor cells, allowing non-transduced tumor subsets to repopulate the brain (Hadaczek et al., 2005; Volak et al., 2018). In light of this, we chose to utilize an astrocyte specific promoter which is active in peritumor region, close to tumor-associated CTLs that can recognize tumor antigens but are hypofunctional due to immune suppression of the tumor (Jiang et al., 2020). We opted for a GFAP promoter that is highly active in reactive in astrocytes surrounding the tumor (Campbell et al., 2020). AAV vector plasmids were designed accordingly, encoding m4-1BBL-3×FlagTag driven by a GFAP promoter and a control AAV vector plasmid that had the same components, including the GFAP promoter but lacking 3×Flag-tag and m4-1BBL (FIG. 6A). These plasmids were packaged into an AAV-F capsid to generate AAVF-GFAP-m4-1BBL and AAVF-GFAP-null vectors. The AAV-F serotype was chosen for its robust transduction of astrocytes in the peritumoral region of GB (Volak et al., 2018). To validate m4-1BBL expression in i.e. AAVF-GFAP-m4-1BBL treated mice, mouse brains with tumor were analyzed through anti-3×FLAG-tag western blot analysis to confirm the presence of full-length recombinant m4-1BBL, not present in AAVF-GFAP-null treated GB mice (FIG. 6B). We next verified that m4-1BBL can be expressed in primary isolated mouse astrocytes post-transduction with AAVF-GFAP-m4-1BBL (FIG. 6K). We observed transgene expression preference of AAVF-GFAP-m4-1BBL in astrocytes vs CT-2A cells based on levels of GFAP, m4-1BBL transgene (using primers that target the 3×FLAG-tag region of the construct excluding endogenous 4-1BBL detection), and m4-1BBL (using primers that target both endogenous 4-1BBL and the m4-1BBL transgene) and compared these values to AAVF-GFAP-null and PBS treatment controls (FIG. 6K). Of note, Gfap expressing astrocytes may also have additional immunomodulatory functions of importance for this strategy, as they express transcripts for the following proteins: IL12rb1, IL12rb2, 4-1BB, and endogenous m4-1BBL, but not IL-12a and IL-12b (FIG. 6L). To confirm that astrocytes in the tumor border were targeted with our capsid and promoter, AAVF-GFAP-GFP was administered i.e. and showed successful targeting of GFAP^{POS} cells (FIG. 6M).

Similar to the VDX trials where a gene therapy vector was injected after tumor resection, we tested i.e. injection of AAVF-GFAP-m4-1BBL at the time of tumor implantation (100,000 Fluc-CT-2A cells). A single dose of AAV was followed by an i.e. injection with sham/rIL-12 10 days later (FIG. 6N). Mice treated with AAVF-GFAP-m4-1BBL, and rIL-12 showed significant improved survival compared to sham treatment, however there was no added advantage of AAVF-GFAP-m4-1BBL treatment over AAVF-GFAP-null both with sham or rIL-12 (FIG. 6N). Similar conclusions could be drawn on body weight and bioluminescence signal of GB-bearing mice (FIG. 6N), even though not a lot of transgene expression could be detected at the tumor border (white dotted line) based on anti-3×FLAG-tag and co-localized with anti-GFAP staining (FIG. 6O), we therefore repeatedly injected the AAVF-GFAP-m4-1BBL in further experiments. Of note, anti-3×FLAG-tag staining was not detected in AAVF-GFAP-null treated brains.

Hypothesizing that in GB patients, only a relatively small number of tumor cells remain present after tumor resection, we slightly changed our initial approach by engrafting fewer tumor cells (12,500 Fluc-CT-2A cells). Moreover, we considered that multiple doses of AAV, within a timeframe that no AAV-immunogenicity could be triggered (Verdera et al., 2020), might boost transgene expression and we injected three boli of AAVF-GFAP-m4-1BBL over 3 days (FIG. 6C). To model the use of intra-operative AAVF therapy in the tumor cavity, the first bolus was given one day prior to tumor cell implantation, the second one concomitant with the CT-2A implantation, and the third one, one day after tumor implantation. Ten days after tumor implantation, mice were treated with sham or rIL-12 and the survival of tumor-bearing mice was monitored (FIGS. 6D and 6P). In this case AAVF-GFAP-m4-1BBL with rIL-12 treatment showed significantly improved survival with a median of 33.5 days upon combined therapy compared to a 19-day median survival with rIL-12 and AAVF-GFAP-null. Again, we could demonstrate increased transgene expression by the presence of 3×FLAG-tag in GFAP-expressing cells, 3×FLAG-tag was more abundantly present compared to a single AAV injection, at the tumor border (white dotted line) in AAVF-GFAP-m4-1BBL treated mice compared to AAVF-GFAP-null treatment (FIGS. 6E and 6Q). Similar benefit as survival outcomes could be drawn from body weight and Fluc bioluminescence measurements (FIG. 6P).

To evaluate whether our immuno-gene therapy is tumor type dependent, we injected 005-Fluc glioma cells in syngeneic mouse model (Marumoto et al., 2009). 005 cells are an invasive glioma cell line that has an astrocyte lineage, and thus are expected to have high GFAP levels but still lower than compared to astrocytes (FIG. 6F). 005-Fluc cells not only express Gfap mRNA, but also the tumor border has a high number of reactive astrocytes with increased levels of GFAP (Parmigiani et al., 2021) (FIG. 6G). Compared to the densely packed GFAP^{POS} cells around the CT-2A tumor, GFAP^{POS} cells were more widespread in the 005-Fluc-bearing brain. Interestingly, the 005 model showed more GFAP^{POS} cells within the tumor, confirming their astrocytic background. GFAP^{POS} cells were co-localized with GFP in tumor burden of the 005-GFP implanted cells (FIGS. 6G and 6S).

Intratumoral rIL-12 with 10-day treatment was very effective in 005-Fluc glioma-bearing mice with a significant overall survival of 56.5 days compared to 35 days in the sham group (FIG. 6H). This could be reasoned because, compared to the CT-2A-glioma model, the 005-glioma model has a characteristic slow growing and more diffuse tumor phenotype that is more reflective of human GB. We therefore delayed the rIL-12 treatment to day 20 (half time of the survival), which had a reduced rIL-12 therapy effect (FIG. 6I). Next, we tested if this reduced therapeutic rIL-12 effect in the 005-glioma model could be boosted with our three injections of AAVF-GFAP-m4-1BBL co-therapy approach. Mice implanted with glioma cells (50,000 005-Fluc), showed improved outcome compared to the CT-2A model with a significant median survival of >60 days compared to 35 days in the control group (FIG. 6J). Similar conclusions could be drawn from body weight and Fluc bioluminescence measurements (FIG. 6R). We should note that rIL-12 did not augment our AAVF-GFAP-m41BBL treatment, indicating that AAVF-GFAP-m41BBL is sufficient for inducing anti-tumor immunity by itself even when IL-12 is administered too late to have an effect (i.e., day 20 instead of day 10 treatment). The number of detected 3×FLAG-tag—expressing cells (supporting m4-1BBL expression) was increased in GFAP-positive astrocytes at the 005-Fluc tumor border in the AAVF-GFAP-m41BBL compared to the null vector (FIG. 6T). We also observed 3×FLAG-tag staining in GFP^{POS} cells. GFP is a reporter of 005 cells, indicating that potentially both astrocytes and the tumor were driving m4-1BBL expression post-immuno-gene therapy. Taken together, our results indicate that our therapy approach combining rIL-12 with AAV was able to express m4-1BBL in the GFAP^{POS} cells at the GB site. Overall, we have demonstrated the therapeutic merit of stimulating CTLs with cytokine-based immunotherapy against GB.

### OTHER EMBODIMENTS

It is to be understood that while the subject matter has been described in conjunction with the detailed description thereof, the foregoing description is intended to illustrate and not limit the scope of the appended claims. Other aspects, advantages, and modifications are within the scope of the following claims.

What is claimed is:

1. A method of treating glioblastoma, the method comprising administering to a subject having glioblastoma a therapeutically effective amount of 4-1BBL in combination with interleukin 12 (IL-12), optionally recombinant IL-12 (rIL-12).

2. The method of claim 1, wherein the 4-1BBL is administered alone without IL-12.

3. The method of claim 1, wherein the rIL-12 comprises a fusion protein of IL-12 conjugated to Fc.

4. The method of claim 1, wherein the 4-1BBL comprises human 4-1BBL (h4-1BBL).

5. The method of claim 1, wherein the 4-1BBL is administered one or more times.

6. The method of any of claims 1, and 2 to 4, wherein the 4-1BBL is administered before, concurrently with, or after the IL-12.

7. The method of any of claims 1 to 5, wherein the method of treating glioblastoma comprises administering to the subject a vector encoding 4-1BBL.

8. The method of claim 7, wherein the vector is a lentiviral vector.

9. The method of claim 7, wherein the vector is an adeno-associated virus (AAV) vector.

10. The method of claim 9, wherein the AAV vector is an AAV-F capsid or an AAV-9 capsid.

11. The method of any of claims 7 to 10, wherein the vector comprises a GFAP promoter.

12. The method of any of claims 6 to 10, wherein administering the vector comprises intracranial or intratumoral administration.

13. The method of any of claims 1 to 12, wherein the glioblastoma comprises primary glioblastoma or recurrent glioblastoma.

14. A recombinant 4-1BBL and IL-12 composition for use in a method of treating glioblastoma.

15. The use of claim 14, wherein the 4-1BBL is administered before, concurrently with, or after the IL-12.

16. The use of claim 14, wherein the 4-1BBL is administered one or more times.

17. The use of any of claims 14 to 16, wherein the method of treating glioblastoma comprises administering to the subject a vector encoding 4-1BBL.

18. The use of claim 17, wherein the vector is a viral vector.

19. The use of any of claims 17 to 18, wherein the viral vector is a lentiviral vector.

20. The use of any of claims 17 to 18, wherein the viral vector is an adeno-associated virus (AAV) vector.

21. The use of claim 20, wherein the AAV vector is an AAV-F capsid.

22. The use of any of claims 18 to 21, wherein the viral vector comprises a GFAP promoter.

23. The use of any of claims 17 to 22, wherein administering the vector comprises intracranial or intratumoral administration.

24. The use of any of claims 14 to 23, wherein the glioblastoma comprises primary glioblastoma or recurrent glioblastoma.

25. A composition comprising an AAV-F vector comprising a GFAP promoter operably linked to a sequence encoding 4-1BBL.

26. The composition of claim 25, wherein the 4-1BBL is human 4-1BBL.

27. The composition of claim 25, wherein the 4-1BBL is mouse 4-1BBL.
